(19) United States
(12) Patent Application Publication
(10) Pub. No.: US 2010/0047185 A1
(43) Pub. Date: Feb. 25, 2010

(54) ACTVIE SUBSTANCE COMBINATION

(75) Inventors: Olga Gorchs Capa, Torello (ES); Oscar Jimenez Alonso, Igualada (ES); Carlos Ramon Trullas Cabanas, Viladecavalls (ES); Jordi Corbera Arjona, Terrassa (ES); David Panyella Costa, Badalona (ES)

Correspondence Address: ROBERTS MLOTKOWSKI SAFRAN & COLE, P.C.;Intellectual Property Department, P.O. Box 10064, MCLEAN, VA, 22102-8064, US

(73) Assignee: ISDIN S.A., Barcelona (ES)

(21) Appl. No.: 12/522,890

(22) PCT Filed: Jan. 11, 2008
(86) PCT No.: PCT/EP2008/000172

§ 371 Date: Aug. 28, 2009

(30) Foreign Application Priority Data
Jan. 12, 2007 (EP) 07000631.7

(51) Int. Cl. A61K 9/12; A61K 8/49; A61Q 17/04

(52) U.S. Cl. 424/45; 424/59

(57) ABSTRACT

The present invention refers to a combination of at least 2 UV-absorbing active substances (A) and (B), wherein the UV-absorbing active substance (A) is at least one heptaazaphenalene compound of the general formula (I), which is defined in the description and the UV-ab-sorbing active substance (B) is at least one compound which has its ab-sorption maximum λ_{maX(B)} in the range of UV-A or UV-B radiation.

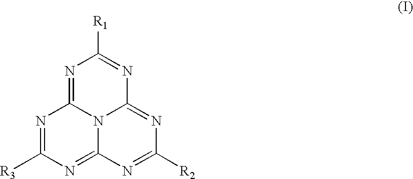

The present invention refers to a combination of at least 2 UV-absorbing active substances (A) and (B),

wherein
the UV-absorbing active substance (A) is at least one heptaazaphenalene compound of the general formula (I),

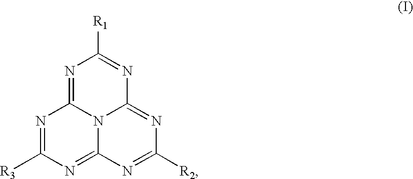

wherein the radicals R₁, R₂ and R₃ are defined as hereinafter, which compound has its absorption maximum λₘₐₓ in the range of UV-A or UV-B radiation,
and the UV-absorbing active substance (B)
is at least one compound, which has its absorption maximum λ_{max(B)} in the range of UV-A or UV-B radiation, preferably in such UV radiation range in which the λ_{max(A)} of UV-absorbing active substance (A) does not lie,
and wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1,
to a pharmaceutical or a cosmetical composition comprising said combination,
to the use of the combination of said active substances for the production of a medicament for the prophylaxis and/or treatment of diseases caused by and/or associated with exposure to ultraviolet radiation and the to use of said combination for protecting the skin of mammals.

Ultraviolet (UV) radiation is part of the electromagnetic spectrum emitted by the sun. Whereas UV-C radiation (wavelength [λ] range of 100-290 nm; λ₁₀₀₋₂₉₀) are absorbed by the atmospheric ozone, most radiation in the UV-A range of 320-400 nm (λ₃₂₀₋₄₀₀) and about 10% of the UV-B radiation range of 290-320 nm (λ₂₉₀₋₃₂₀) reach the earth's surface. UV-A as well as UV-B radiation is of major importance for human health.

Small amounts of UV radiation are essential for the production of vitamin D in mammals, especially in human beings, yet overexposure may result in acute and chronic health effects of the skin, eye and/or the immune system. To prevent damage of the skin caused by UV-A and UV-B radiation, human beings have developed various natural protection systems either to absorb or to deflect said radiation, like the production of melanin, a fatty layer on the skin, and/or the growth of hair.

In addition, UV-absorbing substances have been developed in order to prevent and/or reduce the effects of solar radiation. Such UV-absorbing substances are compounds that are applied to the skin, lips, nails and/or hair of mammals, especially human beings, in form of additives in cosmetic, dermatological and pharmaceutical formulations.

Despite the great diversity of commercially available sunscreens, there still exists a need for sunscreens, which provide at least a high effectiveness either in the UV-A or the UV-B radiation range, preferably in both ranges, since the sunlight radiation intensity, and in consequence the UV radiation, has been increasing continuously. As a consequence there exists a need for a sufficient protection against such increased UV radiation in order to reduce the risk of suffering of diseases such as skin cancer, polymorphous light eruption, actinic keratosis, solar urticaria, xeroderma pigmentosum, photoageing and/or chronic actinic dermatitis caused by UV-A and/or UV-B radiation.

Therefore, it was an object of the present invention to provide a new and effective sun screen system for absorbing preferably at least at a higher level either in the UV-A or UV-B radiation range, preferably in both radiation ranges, so that a remarkable protection against these UV radiation is achieved, especially in order to provide enough care and prophylaxis or treatment against diseases caused by or associated with the exposure of skin to UV radiation.

This object is solved by the provision of the inventive combination of at least 2 UV-absorbing active substances (A) and (B),

wherein
the UV-absorbing active substance (A) is at least one heptaazaphenalene compound of the general formula (I),

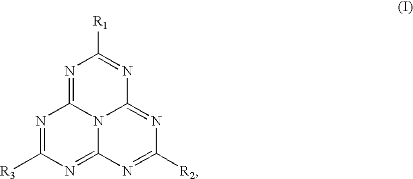

wherein the radicals R₁, R₂ and R₃ are defined hereinafter, and which compound has its absorption maximum λ_{max(A)} either in the range of UV-A or UV-B radiation,
and
the UV-absorbing active substance (B) is at least one compound, which has its absorption maximum λ_{max(B)} either in the range of UV-A or UV-B radiation, preferably the λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.
with the exception of the combination of 37.5% by weight, based on the weight of the combination, of 2,5,8-tris-(4-(butoxycarbonyl)phenylamino)-1,3,4,6,7,9,9b-heptaazaphenalene

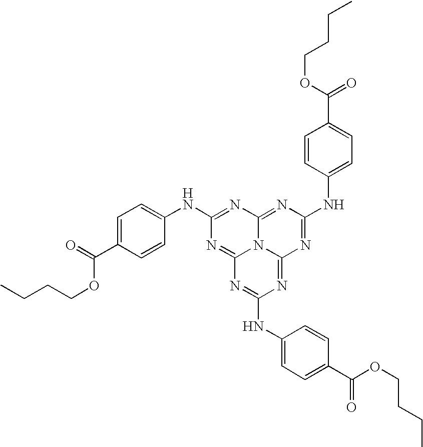

and 62.5% by weight, based on the weight of the combination, of ethylhexyl methoxycinnamate;
with the exception of the combination of 60.5 to 67.6% by weight, preferably 60 to 70% by weight, based on the weight of the combination, of 2-(4-(carboxy)phenylamino)-5,8-bis-(4-methylphenyl)-1,3,4,6,7,9,9b-heptaazaphenalene

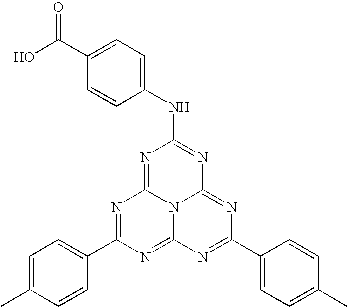

and 32.4 to 39.5% by weight, preferably 30 to 40% by weight, based on the weight of the combination, of titanium dioxide,
and
with the exception of the combination of 60.5 to 67.6% by weight, preferably 60 to 70% by weight, based on the weight of the combination, of 2,5,8-tris-(4-(2-ethylhexyloxycarbonyl)phenylamino)-1,3,4,6,7,9,9b-heptaazaphenalene

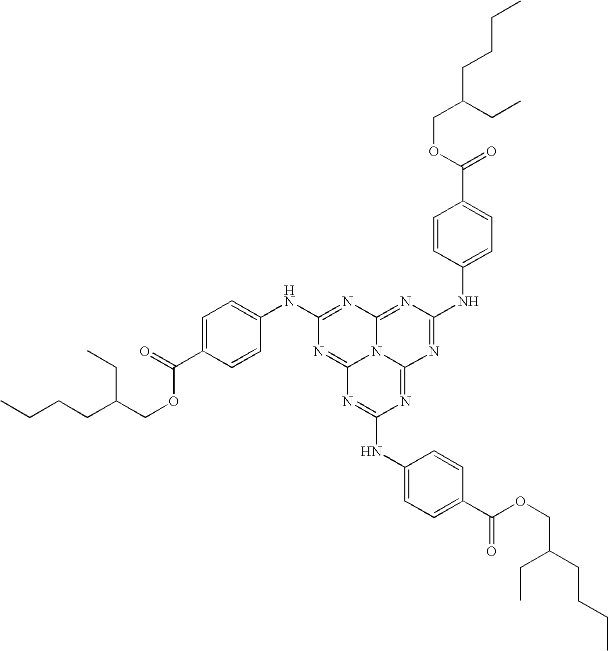

and 32.4 to 39.5% by weight, preferably 30 to 40% by weight, based on the weight of the combination, of titanium dioxide.

By means of providing such absorbing inventive combination an effective sun protection system is obtained against either UV-A or UV-B radiation, or, preferably a complementary UV-absorbing system for absorbing UV-A and UV-B radiation to a considerable extent is obtained. Additionally, the inventive combination allows for an effective absorption of UV radiation either in the UV-A or the UV-B or even in both UV-A and UV-B radiation ranges at a sufficiently high level, i.e. it absorbs UV radiation from either 320 to 400 nm, 290 to 320 nm or 290 to 400 nm at least in such amount that an adequate protection for the skin is provided.

According to the present invention the UV-absorbing active substance (A) is at least one heptaazaphenalene compound of the general formula (I),

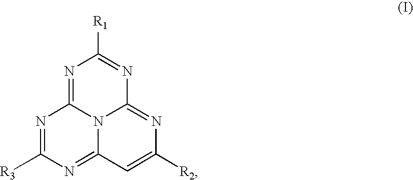

wherein

R₁, R₂ and R₃, independent of each other, each represents an unsubstituted or at least mono-substituted mono- or polycyclic aryl radical; an unsubstituted or at least mono-substituted, saturated, unsaturated or aromatic heterocyclic radical having from to 10 atoms that can contain 1, 2 or 3 heteroatoms independently selected from O, N and S; an —OR₄ radical; or a —NR₅R₆ radical;

R₄ represents a hydrogen atom; an unsubstituted or at least mono-substituted, saturated or unsaturated, linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted, saturated, unsaturated or aromatic heterocyclic radical having from 5 to 14 atoms that can contain 1, 2 or 3 heteroatoms independently selected from O, N and S;

R₅ and R₆, independent of each other, each represents a hydrogen atom; an unsubstituted or at least mono-substituted, saturated or unsaturated, linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted, saturated, unsaturated or aromatic heterocyclic radical having from 5 to 10 atoms that can contain 1, 2 or 3 heteroatoms independently selected from O, N and S;

or

R₅ and R₆ form together with the bridging nitrogen atom an unsubstituted or at least mono-substituted saturated, unsaturated or aromatic mono- or polycyclic ring system having from 4 to 10 atoms that contains optionally 1 or 2 heteroatoms independently selected from N, O and S;

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
preferably with the proviso that if R₁, R₂ and R₃ are identical, R₁, R₂ and R₃ shall at the same time not represent an unsubstituted phenyl radical;
preferably with the proviso that if R₁, R₂ and R₃ each represents an —OR₄ radical at the same time, R₄ shall not represent a hydrogen atom or a radical selected from the group consisting of n-butyl, ethyl, phenyl, benzyl, 2,6-dimethylphenyl, 3,5-dimethylphenyl, 2,2,3,3,4,4,4-heptafluorobutyl, 2,2,3,3,3-pentafluoropropyl, 2,2,2-trifluoroethyl and hydroxymethyl;
preferably with the proviso that if R₁, R₂ and R₃ each represents an —OR₄ radical at the same time, and if R₄ represents an ethyl radical for two of the radicals R₁, R₂ and R₃, then R₄ shall not represent a hydrogen atom for the third radical of the radicals R₁, R₂ and R₃;
preferably with the proviso that if R₁, R₂ and R₃ each represents a —NR₅R₆ radical at the same time, R₅ and R₆, being identical, shall not represent a hydrogen atom or a radical selected from the group consisting of ethyl, n-butyl, benzyl, n-heptyl, unsubstituted phenyl, cyclohexyl, 2-pyridyl and hydroxymethyl;
preferably with the proviso that if R₁, R₂ and R₃ each represents a —NR₅R₆ radical at the same time, and if either R₅ or R₆ represents a hydrogen atom, the other of the radicals R₅ and R₆ shall not represent a radical selected from the group consisting of n-butyl, unsubstituted phenyl, hydroxymethyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl and 5-benzoylamino-9,10-dihydro-9,10-dioxoanthracene-1-yl;
preferably with the proviso that if R₁, R₂ and R₃ each represents a —NR₅R₆ radical at the same time, and if either R₅ or R₆ represents a phenyl radical, the other of the radicals R⁵ and R⁶ shall not represent a methyl radical;
preferably with the proviso that if R₁, R₂ and R₃ each represents a —NR₅R₆ radical at the same time, and if R₅ and R₆ represent at the same time a n-heptyl radical and are identical for two of the radicals R₁, R₂ and R₃, and if the third of the radicals R₁, R₂ and R₃ represents a —NR₅R₆ radical, R₅ and R₆ shall at the same time not represent a phenyl radical for this third radical;
and
preferably with the proviso that if R₁, R₂ and R₃ each represents a —NR₅R₆ radical at the same time, and if R₅ and R₆ represent at the same time a phenyl radical and are identical for two of the radicals R₁, R₂ and R₃, and if the third radical of R₁, R₂ and R₃ represents a —NR₅R₆ radical, then R₅ and R₆ shall at the same time not represent a n-heptyl radical for this third radical,
which active substance (A) has its absorption maximum λ_{max(A)} either in the range of UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1; preferably from 1:10 to 20:1.

In a preferred embodiment of the present invention, the UV-absorbing active substance (A) is at least one heptaazaphenalene compound of general formulae (IA) to (IL)

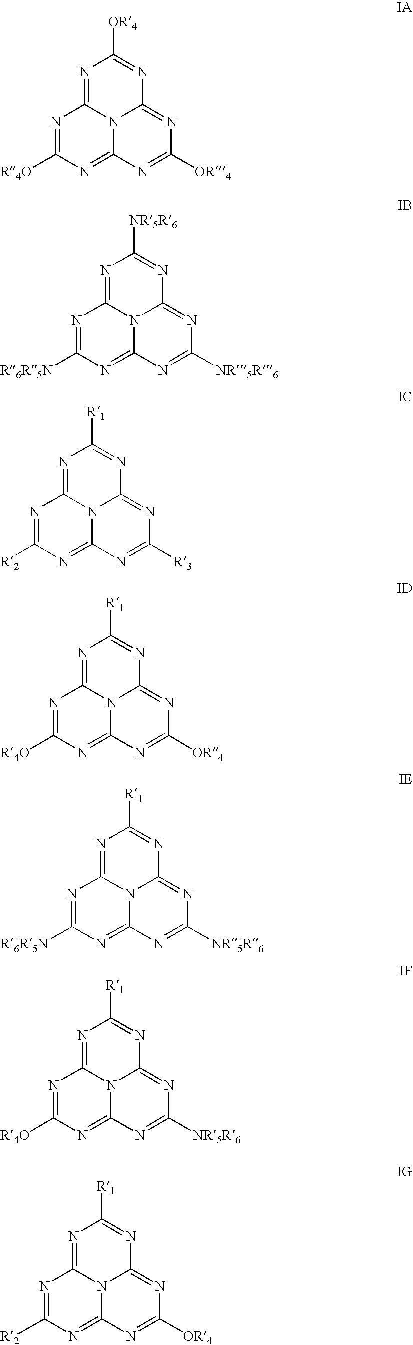

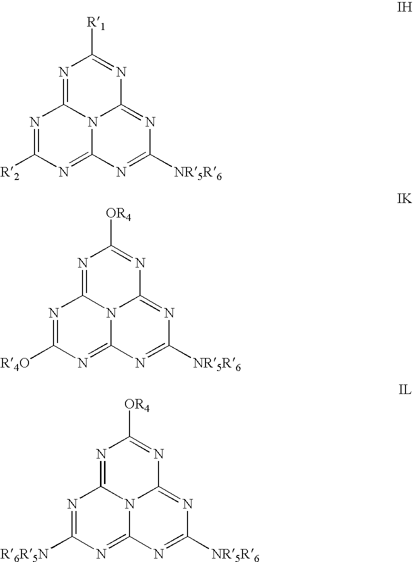

wherein

R′₁, R′₂ and R′₃, independent of each other, each represents an unsubstituted or at least mono-substituted, mono- or polycyclic aryl radical; an unsubstituted or at least mono-substituted, saturated, unsaturated or aromatic heterocyclic radical having from 5 to 10 atoms that can contain 1, 2 or 3 heteroatoms independently selected from O, N and S;

R₄, R′₄, R″₄ and R′″₄, independent of each other, each represents a hydrogen atom, an unsubstituted or at least mono-substituted, saturated or unsaturated linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted saturated, unsaturated or aromatic heterocyclic radical having from 5 to 14 atoms that can contain 1, 2 or 3 heteroatoms independently selected from O, N and S;

R′₅, R″₅, R′″₅, R′₆, R″₆ and R′″₆, independent of each other, each represents a hydrogen atom; an unsubstituted or at least mono-substituted, saturated or unsaturated, linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted saturated, unsaturated or aromatic heterocyclic radical having from 5 to 10 atoms that can contain 1, 2 or 3 heteroatoms independently selected from O, N and S;

or

R′₅, R″₅, R′″₅, R′₆, R″₆ and R′″₆, form together with the bridging nitrogen atom an unsubstituted or at least mono-substituted, saturated, unsaturated or aromatic mono- or polycyclic ring system which has 5 to 10 atoms as ring members and which contains optionally 1, 2 or 3 heteroatoms independently selected from O, N and S;

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (IA)

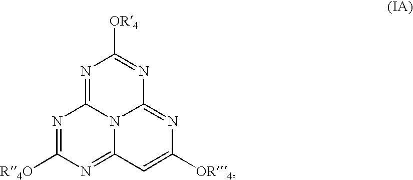

wherein

R′₄, R″₄ and R′″₄, independent of each other, each represents an unsubstituted or at least mono-substituted, C₃₋₁₂ cycloalkyl radical; an unsubstituted or at least mono-substituted mono- or polycyclic aryl radical; an unsubstituted or at least mono-substituted saturated, unsaturated or aromatic heterocyclic radical having from 5 to 14 atoms that can contain 1, 2 or 3 heteroatoms independently selected from O, N and S;

preferably with the aforementioned provisos; and
preferably with the proviso that if R′₄, R″₄ and R′″₄ are at the same time identical, they shall not represent phenyl, benzyl, 2,6-dimethylphenyl or 3,5-dimethylphenyl;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound as defined above,

wherein

R₄, R′₄, R″₄ and R′″₄, identical or different, each represents an unsubstituted or at least mono-substituted aryl group, wherein said substituent(s) are independently from each other selected from the group consisting of an unsubstituted or at least mono-substituted C₃₋₁₂ cycloalkyl radical; an unsubstituted or at least mono-substituted C₂₋₆ alkenyl radical; an unsubstituted or at least mono-substituted aryl radical; an unsubstituted or at least mono-substituted, saturated, unsaturated or aromatic heterocyclic radical having from 5 to 10 atoms that can contain 1, 2 or 3 heteroatoms independently selected from O, N and S; a —C(═O)—OR₇ radical; a —C(═O)NR₈R₉ radical; a —COR₁₀ radical; an —OH radical; F, Cl, Br or I; an unsubstituted or at least mono-substituted, linear or branched —O—C₁₋₈ radical; an unsubstituted or at least mono-substituted, linear or branched C₁₋₆ alkyl radical, wherein the substituent(s) for the aforementioned —O—C₁₋₈ radical and/or the aforementioned C₁₋₆ alkyl radical represent, independently from each other, an —OH radical; a —SO₃M radical; a —N(R₁₁)₂ radical; a —N(R₁₁)₃⁺ radical or a moiety of general formula (II):

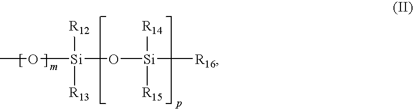

wherein
m=0 or 1;
p=0, 1, 2, 3 or 4

R₁₂, R₁₃, R₁₄, R₁₅ and R₁₆, identical or different, each represents an unsubstituted or at least mono-substituted, linear or branched C₁₋₆ alkyl radical; an unsubstituted or at least mono-substituted, linear or branched —O—C₁₋₆ radical; an unsubstituted or at least mono-substituted aryl radical or an —OSi(R₁₇)₃ radical;

R₁₇ represents an unsubstituted or at least mono-substituted, linear or branched C₁₋₆ alkyl radical, an unsubstituted or at least mono-substituted, linear or branched —O—C₁₋₈ radical or an unsubstituted or at least mono-substituted aryl radical;

M represents H, Na⁺ or K⁺;

R₇, R₈ and R₉, independent from each other, each represents a hydrogen atom; an unsubstituted or at least mono-substituted aryl radical; an unsubstituted or at least mono-substituted, linear or branched C₁₋₁₈ alkyl radical; an unsubstituted or at least mono-substituted, C₃₋₁₂ cycloalkyl radical; an unsubstituted or at least mono-substituted saturated, unsaturated or aromatic heterocyclic radical having from 5 to 10 atoms that can contain 1, 2 or 3 heteroatoms independently selected from O, N and S;

or

R₈ and R₉ form together with the bridging nitrogen atom an unsubstituted or at least mono-substituted, saturated, unsaturated or aromatic mono- or polycyclic ring system having from 5 to 10 atoms as ring members that contains optionally 1 or 2 heteroatoms independently selected from O, N and S;

R₁₀ represents an unsubstituted or at least mono-substituted, linear or branched alkyl radical, or an unsubstituted or at least mono-substituted aryl radical,

or

R₁₀ is fused to form a mono- or polycyclic saturated, unsaturated or aromatic ring system having from 5 to 10 atoms as ring members that contains optionally 1 or 2 heteroatoms independently selected from O, N and S;

R₁₁ represents an unsubstituted or at least mono-substituted, linear or branched alkyl radical;

preferably with the aforementioned provisos; and
preferably with the proviso that when R₁, R₂ and R₃ represent at the same time the same substituent, R₄ shall not represent a phenyl, benzyl, 2,6-dimethylphenyl or 3,5-dimethylphenyl radical;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (IB)

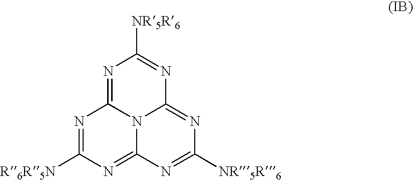

wherein
the radicals within each radical pair R′₅ R′₆, respectively, R″₅ R″₆, respectively, R′″₅R′″₆, different from each other, each represents a hydrogen atom; an unsubstituted or at least mono-substituted, linear or branched C₁₋₁₈ alkyl radical; an unsubstituted or at least mono-substituted C₃₋₁₂ cycloalkyl radical; an unsubstituted or at least mono-substituted mono- or polycyclic aryl radical; an unsubstituted or at least mono-substituted, saturated, unsaturated or aromatic heterocyclic radical having from 5 to 10 atoms that can contain 1, 2 or 3 heteroatoms independently selected from O, N and S;
or
the radical pairs R′₅ R′₆, respectively, R″₅ R″₆, respectively, R′″₅R′″₆, form together with the bridging nitrogen atom an unsubstituted or at least mono-substituted, saturated, unsaturated or aromatic mono- or polycyclic ring system having from 4 to 10 atoms as ring members that contains optionally 1 or 2 heteroatoms selected from N, O and S;
preferably with the aforementioned provisos;
preferably with the proviso that if one of the radicals within each radical pair R′₅ R′₆, respectively R″₅ R″₆, respectively R′″₅R′″₆ is hydrogen, the other radical is different from n-butyl, phenyl, hydroxymethyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl;
and
preferably with the proviso that if one of the radicals within each radical pair R′₅ R′₆, respectively R″₅ R″₆, respectively R′″₅R′″₆ is phenyl, the other radical shall not represent a methyl radical;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound as described above,

wherein
one radical of the pair R₅R₆ or optionally one radical of the pair R′₅ R′₆, respectively R″₅ R″₆, respectively R′″₅R′″₆ represents an unsubstituted or at least mono-substituted aryl radical, wherein said substituent(s) are, independently from each other, selected from the group consisting of an unsubstituted or at least mono-substituted, C₃₋₁₂ cycloalkyl radical; a linear or branched, unsubstituted or at least mono-substituted C₂₋₆ alkenyl radical; an unsubstituted or at least mono-substituted aryl radical; an unsubstituted or at least mono-substituted saturated, unsaturated or aromatic heterocyclic radical having from 5 to 10 atoms that can contain 1, 2 or 3 heteroatoms independently selected from O, N and S; a —C(═O)—OR₇ radical; a —C(═O)NR₈R₉ radical; a —COR₁₀ radical; —OH; F, Cl, Br, I; an unsubstituted or at least mono-substituted, linear or branched —O—C₁₋₈ radical; an unsubstituted or at least mono-substituted, linear or branched C₁₋₆ alkyl radical, wherein the substituent(s) for the aforementioned —O—C₁₋₈ radical and/or the aforementioned C₁₋₆ alkyl radical represent(s) independently from each other at least one —SO₃M radical, a —N(R₁₁)₂ radical, a —N(R₁₁)₃⁺ radical, or a moiety of general formula (II)

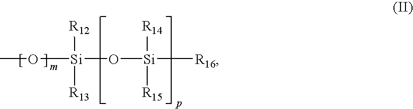

wherein
m=0 or 1;
p=0, 1, 2, 3 or 4;

R₁₂, R₁₃, R₁₄, R₁₅ and R₁₆, identical or different, each represents an unsubstituted or at least mono-substituted, linear or branched C₁₋₆ alkyl radical; an unsubstituted or at least mono-substituted, linear or branched —O—C₁₋₆ radical; an unsubstituted or at least mono-substituted aryl radical or an —OSi(R₁₇)₃ radical;

R₁₇ represents an unsubstituted or at least mono-substituted, linear or branched C₁₋₆ alkyl radical; an unsubstituted or at least mono-substituted, linear or branched —O—C₁₋₆ radical or an unsubstituted or at least mono-substituted aryl radical;

M represents H, Na⁺ or K⁺;

R₇, R₈ and R₉, identical or different, each represents a hydrogen atom; an unsubstituted or at least mono-substituted aryl radical; an unsubstituted or at least mono-substituted, linear or branched C₁₋₁₈ alkyl radical; an unsubstituted or at least mono-substituted, C₃₋₁₂ cycloalkyl radical or an unsubstituted or at least mono-substituted saturated, unsaturated or aromatic heterocyclic having from 5 to 10 atoms that can contain 1, 2 or 3 heteroatoms independently selected from O, N and S;

or

R₈ and R₉ form together with the bridging nitrogen atom an unsubstituted or at least mono-substituted, saturated, unsaturated or aromatic mono- or polycyclic ring system having from 5 to 10 atoms that contains optionally 1 or 2 heteroatoms independently selected from O, N and S;

R₁₀ represents an unsubstituted or at least mono-substituted, saturated or unsaturated, linear or branched C₁₋₆ alkyl radical or an unsubstituted or at least mono-substituted aryl radical;

or

R₁₀ is fused to form a mono- or polycyclic saturated, unsaturated or aromatic ring system having from 5 to 10 atoms that contains optionally 1 or 2 heteroatoms independently selected from O, N and S;

R₁₁ represents an unsubstituted or at least mono-substituted, linear or branched alkyl radical;

preferably with the aforementioned provisos;
preferably with the proviso that if one radical of the pair R₅R₆ or optionally one radical of the pair R′₅ R′₆, respectively R″₅ R″₆, respectively R′″₅R′″₆ represents an unsubstituted phenyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl radical, the other radical is different from hydrogen;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (IC)

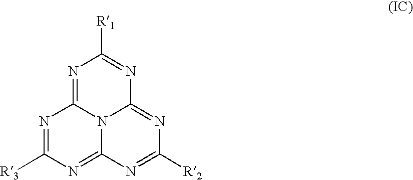

wherein

R′₁, R′₂ and R′₃, identical or different, each represents an unsubstituted or at least mono-substituted mono- or polycyclic aryl radical; an unsubstituted or at least mono-substituted saturated, unsaturated or aromatic heterocyclic radical having from 5 to 10 atoms and that can contain 1, 2 or 3 heteroatoms independently selected from O, N and S;

preferably with the aforementioned provisos;
preferably with the proviso that R′₁, R″₂ and R′″₃ shall not at the same time represent a phenyl, methylphenyl, dimethylphenyl, trimethylphenyl, and 2,3,5,6-tetramethylphenyl;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound as described above,

wherein

R₁, R₂ and R₃, or R′₁, R′₂ and R′₃, identical or different, each represents at least one radial selected from the group consisting of naphthyl, pyrrole, thiophene, indole, pyrazole, imidazole, triazole, benzothiophene, benzimidazole, benzopyrazole, oxazole, isoxazole, benzofurane, which is unsubstituted or at least mono-substituted;

or
represents a radical of general formula (III)

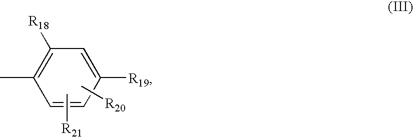

wherein

R₁₈ represents a hydrogen atom; —OH; an —OR₂₂ radical; an unsubstituted or at least mono-substituted, saturated or unsaturated, linear or branched C₁₋₁₈ alkyl radical; or an unsubstituted or at least mono-substituted, linear or branched —O—C₁₋₁₈ radical;

R₁₉ represents a hydrogen atom; —OH; an unsubstituted or at least mono-substituted aryl radical; an unsubstituted or at least mono-substituted saturated, unsaturated or aromatic heterocyclic radical having from 5 to 10 atoms that can contain 1, 2 or 3 heteroatoms independently selected from O, N and S; a —C(═O)—OR₇ radical; a —C(═O)NR₈R₉ radical; an —OR₂₂ radical; an unsubstituted or at least mono-substituted —COR₁₀ radical; an unsubstituted or at least mono-substituted, C₃₋₆ cycloalkyl radical; a saturated or unsaturated, linear or branched C₁₋₁₈ alkyl radical, which is unsubstituted or at least mono-substituted by at least one hydroxyl radical; a —SO₃M radical; a —N(R₁₁)₂ radical; a —N(R₁₁)₃⁺ radical, or by a group of general formula (II)

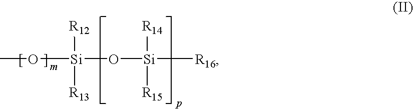

wherein
m=0 or 1;
p=0, 1, 2, 3 or 4;

R₁₂, R₁₃, R₁₄, R₁₅ and R₁₆, identical or different, each represents an unsubstituted or at least mono-substituted, linear or branched C₁₋₁₈ alkyl radical; an unsubstituted or at least mono-substituted, linear or branched —O—C₁₋₆ radical; an unsubstituted or at least mono-substituted aryl radical or an —OSi(R₁₇)₃ radical;

R₁₇ represents an unsubstituted or at least mono-substituted, linear or branched C₁₋₆ alkyl radical; an unsubstituted or at least mono-substituted, linear or branched —O—C₁₋₆ radical or an unsubstituted or an at least mono-substituted aryl radical;

M represents H, Na⁺ or K⁺;

R₇, R₈ and R₉, identical or different, each represents a hydrogen atom; an unsubstituted or at least mono-substituted aryl radical; an unsubstituted or at least mono-substituted, linear or branched C₁₋₁₈ alkyl radical; an unsubstituted or at least mono-substituted, C₃-C₁₂ cycloalkyl radical or an unsubstituted or at least mono-substituted saturated, unsaturated or aromatic heterocyclic radical having from 5 to 10 atoms that can contain 1, 2 or 3 heteroatoms independently selected from O, N and S;

or

R₈ and R₉ form together with the bridging nitrogen atom a saturated, unsaturated or aromatic mono- or polycyclic ring system having from 5 to 10 atoms as ring members that contains optionally 1 or 2 heteroatoms independently selected from O, N and S;

R₁₀ represents an unsubstituted or at least mono-substituted saturated or unsaturated, linear or branched C₁₋₆ alkyl radical, or an unsubstituted or at least mono-substituted aryl radical,

or

R₁₀ is fused to form an unsubstituted or at least mono-substituted, saturated, unsaturated or aromatic mono- or polycyclic ring system having from 5 to 10 atoms as ring members that contains optionally 1 or 2 heteroatoms independently selected from O, N and S;

R₁₁ represents an unsubstituted or at least mono-substituted, linear or branched alkyl radical;

R₂₂ represents an unsubstituted or at least mono-substituted aryl radical; an unsubstituted or at least mono-substituted saturated, unsaturated or aromatic heterocyclic radical having from 5 to 10 atoms that can contain 1, 2 or 3 heteroatoms independently selected from O, N and S; an unsubstituted or at least mono-substituted —COR₁₀ radical; an unsubstituted or at least mono-substituted C₃₋₁₂ cycloalkyl radical; a saturated or unsaturated linear or branched C₁₋₁₈ alkyl radical which is unsubstituted or at least mono-substituted by at least one —OH radical; a —SO₃M radical; a —N(R₁₁)₂ radical; a —N(R₁₁)₃⁺ radical or by a radical of general formula (II)

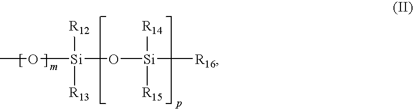

wherein
m=0 or 1;
p=0, 1, 2, 3 or 4
and wherein

R₇, R₈, R₉, R₁₀, R₁₁, R₁₂, R₁₃, R₁₄, R₁₅, R₁₆ and M have the above defined meaning;

R₂₀ and R₂₁, identical or different, each represents a hydrogen atom; an unsubstituted or at least mono-substituted, saturated or unsaturated, linear or branched C₁₋₆ alkyl radical; an unsubstituted or at least mono-substituted, linear or branched —O—C₁₋₆ radical or a —SO₃M radical, with M being H, Na⁺ or K⁺;

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound as described above,

wherein

R₄ represents a radical selected from the group consisting of 4-methoxyphenyl, naphthyl, cyclopentyl and cyclohexyl;

R₅, R′₅, R″₅, R′″₅, R₆, R′₆, R″₆ and R″₆ are different from each other and each represent a hydrogen atom, cyclopropyl, cyclohexyl, cyclobutyl, cycloheptyl, cyclopentyl, 4-(hydroxycarbonyl)phenyl, 4-(butoxycarbonyl)phenyl, 4-(2-ethylhexyloxycarbonyl)phenyl, 4-(2-butyloctyloxycarbonyl)phenyl, 4-(2-hexyldecyloxycarbonyl)phenyl, 4-(3,3,5-trimethylcyclohexyloxycarbonyl)phenyl, 4-(3,3,5-trimethylhexyloxycarbonyl)phenyl, 4-(octadecyloxycarbonyl)phenyl, 4-(hexadecyloxycarbonyl)phenyl, 4-(docecyloxycarbonyl)phenyl, 4-((2-ethylhexyl)carbamoyl)phenyl, 4-(L-menthyloxycarbonyl)phenyl, 4-styrylphenyl, 3-styrylphenyl, 3-((E)-3-(2-ethylhexyloxy)-3-oxoprop-1-enyl)phenyl, 4-((E)-3-(2-ethylhexyloxy)-3-oxoprop-1-enyl)phenyl, 4-methoxyphenyl, 3-methoxyphenyl, 3-nitrophenyl, phenyl, biphenyl-4-yl, 4-(imidazo[1,2-a]pyridin-2-yl)phenyl, 4-(4,5,6,7-tetrahydro-2,6,6-trimethyl-4-oxoindol-1-yl)phenyl, 4-(1H-benzo[d]imidazol-2-yl)phenyl, hydroxymethyl, pyridin, heptyl, butyl, ethyl, 2-ethylhexyl, 4-(1,3,3-trimethylbicyclo[2.2.1]heptan-2-yloxy)phenyl, 1H-indol-5-yl, 4-((3,7-dimethyloctyloxy)carbonyl)phenyl, 2-amino-4,5-dimethylphenyl or n-propyl;

R₇ represents a hydrogen atom, methyl, ethyl, n-propyl, isopropyl, n-butyl, tert-butyl, n-pentyl, n-hexyl, 2-ethylhexyl, L-menthyl, 3,3,5-trimethylcyclohexanyl, 3,3,5-trimethylhexanyl, dodecyl, 2-butyloctyl, 2-hexyldecyl, hexadecyl, octadecyl, 3,7-dimethyloctyl, 1,3,3-trimethylbicyclo[2.2.1]heptan-2-yl, unsubstituted or at least mono-substituted benzyl radical or an unsubstituted or at least mono-substituted phenyl radical;

R₈ and R₉, identical or different, each represents a hydrogen atom, methyl, ethyl, n-propyl, isopropyl, n-butyl, tert-butyl, n-pentyl, n-hexyl, 2-ethylhexyl, L-menthyl, 3,3,5-trimethylcyclohexanyl, 3,3,5-trimethylhexanyl, dodecyl, hexadecyl, 2-butyloctyl, 2-hexyldecyl, octadecyl, 3,7-dimethyloctyl, 1,3,3-trimethylbicyclo[2.2.1]heptan-2-yl, unsubstituted or at least mono-substituted benzyl radical or an unsubstituted or at least mono-substituted phenyl radical;

R₁₀ represents methyl, ethyl, n-propyl, n-butyl, tert-butyl or phenyl;

R₁₂, R₁₃, R₁₄, R₁₅ and R₁₆, independent of each other, each represents methyl, ethyl, —O—CH₃, —O—CH₂—CH₃ or phenyl;

R₁₇ represents methyl, ethyl, —O—CH₃, —O—CH₂—CH₃ or phenyl;

R₁₈ represents a hydrogen atom, —OH, methyl, —O—CH₃, —O(C═O)—CH₃, —O(C═O)—CH₂—CH₃, or —O(C═O)—CH₂—CH₂—CH₃;

R₁₉ represents a hydrogen atom, —OH, —O(C═O)—CH₃, —O(C═O)—CH₂—CH₃, —O(C═O)—CH₂—CH₂—CH₃, —O—CH₃, a saturated or unsaturated, linear or branched —O—CH₃, —O—CH₂—CH₃, —O—CH₂—CH₂—CH₃, —O—CH₂—CH₂—CH₂—CH₃, —O-phenyl or a 2-ethylhexyloxy radical which is unsubstituted or at least mono-substituted with a —SO₃M or a —N(R₁₁)₃⁺ radical, with M being H, K⁺ or Na⁺;

R₂₀ and R₂₁, identical or different, each represents a hydrogen atom, —OH, —O—CH₃, —O—CH₂—CH₃, —O—CH₂—CH₂—CH₃, —O—CH₂—CH₂—CH₂—CH₃, —O-phenyl or a 2-ethylhexyloxy radical which is unsubstituted or at least mono-substituted with at least one —SO₃M group, with M being H, K⁺ or Na⁺;

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (YA)

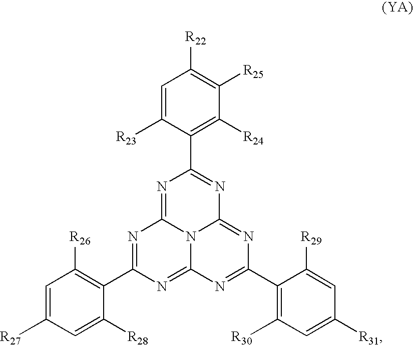

wherein

R₂₂, R₂₃, R₂₄, R₂₅, R₂₆, R₂₇, R₂₈, R₂₉, R₃₀ and R₃, have the following meaning:

| R₂₂ | R₂₃ | R₂₄ | R₂₅ | R₂₆ | R₂₇ | R₂₈ | R₂₉ | R₃₀ | R₃₁ |
|---|---|---|---|---|---|---|---|---|---|
| —O—CH₃ | H | H | H | —OH | 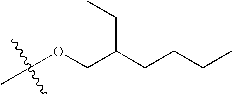 | H | —OH | H | 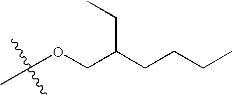 |
| 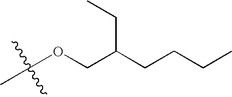 | —OH | H | H | —CH₃ | —CH₃ | H | —CH₃ | H | —CH₃ |
| 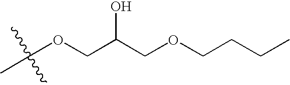 | —OH | H | H | —CH₃ | —CH₃ | H | —CH₃ | H | —CH₃ |
| 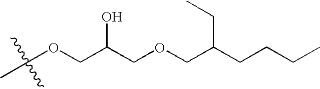 | —OH | H | H | —CH₃ | —CH₃ | H | —CH₃ | H | —CH₃ |
| 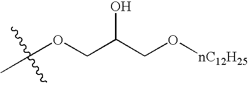 | —OH | H | H | —CH₃ | —CH₃ | H | —CH₃ | H | —CH₃ |
| 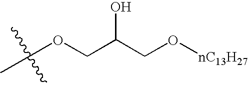 | —OH | H | H | —CH₃ | —CH₃ | H | —CH₃ | H | —CH₃ |
| 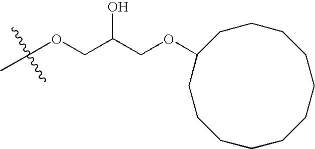 | —OH | H | H | —CH₃ | —CH₃ | H | —CH₃ | H | —CH₃ |
| 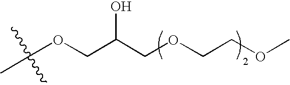 | —OH | H | H | —CH₃ | —CH₃ | H | —CH₃ | H | —CH₃ |
| 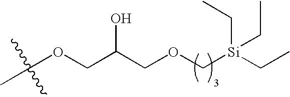 | —OH | H | H | —CH₃ | —CH₃ | H | —CH₃ | H | —CH₃ |
| 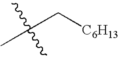 | H | H | H | H | H | H | H | H | H |
| —O—Ac* | —O—Ac | H | H | H | H | H | H | H | H |
| 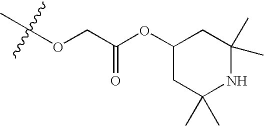 | H | H | H | —CH₃ | —CH₃ | H | —CH₃ | H | —CH₃ |
| 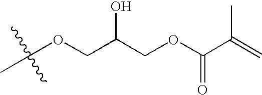 | H | H | H | —CH₃ | —CH₃ | H | —CH₃ | H | —CH₃ |
| —O—CH₃ | —OH | H | H | H | -Ph** | H | H | H | -Ph |
| —O-Bu*** | —OH | H | H | H | -Ph | H | H | H | -Ph |
| 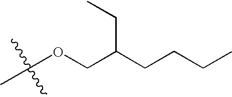 | —OH | H | H | H | -Ph | H | H | H | -Ph |
| 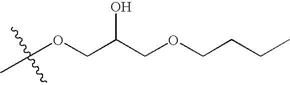 | —OH | H | H | H | -Ph | H | H | H | -Ph |
| 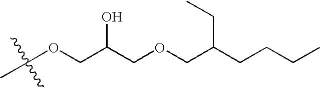 | —OH | H | H | H | -Ph | H | H | H | -Ph |
| 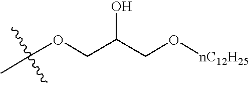 | —OH | H | H | H | -Ph | H | H | H | -Ph |
| 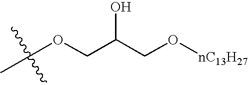 | —OH | H | H | H | -Ph | H | H | H | -Ph |
| 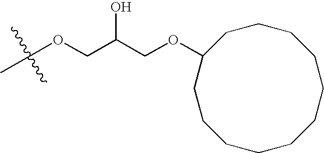 | —OH | H | H | H | -Ph | H | H | H | -Ph |
| 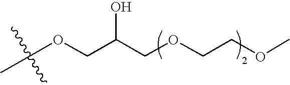 | —OH | H | H | H | -Ph | H | H | H | -Ph |
| 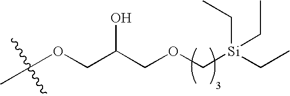 | —OH | H | H | H | -Ph | H | H | H | -Ph |
| 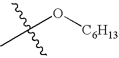 | H | H | H | H | -Ph | H | H | H | -Ph |
| 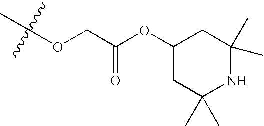 | H | H | H | H | -Ph | H | H | H | -Ph |
| 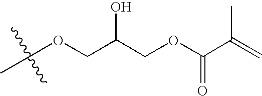 | H | H | H | H | -Ph | H | H | H | -Ph |
| H | H | H | H | H | H | H | H | H | H |
| H | —O—CH₃ | H | H | H | H | H | H | H | H |
| —O—CH₃ | H | H | H | H | H | H | H | H | H |
| —O—CH₃ | —OH | H | H | H | H | H | H | H | H |
| —O—CH₃ | —OH | H | H | H | —CH₃ | H | H | H | —CH₃ |
| —O—CH₃ | —OH | H | H | —CH₃ | —CH₃ | H | —CH₃ | H | —CH₃ |
| —O—CH₃ | —OH | H | H | —O—CH₃ | —O—CH₃ | H | —O—CH₃ | H | —O—CH₃ |
| —OC₆H₁₃ | —OH | H | H | —CH₃ | —CH₃ | H | —CH₃ | H | —CH₃ |
| —OC₆H₁₃ | —O—CH₃ | H | H | H | H | H | H | H | H |
| —OC₆H₁₃ | —OH | H | H | H | —O—CH₃ | H | H | H | —O—CH₃ |
| —OC₆H₁₃ | —OH | H | H | —CH₃ | —CH₃ | —CH₃ | —CH₃ | —CH₃ | —CH₃ |
| —OC₆H₁₃ | —O—CH₃ | H | H | —CH₃ | —CH₃ | —CH₃ | —CH₃ | —CH₃ | —CH₃ |
| —OC₆H₁₃ | —OH | H | —CH₃ | H | H | H | H | H | |
| —OC₆H₁₃ | —OH | —CH₃ | H | H | H | H | H | H | H |
| 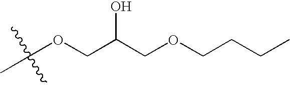 | —OH | H | H | —OH | 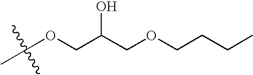 | H | H | H | H |
| 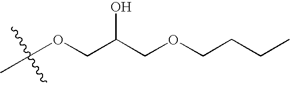 | —OH | H | H | —OH | 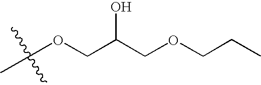 | H | —CH₃ | H | —CH₃ |
| 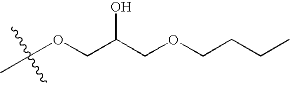 | —OH | H | H | —OH | 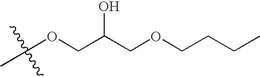 | H | —CH₃ | —CH₃ | —CH₃ |
| —O—CH₃ | —OH | H | H | —OH | 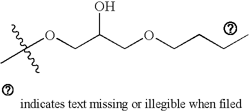 | H | —OH | H | 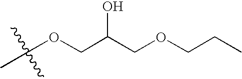 |
| 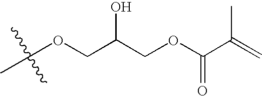 | —OH | H | H | —CH₃ | —CH₃ | H | —CH | H | —CH₃ |
| —OC₁₂H₂₅ | —OH | H | H | —CH₃ | —CH₃ | H | —CH₃ | H | —CH₃ |
| —O—CH₃ | —O—CH₃ | H | H | H | —CH₃ | H | H | H | —CH₃ |
| —O—CH₃ | —O—CH₃ | H | H | —CH₃ | —CH₃ | H | —CH₃ | H | —CH₃ |
| —OC₁₂H₂₅ | —O—CH₃ | H | H | H | H | H | H | H | H |
| —OC₁₂H₂₅ | —O—CH₃ | H | H | H | —CH₃ | H | H | H | —CH₃ |
| —OC₁₂H₂₅ | —O—CH₃ | H | H | —CH₃ | —CH₃ | H | —CH₃ | H | —CH₃ |
| H | H | H | H | —OH | 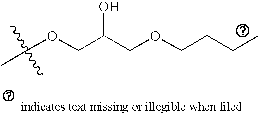 | H | —OH | H | 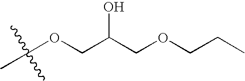 |
| —O—CH₃ | —O—CH₃ | H | H | H | H | H | H | H | H |
| —OC₁₂H₂₅ | —O—CH₃ | H | H | H | H | H | H | H | H |
| —O—CH₃ | —O—CH₃ | H | H | H | —CH₃ | H | H | H | —CH₃ |
| —OC₆H₁₃ | —O—CH₃ | H | H | H | —CH₃ | H | H | H | —CH₃ |
| —O—CH₃ | —O—CH₃ | H | H | —CH₃ | —CH₃ | H | H | H | —CH₃ |
| —OC₁₂H₂₅ | —O—CH₃ | H | H | —CH₃ | —CH₃ | H | —CH₃ | H | —CH₃ |
| H | —O—CH₃ | H | H | H | H | H | —CH₃ | H | H |
| —O—CH₃ | H | H | H | H | H | H | H | H | H |
| —O—CH₃ | H | H | H | H | —O—CH₃ | H | H | H | H |
| —O—CH₃ | H | H | H | H | —O—CH₃ | H | H | H | —O—CH₃ |
| H | —OH | H | H | H | H | H | H | H | H |
| H | —OH | H | H | —OH | H | H | H | H | H |
| H | H | H | H | H | H | H | H | H | H |
| 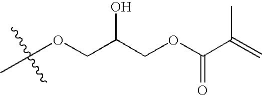 | —OH | H | H | —CH₃ | —CH₃ | H | —CH₃ | H | —CH₃e |
| —O—CH₃ | —OH | H | H | —O—CH₃ | —O—CH₃ | H | —O—CH₃ | H | —O—CH₃ |
| —O—CH₃ | —OH | H | H | —OH | —O—CH₃ | H | —O—CH₃ | H | —O—CH₃ |
| —O—CH₃ | —OH | H | H | H | H | H | H | H | H |
| 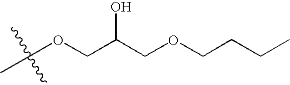 | —OH | H | H | —OH | 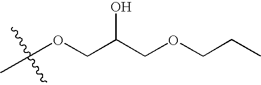 | H | H | H | H |

*-Ph stands for a phenyl radical,
**—O-but stand for an —O-butyl radical and
***—O—Ac stand for an —O-acyl radical;

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (YB)

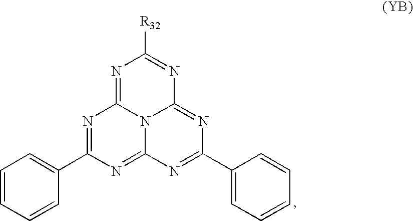

wherein

R₃₂ represents a radical selected from the group consisting of

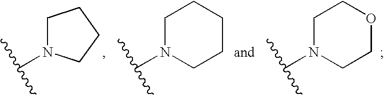

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (YC)

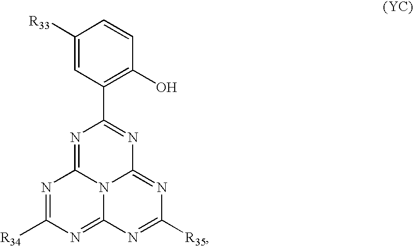

wherein R₃₃, R₃₄ and R₃₅ have the following meaning:

| R₃₃ | R₃₄ | R₃₅ |
|---|---|---|
| H | —O—CH₃ | —O—CH₃ |
| —CH₃ | —O—CH₃ | —O—CH₃ |
| —CH₃ | —O—CH₃ | —N(Me)₂ |
| —CH₃ | —N(Me)₂ | —N(Me)₂ |

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (YD)

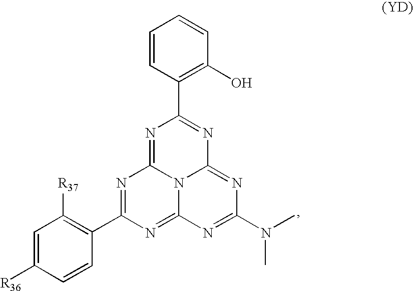

wherein

R₃₆ and R₃₇ have the following meaning:

| R₃₆ | R₃₇ |
|---|---|
| H | H |
| H | —OH |
| —CH₃ | H |

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is a heptaazaphenalene compound of formula (YE)

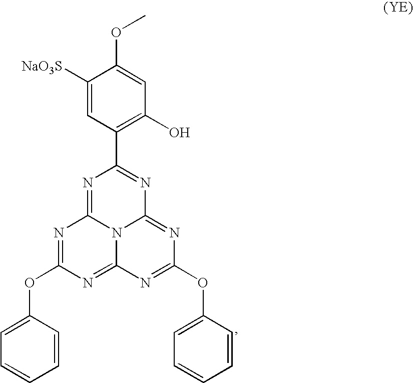

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (YF)

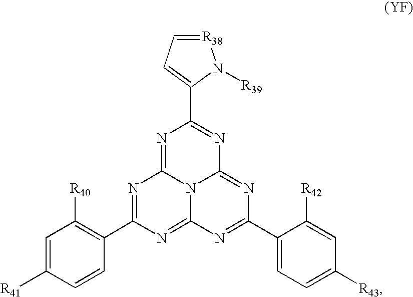

wherein

R₃₈, R₃₉, R₄₀, R₄₁, R₄₂ and R₄₃ have the following meaning:

| R₃₈ | R₃₉ | R₄₀ | R₄₁ | R₄₂ | R₄₃ |
|---|---|---|---|---|---|
| N | —CH₃ | —OH | —OH | —OH | —OH |
| N | —CH₃ | —OH | 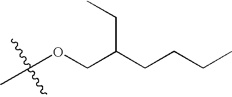 | —OH | 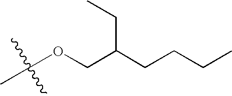 |
| N | —CH₃ | 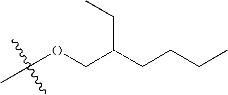 | 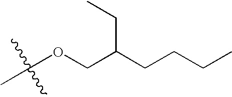 | —OH | 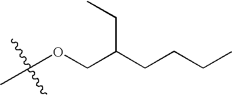 |
| N | -Ph* | —OH | —OH | —OH | —OH |
| N | -Ph | —OH | 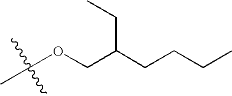 | —OH | 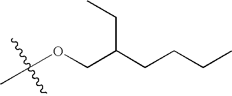 |
| —CH | —CH₂-Ph** | —OH | —OH | —OH | —OH |
| —CH | —CH-Ph | —OH | 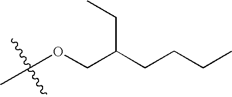 | —OH | 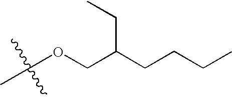 |

*-Ph stands for a phenyl radical,
—CH₂-Ph** stand for a —CH₂-phenyl radical;

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound of general formula (YG)

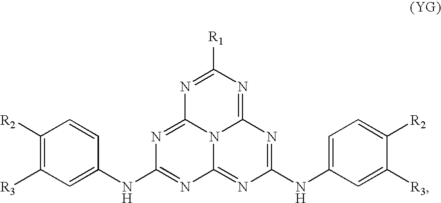

wherein
R₄₄, R₄₅ and R₄₆ have the following meaning:

| R₄₄ | R₄₅ | R₄₆ |
|---|---|---|
| 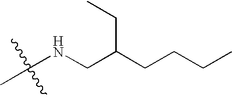 | 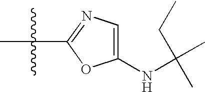 | H |
| 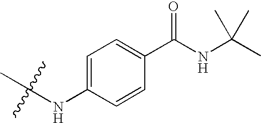 | 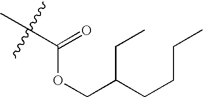 | H |
| 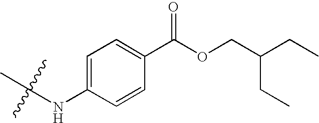 | 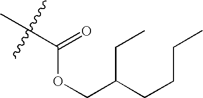 | H |
| 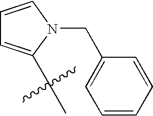 | -Ph | H |
| 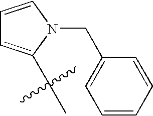 | 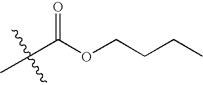 | H |
| 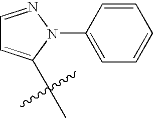 | 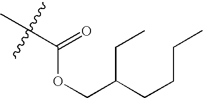 | H |
| 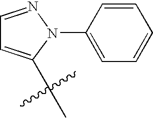 | 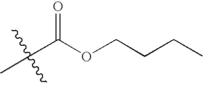 | H |
| 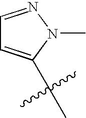 | 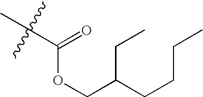 | H |
| 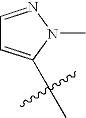 | 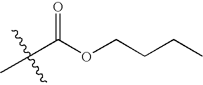 | H |
| 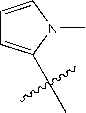 | 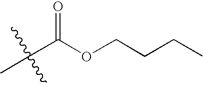 | H |
| 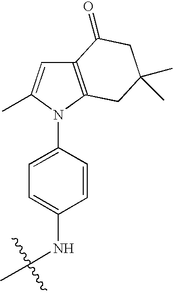 | 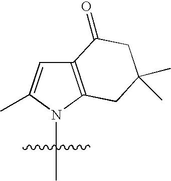 | H |
| 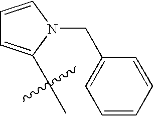 | —C—O-Ph* | H |

*—C—O-Ph stands for a —C—O-phenyl radical;

preferably with the aforementioned provisos;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

According to a further preferred embodiment of the present invention, the UV-absorbing active substance (A) is at least one heptaazaphenalene compound selected from the group comprising
2,5,8-tris-(4-(butoxycarbonyl)phenylamino)-1,3,4,6,7,9,9b-heptaazaphenalene,
2,5,8-tris-(4-(2-ethylhexyloxycarbonyl)phenylamino)-1,3,4,6,7,9,9b-heptaazaphenalene,
2,5,8-tris-(4-(imidazo[1,2-a]pyridin-2-yl)-phenylamino)-1,3,4,6,7,9,9b-heptaazaphenalene,
2,5,8-tris-(4-(4,5,6,7-tetrahydro-2,6,6-trimethyl-4-oxo indol-1-yl)phenylamine)-1,3,4,6,7,9,9b-heptaazaphenalene,
2,5,8-tris-(biphenyl-4-ylamino)-1,3,4,6,7,9,9b-heptaazaphenalene,
2,5,8-tris-(4-(1H-benzo[d]imidazol-2-yl)phenylamino)-1,3,4,6,7,9,9b-heptaazaphenalene,
2,5-bis-(biphenyl-4-ylamino)-8-(4-(butoxycarbonyl)phenylamino)-1,3,4,6,7,9,9b-heptaazaphenalene,
2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene,
2,5,8-tris-(2,4-dihydroxyphenyl)-1,3,4,6,7,9,9b-heptaazaphenalene,
2,5-dichloro-8-(1-methyl-1H-pyrrole-2-yl)-1,3,4,6,7,9,9b-heptaazaphenalene,
2,5-bis-(4-(butoxycarbonyl)phenylamino)-8-(2-(1-methyl-1H-pyrrole-2-yl)-1,3,4,6,7,9,9b-heptaazaphenalene,
2-(4-(carboxy)phenylamino)-5,8-bis-(4-methylphenyl)-1,3,4,6,7,9,9b-heptaazaphenalene,
2,5,8-tris-(4-(4,5,6,7-tetrahydro-2,6,6-trimethyl-4-oxo indol-1-yl)phenylamine)-1,3,4,6,7,9,9b-heptaazaphenalene,
2,5-bis-(4-(butoxycarbonyl)phenylamino)-8-(1-methyl-1H-pyrrole-2-yl)-1,3,4,6,7,9,9b-heptaazaphenalene,
2,5-bis(2,4-dihydroxyphenyl)-8-(1-methyl-1H-pyrazol-5-yl)-1,3,4,6,7,9,9b-heptaazaphenalene,
2,5-bis{[4-(2-ethylhexyloxy)-2-hydroxy]phenyl}-6-(1-methyl-1H-pyrazol-5-yl)-1,3,4,6,7,9,9b-heptaazaphenalene,
2-[2,4-bis(2-ethylhexyloxy)phenyl]-5-[4-(2-ethylhexyl oxy)-2-hydroxyphenyl]-8-(1-methyl-1H-pyrazol-5-yl)-1,3,4,6,7,9,9b-heptaazaphenalene,
2,5-bis(2,4-dihydroxyphenyl)-8-(1-phenyl-1H-pyrazol-5-yl)-1,3,4,6,7,9,9b-heptaazaphenalene,
2,5-bis{[4-(2-ethylhexyloxy)-2-hydroxy]phenyl}-8-(1-phenyl-1H-pyrazol-5-yl)-1,3,4,6,7,9,9b-heptaazaphenalene,
2,5-bis[4-(butoxycarbonyl)phenylamino]-8-(1-methyl-1H-pyrazol-5-yl)-1,3,4,6,7,9,9b-heptaazaphenalene,
2,5-bis{4-[(2-(ethylhexyloxy)carbonyl]phenylamino}-8-(1-methyl-1H-pyrazol-5-yl)-1,3,4,6,7,9,9b-heptaazaphenalene,
2,5-bis[4-(butoxycarbonyl)phenylamino]-8-(1-phenyl-1H-pyrazol-5-yl)-1,3,4,6,7,9,9b-heptaazaphenalene,
2,5-bis{4-[(2-ethylhexyloxy)carbonyl]phenylamino}-8-(1-phenyl-1H-pyrazol-5-yl)-1,3,4,6,7,9,9b-heptaazaphenalene,
2-(1-benzyl-1H-pyrrole-2-yl)-5,8-bis(2,4-dihydroxyphenyl)-1,3,4,6,7,9,9b-heptaazaphenalene,
2-(1-benzyl-1H-pyrrole-2-yl)-5,8-bis[4-(2-ethylhexyloxy)-2-hydroxyphenyl]-1,3,4,6,7,9,9b-heptaazaphenalene,
2-(1-benzyl-1H-pyrrole-2-yl)-5,8-bis[4-(butoxycarbonyl)phenylamino]-1,3,4,6,7,9,9b-heptaazaphenalene,
2-(1-benzyl-1H-pyrrole-2-yl)-5,8-bis(biphenyl-4-ylamino)-1,3,4,6,7,9,9b-heptaazaphenalene,
2-(1-benzyl-1H-pyrrole-2-yl)-5,8-bis(4-benzoylphenylamino)-1,3,4,6,7,9,9b-heptaazaphenalene,
2-(1-benzyl-1H-pyrrole-2-yl)-5,8-bis(9-oxo-9H-fluoren-3-ylamino)-1,3,4,6,7,9,9b-heptaazaphenalene,
2-(4-(tert-butylcarbamoyl)phenylamino)-5,8-bis-(4-(2-ethylhexyloxycarbonyl)phenylamino)-1,3,4,6,7,9,9b-heptaazaphenalene,
2,5-bis-[(4-(2-ethylhexyloxy)-2-hydroxy)-phenyl]-8-(4-methoxyphenyl)-1,3,4,6,7,9,9b-heptaazaphenalene,
2-(2-ethylhexylamino)-5,8-bis-(4-(5-(1,1-dimethylpropyl)benzo[d]oxazol-2-yl)phenylamino)-1,3,4,6,7,9,9b-heptaazaphenalene,
2,5,8-tris-(4-(1H-pyrazol-1-yl)phenylamino)-1,3,4,6,7,9,9b-heptaazaphenalene
2,5,8-tris-(4-benzoylphenylamino)-1,3,4,6,7,9,9b-heptaazaphenalene,
2,5,8-tris-(4-butoxy-2-hydroxyphenyl)-1,3,4,6,7,9,9b-heptaazaphenalene,
2,5,8-tris-(naphthalen-2-ylamino)-1,3,4,6,7,9,9b-heptaazaphenalene,
2,5-bis-(4-(butoxycarbonyl)phenylamino)-8-(2-(1-methyl-1H-indol-3-yl)-1,3,4,6,7,9,9b-heptaazaphenalene,
2,5,8-tris-(biphenyl-4-yloxy)-1,3,4,6,7,9,9b-heptaazaphenalene,
2,5,8-tris-(3-methoxyphenylamino)-1,3,4,6,7,9,9b-heptaazaphenalene,
2,5,8-tris-(4-methoxyphenylamino)-1,3,4,6,7,9,9b-heptaazaphenalene,
2,5,8-tris-(4-((E)-3-ethoxy-3-oxoprop-1-enyl)phenylamino)-1,3,4,6,7,9,9b-heptaazaphenalene,
2,5,8-tris-(methoxycarbonyl-4′-biphenyl-4-ylamino)-1,3,4,6,7,9,9b-heptaazaphenalene,
2,5,8-tris-(4-(methoxycarbonyl)phenylamino)-1,3,4,6,7,9,9b-heptaazaphenalene,
2,5,8-tris-(4-(1H-benzo[d]imidazol-2-yl)-3-hydroxyphenylamino)-1,3,4,6,7,9,9b-heptaazaphenalene,
2,5,8-tris-(4-(phenylamino)phenylamino)-1,3,4,6,7,9,9b-heptaazaphenalene,
2,5,8-tris-((4-(E)-styrylphenyl)amino)-1,3,4,6,7,9,9b-heptaazaphenalene,
2,5,8-tris-(2-ethylhexylamino)-1,3,4,6,7,9,9b-heptaazaphenalene,
2,5,8-tris-(4-(L-menthylcarbonyl)phenylamino)-1,3,4,6,7,9,9b-heptaazaphenalene,
2,5,8-tris-(4-((3,3,5-trimethylcyclohexyloxy)carbonyl)phenylamino)-1,3,4,6,7,9,9b-heptaazaphenalene,
2,5,8-tris-(4-((E)-3-(2-ethylhexyloxy)-3-oxoprop-1-enyl)phenylamino)-1,3,4,6,7,9,9b-heptaazaphenalene,
2-(3-((E)-3-(2-ethylhexyloxy)-3-oxoprop-1-enyl)phenylamino)-5,8-bis-(4-((E)-3-(2-ethylhexyloxy)-3-oxoprop-1-enyl)phenylamino)-1,3,4,6,7,9,9b-heptaazaphenalene,
2,5,8-tris-(3-nitrophenylamino)-1,3,4,6,7,9,9b-heptaazaphenalene,
2,5,8-tris-(4-(2-butyloctyloxycarbonyl)phenylamino)-1,3,4,6,7,9,9b-heptaazaphenalene,
2,5,8-tris-(4-(2-hexyldecyloxycarbonyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(hexyldecyloxycarbonyl)phenylamino)-1,3,4,6,7,9,9b-heptaazaphenalene,
2,5,8-tris-(4-(octyldecyloxycarbonyl)phenylamino)-1,3,4,6,7,9,9b-heptaazaphenalene,
2,5,8-tris-((3-(E)-styrylphenyl)amino)-1,3,4,6,7,9,9b-heptaazaphenalene,
2,5,8-tris-(4-((3,5,5-trimethylhexyloxy)carbonylphenylamino)-1,3,4,6,7,9,9b-heptaazaphenalene,
2,5-bis-(3-(methoxy)phenylamino)-8-(2-(1-methyl-1H-pyrrole-2-yl)-1,3,4,6,7,9,9b-heptaazaphenalene,
2,5,8-tris-(4-((2-ethylhexyl)carbamoyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(1,3,3-trimethylbicyclo[2.2.1]heptan-2-yloxy)phenylamino)-1,3,4,6,7,9,9b-heptaazaphenalene,
2,5,8-tris-(1H-indol-5-ylamino)-1,3,4,6,7,9,9b-heptaazaphenalene,
2,5,8-tris-(4-((3,7-dimethyloctyloxy)carbonyl)phenylamino)-1,3,4,6,7,9,9b-heptaazaphenalene and
2,5,8-tris-(2-amino-4,5-dimethylphenylamino)-1,3,4,6,7,9,9b-heptaazaphenalene;
   preferably with the aforementioned provisos;
   and/or at least one of the stereoisomers, preferably enantiomers or diastereomers, a racemate or a mixture of at least two of its stereoisomers, preferably enantiomers and/or diastereomers in any mixing ratio, or at least one physiologically acceptable salt, or at least one corresponding solvate of the aforementioned compounds;
   which active substance (A) has its absorption maximum λ_{max(A)} in the range of either UV-A or UV-B radiation
   and the UV-absorbing active substance (B)
   is at least one compound, which has its absorption maximum λ_{max(B)} either in the range of UV-A or UV-B radiation, preferably with the proviso that λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

It has been surprisingly found, that an efficient protection against UV-A or UV-B as well as UV-A and UV-B radiation is provided, if the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1, more preferably 4:10 to 7:1, most preferably from 2:8 to 8:1.

The absorption maximum λₘₐₓ according to the present invention is defined as the wavelength, at which an UV-absorbing active substance has its absorption maximum. The relevant wavelength range in which the inventive combination absorbs is preferably in the range of UV-A and/or UV-B radiation, i.e. from 290 to 400 nm. The UV-absorbing active substance (A) should have at least one λ_{max(A)} in either the UV-A or UV-B range, preferably with the proviso defined above, and regardless of any additional absorption capacity in the UV radiation range, wherein the λ_{max(A)} does not lie.

It is also preferred that the absorption maxima λ_{max(A)} and λ_{max(B)}, respectively, at least differ in 10 nm, more preferably in at least 20 nm and most preferably in at least 40 nm to each other. For example, if the absorption maximum λ_{max(A)} of UV-absorbing active substance (A) corresponds to 310 nm [λ_{max(A)}=³¹ 0 nm], the absorption maximum λ_{max(B)} of UV-absorbing active substance (B) is preferably <300 nm [λ_{max(B)}<³⁰⁰ nm] or >320 nm [λ_{max(B)}>³²⁰ nm].

The term “absorption” according to the present invention means any of the following means of reducing the exposure to UV radiation like absorbing, extincting, filtering, scattering, deflecting or reflecting of radiation, since absorption only means the ratio of transmitted radiation to the incident radiation, without determining the mechanism according to which the radiation is reduced.

The molar extinction coefficient (ε) of a substance is usually measured in solution and can be calculated by the following equation (E 1),

$$ɛ = \frac{1}{cl} {log}_{10} \frac{Io}{I} , \qquad ( E 1 )$$

wherein “I” means the intensity of radiation transmitted through the sample solution with the dissolved substance, “I₀” is the intensity of radiation transmitted through a reference sample consisting of the same solvent but without such substance, “c” is the molar concentration of the substance in moles/liter, and “I” is the path length through the solution in centimeters (usually 1 cm). Under defined conditions with regard to the solvent, pH and temperature the molar extinction coefficient of a particular compound is a constant at a specified wavelength. Common synonyms for the molar extinction coefficient of a substance are molar absorptivity or molar absorption coefficient.

The molar extinction coefficient ε_{λmax(A)} of the UV-absorbing active substance (A) according to the present invention defines the molar extinction coefficient at the absorption maximum λₘₐₓ of UV-absorbing active substance (A). The molar extinction coefficient Γ_{λmax(B)} of the UV-absorbing active substance (B) according to the present invention defines the molar extinction coefficient at the absorption maximum λₘₐₓ of UV-absorbing active substance (B).

According to the present invention, the UV-absorbing active substance (B) has its absorption maximum λ_{max(B)} in the range of 290 to 400 nm, preferably with the proviso that λ_{max(B)} of active substance (B) does not lie in the same UV radiation range wherein the λ_{max(A)} of active substance (A) lies. It is also preferred that the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1.

Such UV-absorbing active substance (B) is preferably at least one organic and/or at least one inorganic compound known in the art.

Preferably, such UV-absorbing active substance (B) is at least one compound selected from the group comprising substituted acrylic acid derivatives, allantoin derivatives, aminobenzoic acid derivatives, aminobenzamides derivatives, anthranilic acid derivatives, benzalmalonic acid derivatives, benzimidazole derivatives, benzoic acid derivatives, benzophenone derivatives, benzothiazole derivatives, benzotriazole derivatives, benzoxazole derivatives, camphor derivatives, cinnamic acid derivatives, coumarinic acid derivatives, curcumin derivatives, dianisoylmethane derivatives, dibenzalazine derivatives, dibenzoylmethane derivatives, dioxane derivatives, fungi extract, ferulic acid derivatives, furane derivatives, glutamic acid derivatives, imidazoline derivatives, metal oxides, metal dioxides, nicotinic acid derivatives, nitrobenzamides derivatives, nitrobenzoic acid derivatives, nucleic acid derivatives, p-aminobenzoic acid (PABA) derivatives, plant extracts, propenoate derivatives, pyrimidine derivatives, salicylic acid derivatives, tetrazole derivatives, triazine derivatives, tributamine derivatives, urocanic acid derivatives and vitamin B₆ derivatives;

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned substances;
which UV-absorbing active substance (B) has its absorption maximum λ_{max(B)} in the range of 290 to 400 nm, preferably with the proviso that λ_{max(B)} of active substance (B) does not lie in the same UV radiation range wherein the λ_{max(A)} of active substance (A) lies.

The aforementioned active substances (B) are known to those skilled in the art and are either obtainable by standard techniques or are commercially available.

The term “derivative”, as defined in the present invention, refers to substances in form of their respective free bases, acids, salts, esters and/or ethers.

In a further preferred embodiment of the present invention, the combination according to the present invention comprises as UV-absorbing active substance (B) at least one compound of general formula (AA)

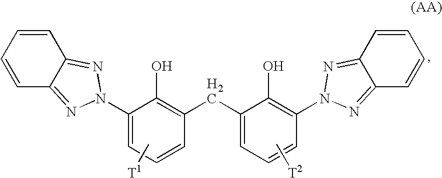

wherein

T¹ and T², independent of each other, each represents a C₁₋₈ alkyl radical which can be unsubstituted or at least mono-substituted with radicals independently selected from the group consisting of C₁₋₄ alkyl, C₅₋₁₂ cycloalkyl and aryl;

and/or
at least one compound of general formula (AB)

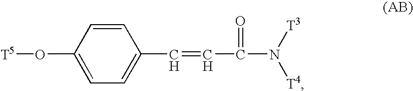

wherein

T³ represents a —(CONH)ₛ-phenyl moiety, in which s is 0 or 1 and the phenyl group is unsubstituted or at least mono-substituted with radicals independently selected from the group consisting of —OH, C₁₋₁₈ alkyl and —O—C₁₋₈ alkyl, or a —C(═O)—OT^{3′} moiety, in which T^{3′} is a C₁₋₁₈ alkyl radical;

T⁴ represents a hydrogen atom or a C₁₋₄ alkyl radical; and

T⁵ represents H or a linear or branched —C₁₋₁₈ alkyl radical;

and/or
at least one compound of general formula (AC)

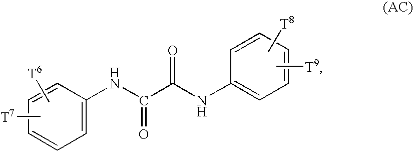

wherein

T⁶, T⁷, T⁸ and T⁹, independent of each other, each represents a C₁₋₈ alkyl radical or an —O—C₁₋₈ alkyl radical;

and/or
at least one compound of general formula (AD)

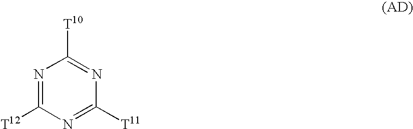

wherein

T¹⁰, T¹¹ and T¹², independent of each other, each represents a radical selected from the group consisting of —NR^{T10}R^{T11}, phenyl, —O-phenyl or pyrrolyl, which is unsubstituted or at least mono-substituted with 1, 2 or 3 substituent(s) independently selected from the group consisting of —OH, —C₁₋₁₈ alkyl, —O—C₁₋₁₈ alkyl, —(C═O)—C₁₋₁₈ alkyl, C₅₋₈ cycloalkyl, a methylidenecamphor group or a —(CH═CH)ₙ(CO)—OT^{10′}, with T^{10′} being either C₁₋₁₈ alkyl or cinnamyl, and n is 0 or 1;

wherein R^{T10} and R^{T11}, independent of each other, each represents a hydrogen atom; a linear or branched, unsubstituted or at least unsubstituted C₁₋₁₈ alkyl radical; an unsubstituted or at least unsubstituted aryl radical or an unsubstituted or at least unsubstituted —(C═O)—C₁₋₁₈ alkyl radical;
and/or
at least one compound of general formula (AE)

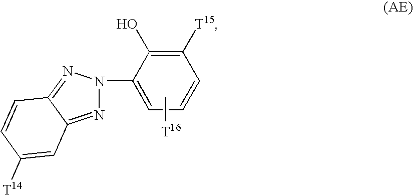

wherein

T¹⁴ represents a hydrogen atom or a C₁₋₁₈ alkyl radical;

T¹⁵ represents a C₁₋₈ alkyl radical which can be unsubstituted, an at least mono-substituted with a phenyl radical or a radical of general formula (VI′)

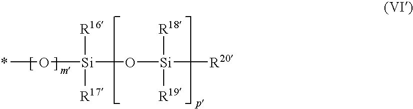

wherein
m′=0 or 1;
p′=0, 1, 2, 3 or 4;

R^{16′}, R^{17′}, R^{18′}, R^{19′} and R^{20′}, independent of each other, each represents an unsubstituted or at least mono-substituted C₁₋₆ alkyl radical; an unsubstituted or at least mono-substituted —O—C₁₋₆ radical; an unsubstituted or at least mono-substituted aryl radical; or an —O—Si(R^{21′})₃ radical with R^{21′} representing an unsubstituted or at least mono-substituted C₁₋₆ alkyl radical; an —O—C₁₋₆ radical; an unsubstituted or at least mono-substituted aryl radical;

T¹⁶ represents a C₁₋₈ alkyl radical, which can be unsubstituted or an at least mono-substituted with a phenyl radical;

and/or
at least one compound of general formulae (AF), (AG) and (AH)

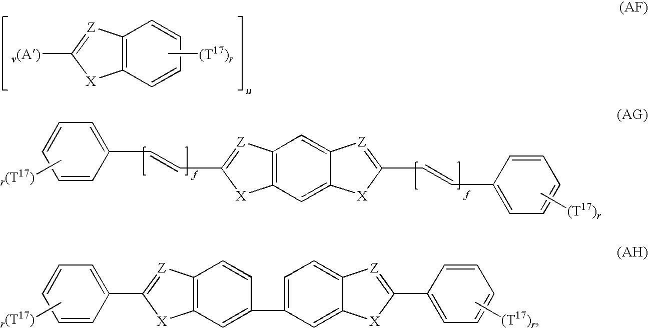

wherein
each of the symbols X, independently from each other represents an oxygen or sulfur atom or a group NT^{17′}, each of the symbols Z, independently from each other represents a nitrogen atom or a CH group, each of the symbols T¹⁷, independently from each other represents an —OH group, a halogen atom, a linear or branched C₁₋₈ alkyl radical optionally containing a silicon atom, or a linear or branched —O—C₁₋₈ alkyl radical, each of the numbers r is independently from each other 0, 1 or 2, u represents an integer ranging from 1 to 4 inclusive, v is equal to 0 or 1, each of the numbers r is independently from each other equal to 0 or 1, each of the symbols T^{17′} independently represents a hydrogen atom or a linear or branched C₁₋₈ alkyl radical or benzyl group optionally containing a silicon atom;

A′ represents a radical of valency u selected from the following group of formulae:

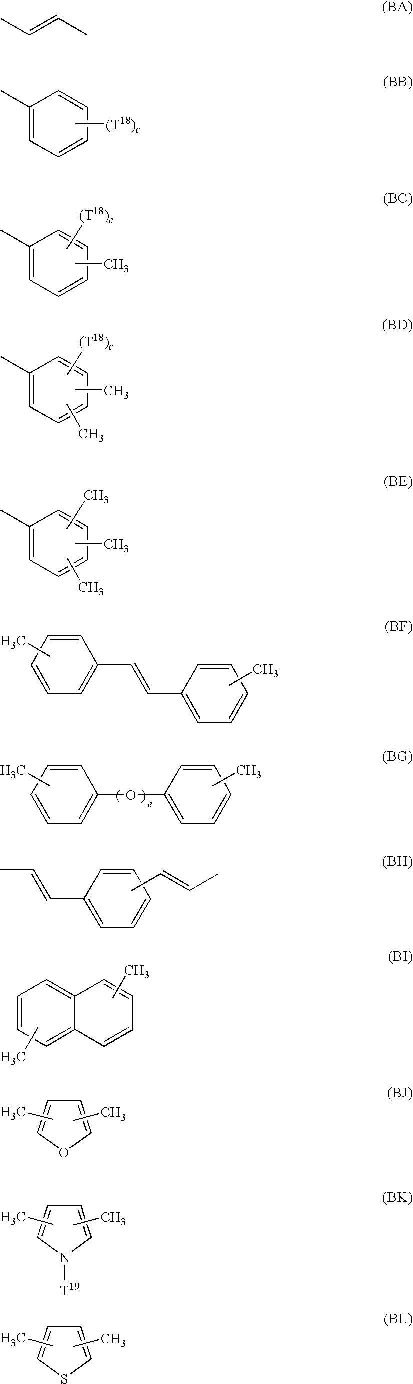

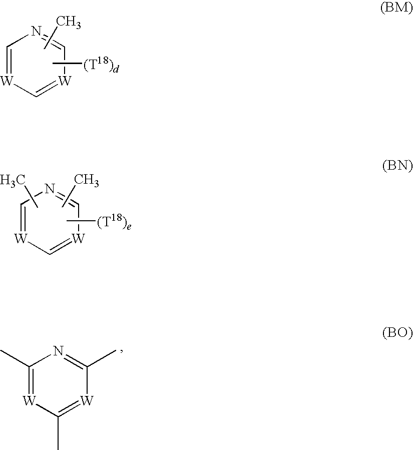

wherein
each of the symbols T¹⁸, independently from each other represents a halogen atom or a linear or branched C₁₋₄ alkyl or —O—C₁₋₄ alkyl radical, or —OH; T¹⁹ represents a hydrogen atom or a linear or branched C₁₋₄ alkyl radical; W represents a —CH— group or a nitrogen atom; c=0, 1, 2, 3 or 4; d=0, 1 or 2 and e=0 or 1;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds of general formulae (AA) to (AH);
wherein the UV-absorbing active substance (B) has its absorption maximum λ_{max(B)} in the range of 290 to 400 nm, preferably with the proviso that λ_{max(B)} of active substance (B) does not lie in the same UV radiation range wherein the λ_{max(A)} of active substance (A) lies.

In a further preferred embodiment of the present invention the active substance (B) is at least one compound selected from the group comprising 2-ethylhexyl 2-cyano-3,3′-diphenylacrylate, ethyl 2-cyano-3,3′-diphenylacrylate, polymer of N-{(2 and 4)[(2-oxoborn-3-ylidene)methyl]benzyl}acrylamide, methyl 4-aminobenzoate, butyl 4-aminobenzoate, 5-methyl-2-(1-methylethyl)-cyclohexyl 2-aminobenzoate, glyceryl aminobenzoate, 2-ethylhexyl-p-dimethylethyl-aminobenzoate, 4-aminobenzamide, homomethyl-N-acetyl-anthranilic acid, menthyl antrhanilate, 2-(1H-benzimidazol-2-yl)-4-methoxyphenol, 2,2′-bis(benzimidazole), monosodium salt of 2-2′-bis-(1,4-phenylene)1H-benzimidazole-4,6-disulphonic acid, 2-phenylbenzimidazole-5-sulphonic acid, 5,5′,6,6′-tetramethyl-2,2′-bis(benzimidazole), 5,5′-dimethyl-2,2′bis(benzimidazole), 6-methoxy-2,2′-bis(benzimidazole), 1,4-phenylenebis(2-benzimidazolyl), 1,2-phenylenebis)benzimidazolyl), 1,4-phenylenebis(N-2-ethylhexyl-2-benzimidazolyl), 1,4-phenylenebis(N-tri-methylsilylmethyl-2-benzimidazolyl), 2-(1H-benzimidazol-2-yl)benzothiazole, 2-(1H-benzimidazol-2-yl)benzoxazole, N,N′-dimethyl-2,2′-bis(benzimidazole), phenylbenzimidazole-5-sulfonic acid (Ensulizole) and its salts, 2-2′-bis-(1,4-phenylene)1H-benzimidazole-4,6-disulphonic acid, 3-methylbutyl 4-(dimethylamino)benzoate, 3,3,5-trimethylcyclohexyl 2-hydroxybenzoate (Homosalate), ethyl 4-aminobenzoate, 4,4-((6-(((1,1-dimethylethyl)amino)carbonyl)phenyl)amino) 1,3,5-triazine-2,4-diyl)diimino)bis-,bis-(2-ethyl-hexyl)ester) benzoic acid, 2,4-dihydroxy-benzophenone (Benzophenone-1), 2,2′-dihydroxy-4-methoxy-benzophenone (dioxybenzone, Benzophenone-8), 2,2′-dihydroxy-4,4′-dimethoxy-benzophenone, 2,2′,4,4′-tetrahydroxy-benzophenone, 2-hydroxy-4-methoxy-4′-methyl-benzophenone, hydroxymethoxy-benzophenone sulfonic acid, (2-Hydroxy-4-methoxyphenyl)-phenylmethanone (Benzophenone-3), 2-hydroxy-4-methoxy-benzophenone-5-sulfonic acid (Benzophenone-4) and its salts, 4-phenyl-benzophenone, 2-ethylhexyl-4′-phenyl-benzophenone-2-carboxylate, 2-hydroxy-4-n-octoxy-benzophenone, 4-hydroxy-3-carboxy-benzophenone, 2-hydroxy-4-methoxybenzophenone, 4-(2-β-glucopyranosiloxy)-propoxy-2-hydroxybenzophenone, dihydroxy-dimethoxy-disulfobenzophenone, 2-benzothiazol-2-ylphenol, [2,4′-dihydroxy-3-(2H-benzotriazol-2-yl)-5-(1,1,3,3-tetramethylbutyl)-2′-n-octoxy-5′-benzoyl]-diphenylmethane, 2,2′-hydroxy-5-methylphenyl-benzotriazole, 2-(2′-hydroxy-5′-t-octylphenyl)benzotriazole, 2-(2′-hydroxy-5′-methylphenyl)benzotriazole, 2,2′-methylene-bis-6-(2H-benzotriazol-2-yl)-4-(tetramethyl-butyl)-1,1,3,3-phenol, 2-benzoxazole-2-yl-4-methylphenol, 2-phenyl-5-methylbenzoxazole, 1,4-phenylenebis(2-benzoxazolyl), 1,3-phenylenebis(2-benzoxazolyl), 1,2-phenylenebis(2-benzoxazolyl), 2-(2-benzofuryl)-benzoxazole, 2-(benzofuryl)-5-methylbenzoxazole, 2-(3-methyl-2-benzofuryl)benzoxazole, 3-(4′-methylbenzylidene)-D,L-camphor, terephthalylidene dicamphor sulphonic acid, 3-benzylidene-D,L-camphor, 4-benzylidene-D,L-camphor, di-tert-butyl hydroxybenzylidene camphor, benzylidene camphor sulphonic acid, camphor benzalkonium methosulfate, polyacrylamido-methyl benzylidene camphor, ethylhexyl methoxycinnamate, diethanolamine p-methoxy cinnamate, isopropyl-p-acetamido cinnamate, octyl cinnamate, ethyl-4-isopropylcinnamate, methyl-2,5-diisopropyl cinnamate, ethyl-2,4-diisopropylcinnamate, methyl-2,4-diisopropylcinnamate, propyl-p-methoxycinnamate, isopropyl-p-methoxycinnamate, diisopropylcinnamate esters, isoamyl-p-methoxycinnamate, cyclohexyl-p-methoxycinnamate, ethyl-α-cyano-β-phenylcinnamate, 2-ethylhexyl-α-cyano-β-phenylcinnamate, glycerin-mono-2-ethyl-hexanoyl-diparamethoxycinnamate, glyceryl octanoate-di-p-methoxycinnamate, DEA-methoxycinnamate, methyl-bis(trimethyl-siloxy)silyl-isopentyl-trimethoxycinnamate, ethyl-α-cyano-3,5-dimethoxy-4-hydroxy cinnamate, ethyl-α-acetyl-3,5-dimethoxy-4-hydroxy cinnamate, iso-propyl-α-acetyl-3,5-dimethoxy-4-hydroxy cinnamate, iso-amyl-α-acetyl-3,5-dimethoxy-4-hydroxy cinnamate, 2-ethylhexyl-α-acetyl-3,5-dimethoxy-4-hydroxy cinnamate, ethyl-α-cyano-3-methoxy-4-hydroxy cinnamate, ethyl-α-acetyl-3-methoxy-4-hydroxy cinnamate, iso-propyl-α-acetyl-3-methoxy-4-hydroxy cinnamate, iso-amyl-α-acetyl-3-methoxy-4-hydroxy cinnamate, 2-ethylhexyl-α-acetyl-3-methoxy-4-hydroxy cinnamate, 7-ethylamino-4-methylcoumarin, 7,8-dihydroxycoumarin, 6,7-dihydroxycoumarin, 7-hydroxycoumarin, 4-methyl-7-hydroxycoumarin, diacetycurcumin, tetrahydrocurcumin diacetate, dianisoylmethane, dibenzalazine, butyl methoxydibenzoylmethane (Avobenzone), 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, ferulic acid, dimethicodiethylbenzalmalonate, diethyl-3,5-dimethoxy-4-hydroxy benzylidene malonate, di-(2-ethylhexyl)-3,5-dimethoxy-4-hydroxy benzylidene malonate, diisoamyl-3,5-dimethoxy-4-hydroxy benzylidene malonate, didodecyl-3,5-dimethoxy-4 hydroxy benzylidene malonate, dipalmitoyl-3,5-dimethoxy-4-hydroxy benzylidene malonate, di-isopropyl-3,5-dimethoxy-4-hydroxy benzylidene malonate, diethyl-3-methoxy-4-hydroxy benzylidene malonate, di-(2-ethylhexyl)-3-methoxy-4-hydroxy benzylidene malonate, diisoamyl-3-methoxy-4-hydroxy benzylidene malonate, didodecyl-3-methoxy-4-hydroxy benzylidene malonate, dipalmitoyl-3-methoxy-4-hydroxy benzylidene malonate, di-isopropyl-3-methoxy-4-hydroxy benzylidene malonate, hydrolyzed aspergillus sp. extract ferment, cerium oxide, iron oxide, manganese oxide, titanium dioxide, zinc oxide, zinc cerium oxide, zirconium oxide, 4-nitrobenzamide, 4-nitrobenzoic acid, p-aminobenzoic acid (PABA), PABA-monoglycerinester, N,N-dipropoxy-PABA-ethylester, N,N-diethoxy PABA-ethylester, N,N-dimethyl PABA-ethylester, N,N-dimethyl PABA-butylester, PEG-25 PABA, N,N-dimethyl PABA-amylester, N,N-dimethyl PABA-octlylester (2-ethylhexyl 4-(dimethylamino)benzoate, Padimate O), octyl dimethyl PABA, ethyl-dihydroxypropyl-PABA, isoamyl dimethyl PABA (3-methylbutyl 4-(dimethylamino)benzoate, Padimate A), 1-(3,4-dimethoxyphenyl)-4,4-dimethyl-1,3-pentanedione, 5-(3,3-Dimethyl-2-norbornyliden)-3-pentane-2-one, 1-(4-methoxyphenyl)-3-(4-tert-butylphenyl)propane-1,3-dione, dipropylene glycol salicylate, 2-ethylhexyl 2-cyano-3,3-diphenyl-2-propenoate (Octocrylene), hydrolyzed soy extract, hydrolyzed linseed extract, sunflower seed extract, grape seed extract, pinus pinaster bark extract, pinus pinaster bud extract, ginseng extract ferment, spirulina platensis powder, phenyl salicylate, p-tert-butylphenyl salicylate, p-octylphenyl salicylate, 2-ethylhexyl salicylate (octyl salicylate), amyl salicylate, menthyl salicylate, homomethyl salicylate, benzyl salicylate, p-isopropanol-phenyl salicylate, TEA-salicylate, 2-(2H-benzotriazol-2-yl)-4-methyl-6-[2-methyl-3-[1,3,3,3-tetramethyl-1-[(trimethylsilyl)oxy]disiloxanyl]propyl]-phenol (Mexoryl XL), 2-[p-(tert-butylamido)anilino]-4,6-bis[p-(2′-ethylhexyl-1′-oxycarbonyl)anilino]-1,3,5-triazine (INCI name: Diethylhexyl Butamido Triazone), 2,4,6-tris[4-(2-ethylhexyl-oxycarbonyl)-anilino]-1,3,5-triazine (INCI name: Ethylhexyl Triazone), (1,3,5)-triazine-2,4-bis((4-2-ethyl-hexyloxy)-2-hydroxy)-phenyl)-6-(4-methoxy-phenyl) (bemotrizinol), octyl triazone (CAS-no.: 88122-99-0), 2,4-bis(2,4-dihydroxyphenyl)-6-(1-methyl-1H-pyrazol-5-yl)-1,3,5-triazine, 2,4-bis{[4-(2-ethylhexyloxy)-2-hydroxy]phenyl}-6-(1-methyl-1H-pyrazol-5-yl)-1,3,5-triazine, 2-[2,4-bis(2-ethylhexyloxy)phenyl]-4-[4-(2-ethylhexyloxy)-2-hydroxyphenyl]-6-(1-methyl-1H-pyrazol-5-yl)-1,3,5-triazine, 2,4-bis(2,4-dihydroxyphenyl)-6(1-phenyl-1H-pyrazol-5-yl)-1,3,5-triazine, 2,4-bis{[4-(2-ethylhexyloxy)-2-hydroxy]phenyl}-6-(1-phenyl-1H-pyrazol-5-yl)-1,3,5-triazine, 2,4-bis [4-(butoxycarbonyl)phenylamino]-6-(1-methyl-1H-pyrazol-5-yl)-1,3,5-triazine, 2,4-bis{4-[(2-(ethylhexyloxy)carbonyl]phenylamino}-6-(1-methyl-1H-pyrazol-5-yl)-1,3,5-triazine, 2,4-bis[4-(butoxycarbonyl)phenylamino]-6-(1-phenyl-1H-pyrazol-5-yl)-1,3,5-triazine, 2,4-bis{4-[(2-ethylhexyloxy)carbonyl]phenylamino}-6-(1-phenyl-1H-pyrazol-5-yl)-1,3,5-triazine and 2-[(4-butoxycarbonyl)phenylamino]-4,6-bis(1-methyl-1H-pyrazol-5-yl)-1,3,5-triazine, 2-(1-benzyl-1H-pyrrole-2-yl)-4,6-bis(2,4-dihydroxyphenyl)-1,3,5-triazine, 2-(1-benzyl-1H-pyrrole-2-yl)-4,6-bis[4-(2-ethylhexyloxy)-2-hydroxyphenyl]-1,3,5-triazine, 2-(1-benzyl-1H-pyrrole-2-yl)-4,6-bis[4-(butoxycarbonyl)phenylamino]-1,3,5-triazine, 2-(1-benzyl-1H-pyrrole-2-yl)-4,6-bis(biphenyl-4-ylamino)-1,3,5-triazine, 2-(1-benzyl-1H-pyrrole-2-yl)-4,6-bis(4-benzoylphenylamino)-1,3,5-triazine, 2-(1-benzyl-1H-pyrrole-2-yl)-4,6-bis(9-oxo-9H-fluoren-3-ylamino)-1,3,5-triazine, 2-(1-benzyl-1H-pyrrole-2-yl)-4,6-bis[4-(imidazo[1,2-a]pyridin-2-yl)phenylamino]-1,3,5-triazine, 2-(1-benzhydryl-1H-pyrrole-2-yl)-4,6-bis[4-(butoxycarbonyl)phenylamino]-1,3,5-triazine, 2-(1-benzyl-1H-pyrrole-2-yl)-4,6-bis[4-(6,6-dimethyl-4-oxo-4,5,6,7-tetrahydroindol-1-yl)phenylamino]-1,3,5-triazine, 2-(1-benzyl-1H-pyrrole-2-yl)-4,6-bis[4-((2-ethylhexyloxy)carbonyl)phenylamino]-1,3,5-triazine, 2-(1-benzyl-1H-pyrrole-2-yl)-4,6-bis[4-1H-imidazo[1,2-a]pyridin-2-yl)phenylamino]-1,3,5-triazine, 2-(1-benzyl-1H-pyrrole-2-yl)-4,6-bis[4-(1H-benzo[d]imidazol-2-yl)phenylamino]-1,3,5-triazine, 2-(1-benzyl-1H-pyrrole-2-yl)-4,6-bis[4-(1H-pyrazol-1-yl)phenylamino]-1,3,5-triazine, 2-(1-benzyl-1H-pyrrole-2-yl)-4,6-bis[naphthalen-2-ylamino]-1,3,5-triazine, 2-(1-benzyl-1H-pyrrole-2-yl)-4,6-bis[4-((E)-3-ethoxy-3-oxoprop-1-enyl)phenylamino]-1,3,5-triazine, 2-(1-benzyl-1H-pyrrole-2-yl)-4,6-bis[4-(E)-styrylphenylamino]-1,3,5-triazine, urocanic acid, ethyl-urocanic acid, talc, kaolin, calcium carbonate, bisoctrizole (CAS-no.: 103597-45-I), bornelone (CAS-no.: 2226-11-1), 2-Hydroxy-1,4-naphthochinone (Lawsone, CAS-no.: 83-72-7), petrolatum red, phenol sulphonate, sodium 3,4-dimethylphenyl-glyoxylate, 3,3′-(1,4-phenylenedimethylene-bis-(7,7-dimethyl-2-oxobicyclo-[2,2,1]hept-1-yl-methanesulfonic acid, α-(2-oxoborn-3-ylidene)-toluene-4-sulphonic acid, 2-(2H-benzotriazole-2-yl)-4-methyl-6-[2-methyl-3-[1,3,3,3-tetramethyl-1-[(trimethylsilyl)oxy]disiloxanyl]propyl]-phenol (INCI name: drometrizole trisiloxane, DTS), digalloyl trioleate, dimethicone/PEG-15 crosspolymer, ethylhexyl-bis-isopentyl-benzoxazolyl-phenyl melamine and 2-ethylhexyl-dimethoxy-benzylidene-dioxo-imidazolidine propionate;

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds;
wherein the UV-absorbing active substance (B) has its absorption maximum λ_{max(B)} in the range of 290 to 400 nm, preferably with the proviso that λ_{max(B)} of active substance (B) does not lie in the same UV radiation range wherein the λ_{max(A)} of active substance (A) lies.

In a further preferred embodiment of the present invention the active substance (B) is at least one compound of general formula (X)

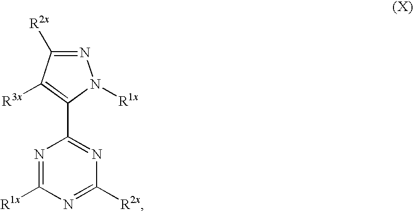

wherein

R^{1x} represents a hydrogen atom; an optionally substituted cycloalkyl radical from 3 to 7 carbon atoms; an aryl radical which is unsubstituted or at least mono-substituted with a group selected from aryl, halogen, an —O—C₁₋₆ radical and a C₁₋₆ alkyl radical; a -phenyl-C₁₋₆ alkyl radical; a linear or branched C₁₋₈ alkyl radical which is unsubstituted or at least mono-substituted with a moiety selected from —SO₃M^{x}, —N(R^{4x})₃⁺ and a radical of general formula (XX)

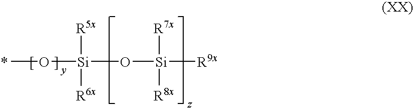

wherein
y=0 or 1;
z=0, 1, 2, 3 or 4;

R^{5x}, R^{6x}, R^{7x}, R^{8x} and R^{9x}, independent of each other, each represents an unsubstituted or at least mono-substituted C₁₋₆ alkyl radical; an —O—C₁₋₆ radical; an unsubstituted or at least mono-substituted aryl radical and an —OSi(R^{10x})₃ radical;

R^{10x} represents a C₁₋₆ alkyl radical; an —O—C₁₋₆ radical; or an unsubstituted or at least mono-substituted aryl radical;

M^{x} represents a hydrogen atom, Na⁺ or K⁺;

R^{4x} represents an unsubstituted or at least mono-substituted C₁₋₆ alkyl radical;

R^{2x} and R^{3x}, independent of each other, each represents a hydrogen atom; an unsubstituted or at least monosubstituted C₁₋₄ alkyl radical; or an unsubstituted or at least mono-substituted aryl radical;

A^{1x} represents a radical of general formula (X1) or (X2)

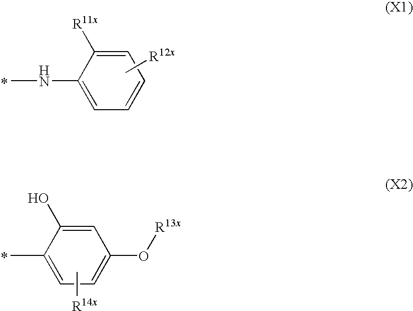

A^{2x} represents a radical of general formula (X1), (X3) or (X4)

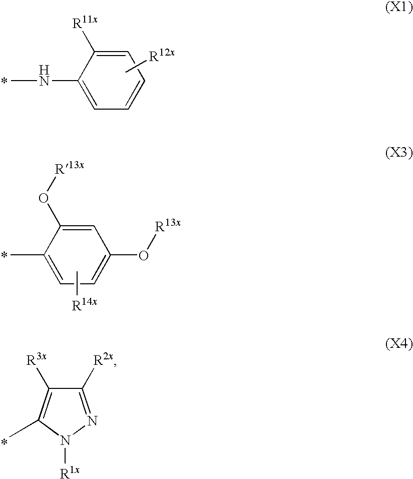

wherein

R^{11x} represents a hydrogen atom; a linear or branched, saturated or unsaturated, unsubstituted or at least monosubstituted C₁₋₆ aliphatic radical; or a hydroxy (—OH) residue;

R^{12x} represents a hydrogen atom; a —C(═O)—OR^{15x} radical; a —C(═O)—NR^{16x}R^{17x} radical; an unsubstituted or at least monosubstituted —O—C₁₋₁₈ radical; an unsubstituted or at least monosubstituted —O-phenyl radical; a C₅₋₇ cycloalkyl radical; a phenyl or a naphtyl radical; a phenyl or a naphtyl radical which are substituted by 1 or 2 substituents which are independently from one another selected from the group consisting of phenyl, Cl, —O—C₁₋₆ alkyl and C₁₋₆ alkyl; a linear or branched, saturated or unsaturated C₁₋₁₈ aliphatic radical which is unsubstituted or at least mono-substituted with —OH, -phenyl or un moiety of general formula (XX) as defined above;

R^{15x}, R^{16x} and R^{17x}, independent of each other, each represents a hydrogen atom; a linear or branched, unsubstituted or at least mono-substituted C₁₋₁₈ alkyl radical;

R^{14x} represents hydrogen atom; or —SO₃M^{x}, with M^{x} being H, Na⁺ or K⁺;

R^{13x} and R^{3x}, independent of each other, each represents a hydrogen atom; an unsubstituted or at least monosubstituted C(═O)—C₁₋₁₈ radical; a linear or branched C₁₋₁₈ alkyl radical, which is unsubstituted or at least mono-substituted with at least one hydroxy (—OH) residue, a SO₃M^{x} moiety, a —N(R^{4x})₃⁺ moiety, wherein M^{x} and R^{4x} have the meaning as defined above, or a radical of general formula (XX) as defined above;

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds of general formula (X);
and/or
at least one compound of general formula (Is)

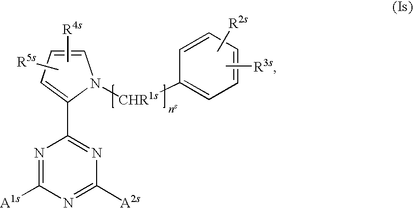

wherein
n^{s}=0, 1, 2, 3 or 4;

R^{1s} represents a hydrogen atom; a linear or branched, unsubstituted or at least monosubstituted C₁₋₃ alkyl radical; or a R^{2s′}R^{3s′-}substituted phenyl-radical;

R^{2s}, R^{2s′}, R^{3s} and R^{3s′}, independent of each other, each represents a hydrogen atom; a halogen atom; a hydroxy (—OH) residue; an unsubstituted or at least monosubstituted C₁₋₃ alkyl radical; an unsubstituted or at least monosubstituted —O—C₁₋₃ radical; or an unsubstituted or at least monosubstituted aryl radical;

preferably with the condition that if n=0, R^{2s} or R^{3s} does not represent an acryl radical;
or

R^{2s} and R^{3s} form together with the phenyl ring to which they are attached, an unsubstituted or at least mono-substituted naphthalene ring;

R^{4s} and R^{5s}, independent of each other, each represents a hydrogen atom; an unsubstituted or at least monosubstituted C₁₋₄ alkyl radical; or an unsubstituted or at least mono-substituted aryl radical;

A^{1s} represents a radical of general formula (IIs), (IIIs) or (IVs)

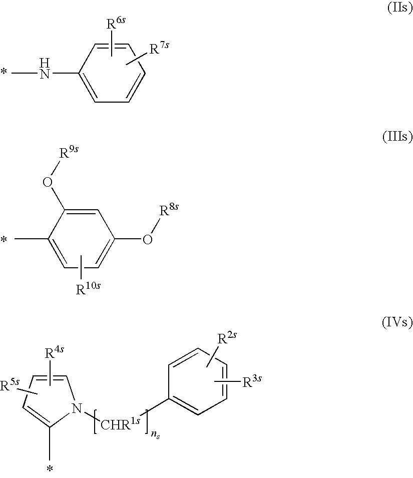

A^{2s} represents a radical of general formula (IIs) or (Vs)

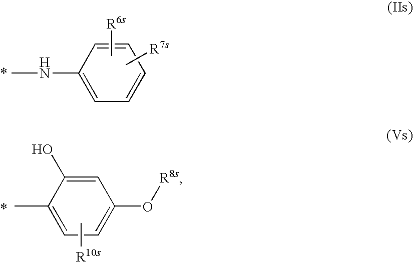

wherein

R^{6s} represents a hydrogen atom; a linear or branched, unsubstituted or at least monosubstituted C₁₋₆ alkyl radical; or a hydroxy (—OH) residue;

R^{7s} represents a hydrogen atom; an unsubstituted or at least monosubstituted, saturated or unsaturated, C₃₋₆ cycloalkyl radical; an unsubstituted or at least monosubstituted, saturated or unsaturated C₁₋₁₀ cycloalkyl radical having from 5 to 10 atoms that can contain 1, 2 or 3 heteroatom(s) as ring member(s) independently selected from the group consisting of O, N and S; an unsubstituted or at least monosubstituted aryl radical; an unsubstituted or at least monosubstituted heteroaryl radical having from 5 to 10 atoms that can contain 1, 2 or 3 heteroatom(s) as ring member(s) independently selected from the group consisting of O, N and S; a —C(═O)—OR^{11s} radical; a —C(═O)—NR^{12s}R^{13s} radical; an unsubstituted or at least monosubstituted —O—C₁₋₁₈ radical; an unsubstituted or at least monosubstituted —O-phenyl or —O-naphtyl radical; an unsubstituted or at least monosubstituted —COR^{14s} radical; a linear or branched, saturated or unsaturated, C₁₋₁₈ aliphatic radical, which is unsubstituted or at least mono-substituted with at least one hydroxy (—OH) residue, a SO₃M^{s} moiety, a —N(R^{15s})₃⁺ moiety or a radical of general formula (VIs)

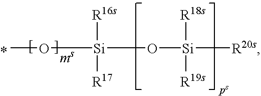

wherein
m^{s}=0 or 1;
p^{s}=0, 1, 2, 3 or 4;

R^{16s}, R^{17s}, R^{18s}, R^{19s} and R^{20s}, independent of each other, each represents an unsubstituted or at least mono-substituted C₁₋₆ alkyl radical; an unsubstituted or at least mono-substituted —O—C₁₋₆ radical; an unsubstituted or at least mono-substituted aryl radical; or an —O—Si(R^{21s})₃ radical;

R^{21s} represents an unsubstituted or at least mono-substituted C₁₋₆ alkyl radical; an —O—C₁₋₆ radical; an unsubstituted or at least mono-substituted aryl radical;

R^{11s}, R^{12s} and R^{13s}, independent of each other, each represents a hydrogen atom; a linear or branched, unsubstituted or at least monosubstituted C₁₋₁₈ alkyl radical; an unsubstituted or at least mono-substituted C₃₋₆ cycloalkyl radical;

or

R^{12s} and R^{13s} form together with the bridging nitrogen atom an unsubstituted or at least mono-substituted, saturated 5 to 7-membered cycloaliphatic radical, which contains 1, 2 or 3 heteroatom(s) as ring member(s) independently selected from the group consisting of O, N and S;

R^{14s} represents an unsubstituted or at least mono-substituted C₁₋₁₈ alkyl radical; or an unsubstituted or at least mono-substituted aryl radical;

R^{15s} represents an unsubstituted or at least mono-substituted C₁₋₁₈ alkyl radical;

M^{s} represents H, Na⁺ or K⁺;

R^{6s} and R^{7s} form together with the phenyl ring an unsubstituted or at least mono-substituted 9 to 15-membered polycyclic ring system;

or

R^{6s} and R^{14s} form together with the phenyl ring an unsubstituted or at least mono-substituted 9 to 15-membered polycyclic ring system;

R^{8s} and R^{9s}, independent of each other, each represents a hydrogen atom; an unsubstituted or at least monosubstituted —C(═O)—C₁₋₁₈ radical; a linear or branched, saturated or unsaturated C₁₋₁₈ aliphatic radical, which is unsubstituted or at least mono-substituted with at least one hydroxy (—OH) residue, a —SO₃M^{s} moiety, a —N(R^{15s})₃⁺ moiety or a radical of general formula (VIs) as defined above;

R^{10s} represents a hydrogen atom; or a —SO₃M^{s} moiety with M^{s} representing H, Na⁺ or K⁺;

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds of general formula (Is);
wherein the UV-absorbing active substance (B) has its absorption maximum λ_{max(B)} in the range of 290 to 400 nm, preferably with the proviso that λ_{max(B)} of active substance (B) does not lie in the same UV radiation range wherein the λ_{max(A)} of active substance (A) lies.

The compounds of general formulae (X) and (Is) can be prepared according to WO 2006/064366 and WO 2006/128920, respectively, which relevant disclosure is herewith introduced by reference and shall form part of the description.

According to a further preferred embodiment of the present invention the UV-absorbing active substance (A) is at least one heptaazaphenalene compound selected from the group comprising
2,5,8-tris-(4-(butoxycarbonyl)phenylamino)-1,3,4,6,7,9,9b-heptaazaphenalene,
2,5,8-tris-(4-(2-ethylhexyloxycarbonyl)phenylamino)-1,3,4,6,7,9,9b-heptaazaphenalene,
2,5,8-tris-(4-(imidazo[1,2-a]pyridin-2-yl)-phenylamino)-1,3,4,6,7,9,9b-heptaazaphenalene,
2,5,8-tris-(4-(4,5,6,7-tetrahydro-2,6,6-trimethyl-4-oxo indol-1-yl)phenylamine)-1,3,4,6,7,9,9b-heptaazaphenalene,
2,5,8-tris-(biphenyl-4-ylamino)-1,3,4,6,7,9,9b-heptaazaphenalene,
2,5,8-tris-(4-(1H-benzo[d]imidazol-2-yl)phenylamino)-1,3,4,6,7,9,9b-heptaazaphenalene,
2,5-bis-(biphenyl-4-ylamino)-8-(4-(butoxycarbonyl)phenylamino)-1,3,4,6,7,9,9b-heptaazaphenalene,
2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene,
2,5,8-tris-(2,4-dihydroxyphenyl)-1,3,4,6,7,9,9b-heptaazaphenalene,
2,5-dichloro-8-(1-methyl-1H-pyrrole-2-yl)-1,3,4,6,7,9,9b-heptaazaphenalene,
2,5-bis-(4-(butoxycarbonyl)phenylamino)-8-(2-(1-methyl-1H-pyrrole-2-yl)-1,3,4,6,7,9,9b-heptaazaphenalene,
2-(4-(carboxy)phenylamino)-5,8-bis-(4-methylphenyl)-1,3,4,6,7,9,9b-heptaazaphenalene,
2,5,8-tris-(4-(4,5,6,7-tetrahydro-2,6,6-trimethyl-4-oxo indol-1-yl)phenylamine)-1,3,4,6,7,9,9b-heptaazaphenalene,
2,5-bis-(4-(butoxycarbonyl)phenylamino)-8-(1-methyl-1H-pyrrole-2-yl)-1,3,4,6,7,9,9b-heptaazaphenalene,
2,5-bis(2,4-dihydroxyphenyl)-8-(1-methyl-1H-pyrazol-5-yl)-1,3,4,6,7,9,9b-heptaazaphenalene,
2,5-bis{[4-(2-ethylhexyloxy)-2-hydroxy]phenyl}-6-(1-methyl-1H-pyrazol-5-yl)-1,3,4,6,7,9,9b-heptaazaphenalene,
2-[2,4-bis(2-ethylhexyloxy)phenyl]-5-[4-(2-ethylhexyl oxy)-2-hydroxyphenyl]-8-(1-methyl-1H-pyrazol-5-yl)-1,3,4,6,7,9,9b-heptaazaphenalene,
2,5-bis(2,4-dihydroxyphenyl)-8-(1-phenyl-1H-pyrazol-5-yl)-1,3,4,6,7,9,9b-heptaazaphenalene,
2,5-bis{[4-(2-ethylhexyloxy)-2-hydroxy]phenyl}-8-(1-phenyl-1H-pyrazol-5-yl)-1,3,4,6,7,9,9b-heptaazaphenalene,
2,5-bis[4-(butoxycarbonyl)phenylamino]-8-(1-methyl-1H-pyrazol-5-yl)-1,3,4,6,7,9,9b-heptaazaphenalene,
2,5-bis{4-[(2-(ethylhexyloxy)carbonyl]phenylamino}-8-(1-methyl-1H-pyrazol-5-yl)-1,3,4,6,7,9,9b-heptaazaphenalene,
2,5-bis[4-(butoxycarbonyl)phenylamino]-8-(1-phenyl-1H-pyrazol-5-yl)-1,3,4,6,7,9,9b-heptaazaphenalene,
2,5-bis{4-[(2-ethylhexyloxy)carbonyl]phenylamino}-8-(1-phenyl-1H-pyrazol-5-yl)-1,3,4,6,7,9,9b-heptaazaphenalene,
2-(1-benzyl-1H-pyrrole-2-yl)-5,8-bis(2,4-dihydroxyphenyl)-1,3,4,6,7,9,9b-heptaazaphenalene,
2-(1-benzyl-1H-pyrrole-2-yl)-5,8-bis[4-(2-ethylhexyloxy)-2-hydroxyphenyl]-1,3,4,6,7,9,9b-heptaazaphenalene,
2-(1-benzyl-1H-pyrrole-2-yl)-5,8-bis[4-(butoxycarbonyl)phenylamino]-1,3,4,6,7,9,9b-heptaazaphenalene,
2-(1-benzyl-1H-pyrrole-2-yl)-5,8-bis(biphenyl-4-ylamino)-1,3,4,6,7,9,9b-heptaazaphenalene,
2-(1-benzyl-1H-pyrrole-2-yl)-5,8-bis(4-benzoylphenylamino)-1,3,4,6,7,9,9b-heptaazaphenalene,
2-(1-benzyl-1H-pyrrole-2-yl)-5,8-bis(9-oxo-9H-fluoren-3-ylamino)-1,3,4,6,7,9,9b-heptaazaphenalene,
2-(4-(tert-butylcarbamoyl)phenylamino)-5,8-bis-(4-(2-ethylhexyloxycarbonyl)phenylamino)-1,3,4,6,7,9,9b-heptaazaphenalene,
2,5-bis-[(4-(2-ethylhexyloxy)-2-hydroxy)-phenyl]-8-(4-methoxyphenyl)-1,3,4,6,7,9,9b-heptaazaphenalene,
2-(2-ethylhexylamino)-5,8-bis-(4-(5-(1,1-dimethylpropyl)benzo[d]oxazol-2-yl)phenylamino)-1,3,4,6,7,9,9b-heptaazaphenalene,
2,5,8-tris-(4-(1H-pyrazol-1-yl)phenylamino)-1,3,4,6,7,9,9b-heptaazaphenalene
2,5,8-tris-(4-benzoylphenylamino)-1,3,4,6,7,9,9b-heptaazaphenalene,
2,5,8-tris-(4-butoxy-2-hydroxyphenyl)-1,3,4,6,7,9,9b-heptaazaphenalene,
2,5,8-tris-(naphthalen-2-ylamino)-1,3,4,6,7,9,9b-heptaazaphenalene,
2,5-bis-(4-(butoxycarbonyl)phenylamino)-8-(2-(1-methyl-1H-indol-3-yl)-1,3,4,6,7,9,9b-heptaazaphenalene,
2,5,8-tris-(biphenyl-4-yloxy)-1,3,4,6,7,9,9b-heptaazaphenalene,
2,5,8-tris-(3-methoxyphenylamino)-1,3,4,6,7,9,9b-heptaazaphenalene,
2,5,8-tris-(4-methoxyphenylamino)-1,3,4,6,7,9,9b-heptaazaphenalene,
2,5,8-tris-(4-((E)-3-ethoxy-3-oxoprop-1-enyl)phenylamino)-1,3,4,6,7,9,9b-heptaazaphenalene,
2,5,8-tris-(methoxycarbonyl-4′-biphenyl-4-ylamino)-1,3,4,6,7,9,9b-heptaazaphenalene,
2,5,8-tris-(4-(methoxycarbonyl)phenylamino)-1,3,4,6,7,9,9b-heptaazaphenalene,
2,5,8-tris-(4-(1H-benzo[d]imidazol-2-yl)-3-hydroxyphenylamino)-1,3,4,6,7,9,9b-heptaazaphenalene,
2,5,8-tris-(4-(phenylamino)phenylamino)-1,3,4,6,7,9,9b-heptaazaphenalene,
2,5,8-tris-((4-(E)-styrylphenyl)amino)-1,3,4,6,7,9,9b-heptaazaphenalene,
2,5,8-tris-(2-ethylhexylamino)-1,3,4,6,7,9,9b-heptaazaphenalene,
2,5,8-tris-(4-(L-menthylcarbonyl)phenylamino)-1,3,4,6,7,9,9b-heptaazaphenalene,
2,5,8-tris-(4-((3,3,5-trimethylcyclohexyloxy)carbonyl)phenylamino)-1,3,4,6,7,9,9b-heptaazaphenalene,
2,5,8-tris-(4-((E)-3-(2-ethylhexyloxy)-3-oxoprop-1-enyl)phenylamino)-1,3,4,6,7,9,9b-heptaazaphenalene,
2-(3-((E)-3-(2-ethylhexyloxy)-3-oxoprop-1-enyl)phenylamino)-5,8-bis-(4-((E)-3-(2-ethylhexyloxy)-3-oxoprop-1-enyl)phenylamino)-1,3,4,6,7,9,9b-heptaazaphenalene,
2,5,8-tris-(3-nitrophenylamino)-1,3,4,6,7,9,9b-heptaazaphenalene,
2,5,8-tris-(4-(2-butyloctyloxycarbonyl)phenylamino)-1,3,4,6,7,9,9b-heptaazaphenalene,
2,5,8-tris-(4-(2-hexyldecyloxycarbonyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(hexyldecyloxycarbonyl)phenylamino)-1,3,4,6,7,9,9b-heptaazaphenalene,
2,5,8-tris-(4-(octyldecyloxycarbonyl)phenylamino)-1,3,4,6,7,9,9b-heptaazaphenalene,
2,5,8-tris-((3-(E)-styrylphenyl)amino)-1,3,4,6,7,9,9b-heptaazaphenalene,
2,5,8-tris-(4-((3,5,5-trimethylhexyloxy)carbonylphenylamino)-1,3,4,6,7,9,9b-heptaazaphenalene,
2,5-bis-(3-(methoxy)phenylamino)-8-(2-(1-methyl-1H-pyrrole-2-yl)-1,3,4,6,7,9,9b-heptaazaphenalene,
2,5,8-tris-(4-((2-ethylhexyl)carbamoyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(1,3,3-trimethylbicyclo[2.2.1]heptan-2-yloxy)phenylamino)-1,3,4,6,7,9,9b-heptaazaphenalene,
2,5,8-tris-(1H-indol-5-ylamino)-1,3,4,6,7,9,9b-heptaazaphenalene,
2,5,8-tris-(4-((3,7-dimethyloctyloxy)carbonyl)phenylamino)-1,3,4,6,7,9,9b-heptaazaphenalene and
2,5,8-tris-(2-amino-4,5-dimethylphenylamino)-1,3,4,6,7,9,9b-heptaazaphenalene;
   and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof;
   which active substance (A) has its absorption maximum λ_{max(A)} in the range of either UV-A or UV-B radiation
   and the UV-absorbing active substance (B)
   is at least one inorganic compound selected from the group comprising cerium oxide, iron oxide, manganese oxide, titanium dioxide, zinc oxide, zinc cerium oxide and zirconium oxide;
   and/or
   is at least one UV-absorbing organic compound selected from the group consisting of 2-ethylhexyl 2-cyano-3,3′-diphenylacrylate, ethyl 2-cyano-3,3′-diphenylacrylate, polymer of N-{(2 and 4)[(2-oxoborn-3-ylidene)methyl]benzyl}acrylamide, methyl 4-aminobenzoate, butyl 4-aminobenzoate, 5-methyl-2-(1-methylethyl)-cyclohexyl 2-aminobenzoate, glyceryl aminobenzoate, 2-ethylhexyl-p-dimethylethyl-aminobenzoate, 4-aminobenzamide, homomethyl-N-acetyl-anthranilic acid, menthyl antrhanilate, 2-(1H-benzimidazol-2-yl)-4-methoxyphenol, 2,2′-bis(benzimidazole), monosodium salt of 2-2′-bis-(1,4-phenylene)1H-benzimidazole-4,6-disulphonic acid, 2-phenylbenzimidazole-5-sulphonic acid, 5,5′,6,6′-tetramethyl-2,2′-bis(benzimidazole), 5,5′-dimethyl-2,2′-bis(benzimidazole), 6-methoxy-2,2′-bis(benzimidazole), 1,4-phenylenebis(2-benzimidazolyl), 1,2-phenylenebis)benzimidazolyl), 1,4-phenylenebis(N-2-ethylhexyl-2-benzimidazolyl), 1,4-phenylenebis(N-tri-methylsilylmethyl-2-benzimidazolyl), 2-(1H-benzimidazol-2-yl)benzothiazole, 2-(1H-benzimidazol-2-yl)benzoxazole, N,N′-dimethyl-2,2′-bis(benzimidazole), phenylbenzimidazole-5-sulfonic acid (Ensulizole) and its salts, 2-2′-bis-(1,4-phenylene)1H-benzimidazole-4,6-disulphonic acid, 3-methylbutyl 4-(dimethylamino)benzoate, 3,3,5-trimethylcyclohexyl 2-hydroxybenzoate (Homosalate), ethyl 4-aminobenzoate, 4,4-((6-(((1,1-dimethylethyl)amino)carbonyl)phenyl)amino) 1,3,5-triazine-2,4-diyl)diimino)bis-,bis-(2-ethyl-hexyl)ester) benzoic acid, 2,4-dihydroxy-benzophenone (Benzophenone-1), 2,2′-dihydroxy-4-methoxy-benzophenone (dioxybenzone, Benzophenone-8), 2,2′-dihydroxy-4,4′-dimethoxy-benzophenone, 2,2′,4,4′-tetrahydroxy-benzophenone, 2-hydroxy-4-methoxy-4′-methyl-benzophenone, hydroxymethoxy-benzophenone sulfonic acid, (2-Hydroxy-4-methoxyphenyl)-phenylmethanone (Benzophenone-3), 2-hydroxy-4-methoxy-benzophenone-5-sulfonic acid (Benzophenone-4) and its salts, 4-phenyl-benzophenone, 2-ethylhexyl-4′-phenyl-benzophenone-2-carboxylate, 2-hydroxy-4-n-octoxy-benzophenone, 4-hydroxy-3-carboxy-benzophenone, 2-hydroxy-4-methoxybenzophenone, 4-(2-β-glucopyranosiloxy)-propoxy-2-hydroxybenzophenone, dihydroxy-dimethoxy-disulfobenzophenone, 2-benzothiazol-2-ylphenol, [2,4′-dihydroxy-3-(2H-benzotriazol-2-yl)-5-(1,1,3,3-tetramethylbutyl)-2′-n-octoxy-5′-benzoyl]-diphenylmethane, 2,2′-hydroxy-5-methylphenyl-benzotriazole, 2-(2′-hydroxy-5′-t-octylphenyl)benzotriazole, 2-(2′-hydroxy-5′-methylphenyl)benzotriazole, 2,2′-methylene-bis-6-(2H-benzotriazol-2-yl)-4-(tetramethyl-butyl)-1,1,3,3-phenol, 2-benzoxazole-2-yl-4-methylphenol, 2-phenyl-5-methylbenzoxazole, 1,4-phenylenebis(2-benzoxazolyl), 1,3-phenylenebis(2-benzoxazolyl), 1,2-phenylenebis(2-benzoxazolyl), 2-(2-benzofuryl)-benzoxazole, 2-(benzofuryl)-5-methylbenzoxazole, 2-(3-methyl-2-benzofuryl)benzoxazole, 3-(4′-methylbenzylidene)-D,L-camphor, terephthalylidene dicamphor sulphonic acid, 3-benzylidene-D,L-camphor, 4-benzylidene-D,L-camphor, di-tert-butyl hydroxybenzylidene camphor, benzylidene camphor sulphonic acid, camphor benzalkonium methosulfate, polyacrylamido-methyl benzylidene camphor, ethylhexyl methoxycinnamate, diethanolamine p-methoxy cinnamate, isopropyl-p-acetamido cinnamate, octyl cinnamate, ethyl-4-isopropylcinnamate, methyl-2,5-diisopropyl cinnamate, ethyl-2,4-diisopropylcinnamate, methyl-2,4-diisopropylcinnamate, propyl-p-methoxycinnamate, isopropyl-p-methoxycinnamate, diisopropylcinnamate esters, isoamyl-p-methoxycinnamate, cyclohexyl-p-methoxycinnamate, ethyl-α-cyano-β-phenylcinnamate, 2-ethylhexyl-α-cyano-β-phenylcinnamate, glycerin-mono-2-ethyl-hexanoyl-diparamethoxycinnamate, glyceryl octanoate-di-p-methoxycinnamate, DEA-methoxycinnamate, methyl-bis(trimethyl-siloxy)silyl-isopentyl-trimethoxycinnamate, ethyl-α-cyano-3,5-dimethoxy-4-hydroxy cinnamate, ethyl-α-acetyl-3,5-dimethoxy-4-hydroxy cinnamate, iso-propyl-α-acetyl-3,5-dimethoxy-4-hydroxy cinnamate, iso-amyl-α-acetyl-3,5-dimethoxy-4-hydroxy cinnamate, 2-ethylhexyl-α-acetyl-3,5-dimethoxy-4-hydroxy cinnamate, ethyl-α-cyano-3-methoxy-4-hydroxy cinnamate, ethyl-α-acetyl-3-methoxy-4-hydroxy cinnamate, iso-propyl-α-acetyl-3-methoxy-4-hydroxy cinnamate, iso-amyl-α-acetyl-3-methoxy-4-hydroxy cinnamate, 2-ethylhexyl-α-acetyl-3-methoxy-4-hydroxy cinnamate, 7-ethylamino-4-methylcoumarin, 7,8-dihydroxycoumarin, 6,7-dihydroxycoumarin, 7-hydroxycoumarin, 4-methyl-7-hydroxycoumarin, diacetycurcumin, tetrahydrocurcumin diacetate, dianisoylmethane, dibenzalazine, butyl methoxydibenzoylmethane (Avobenzone), 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, ferulic acid, dimethicodiethylbenzalmalonate, diethyl-3,5-dimethoxy-4-hydroxy benzylidene malonate, di-(2-ethylhexyl)-3,5-dimethoxy-4-hydroxy benzylidene malonate, diisoamyl-3,5-dimethoxy-4-hydroxy benzylidene malonate, didodecyl-3,5-dimethoxy-4 hydroxy benzylidene malonate, dipalmitoyl-3,5-dimethoxy-4-hydroxy benzylidene malonate, di-isopropyl-3,5-dimethoxy-4-hydroxy benzylidene malonate, diethyl-3-methoxy-4-hydroxy benzylidene malonate, di-(2-ethylhexyl)-3-methoxy-4-hydroxy benzylidene malonate, diisoamyl-3-methoxy-4-hydroxy benzylidene malonate, didodecyl-3-methoxy-4-hydroxy benzylidene malonate, dipalmitoyl-3-methoxy-4-hydroxy benzylidene malonate, di-isopropyl-3-methoxy-4-hydroxy benzylidene malonate, hydrolyzed aspergillus sp. extract ferment, 4-nitrobenzamide, 4-nitrobenzoic acid, p-aminobenzoic acid (PABA), PABA-monoglycerinester, N,N-dipropoxy-PABA-ethylester, N,N-diethoxy PABA-ethylester, N,N-dimethyl PABA-ethylester, N,N-dimethyl PABA-butylester, PEG-25 PABA, N,N-dimethyl PABA-amylester, N,N-dimethyl PABA-octlylester (2-ethylhexyl 4-(dimethylamino)benzoate, Padimate O), octyl dimethyl PABA, ethyl-dihydroxypropyl-PABA, isoamyl dimethyl PABA (3-methylbutyl 4-(dimethylamino)benzoate, Padimate A), 1-(3,4-dimethoxyphenyl)-4,4-dimethyl-1,3-pentanedione, 5-(3,3-Dimethyl-2-norbornyliden)-3-pentane-2-one, 1-(4-methoxyphenyl)-3-(4-tert-butylphenyl)propane-1,3-dione, dipropylene glycol salicylate, 2-ethylhexyl 2-cyano-3,3-diphenyl-2-propenoate (Octocrylene), hydrolyzed soy extract, hydrolyzed linseed extract, sunflower seed extract, grape seed extract, pinus pinaster bark extract, pinus pinaster bud extract, ginseng extract ferment, spirulina platensis powder, phenyl salicylate, p-tert-butylphenyl salicylate, p-octylphenyl salicylate, 2-ethylhexyl salicylate (octyl salicylate), amyl salicylate, menthyl salicylate, homomethyl salicylate, benzyl salicylate, p-isopropanol-phenyl salicylate, TEA-salicylate, 2-(2H-benzotriazol-2-yl)-4-methyl-6-[2-methyl-3-[1,3,3,3-tetramethyl-1-[(trimethylsilyl)oxy]disiloxanyl]propyl]-phenol (Mexoryl XL), 2-[p-(tert-butylamido)anilino]-4,6-bis[p-(2′-ethylhexyl-1′-oxycarbonyl)anilino]-1,3,5-triazine (INCI name: Diethylhexyl Butamido Triazone), 2,4,6-tris[4-(2-ethylhexyl-oxycarbonyl)-anilino]-1,3,5-triazine (INCI name: Ethylhexyl Triazone), (1,3,5)-triazine-2,4-bis((4-2-ethyl-hexyloxy)-2-hydroxy)-phenyl)-6-(4-methoxy-phenyl) (bemotrizinol), octyl triazone (CAS-no.: 88122-99-0), 2,4-bis(2,4-dihydroxyphenyl)-6-(1-methyl-1H-pyrazol-5-yl)-1,3,5-triazine, 2,4-bis{[4-(2-ethylhexyloxy)-2-hydroxy]phenyl}-6-(1-methyl-1H-pyrazol-5-yl)-1,3,5-triazine, 2-[2,4-bis(2-ethylhexyloxy)phenyl]-4-[4-(2-ethylhexyloxy)-2-hydroxyphenyl]-6-(1-methyl-1H-pyrazol-5-yl)-1,3,5-triazine, 2,4-bis(2,4-dihydroxyphenyl)-6(1-phenyl-1H-pyrazol-5-yl)-1,3,5-triazine, 2,4-bis{[4-(2-ethylhexyloxy)-2-hydroxy]phenyl}-6-(1-phenyl-1H-pyrazol-5-yl)-1,3,5-triazine, 2,4-bis[4-(butoxycarbonyl)phenylamino]-6-(1-methyl-1H-pyrazol-5-yl)-1,3,5-triazine, 2,4-bis{4-[(2-(ethylhexyloxy)carbonyl]phenylamino}-6-(1-methyl-1H-pyrazol-5-yl)-1,3,5-triazine, 2,4-bis[4-(butoxycarbonyl)phenylamino]-6-(1-phenyl-1H-pyrazol-5-yl)-1,3,5-triazine, 2,4-bis{4-[(2-ethylhexyloxy)carbonyl]phenylamino}-6-(1-phenyl-1H-pyrazol-5-yl)-1,3,5-triazine and 2-[(4-butoxycarbonyl)phenylamino]-4,6-bis(1-methyl-1H-pyrazol-5-yl)-1,3,5-triazine, 2-(1-benzyl-1H-pyrrole-2-yl)-4,6-bis(2,4-dihydroxyphenyl)-1,3,5-triazine, 2-(1-benzyl-1H-pyrrole-2-yl)-4,6-bis[4-(2-ethylhexyloxy)-2-hydroxyphenyl]-1,3,5-triazine, 2-(1-benzyl-1H-pyrrole-2-yl)-4,6-bis[4-(butoxycarbonyl)phenylamino]-1,3,5-triazine, 2-(1-benzyl-1H-pyrrole-2-yl)-4,6-bis(biphenyl-4-ylamino)-1,3,5-triazine, 2-(1-benzyl-1H-pyrrole-2-yl)-4,6-bis(4-benzoylphenylamino)-1,3,5-triazine, 2-(1-benzyl-1H-pyrrole-2-yl)-4,6-bis(9-oxo-9H-fluoren-3-ylamino)-1,3,5-triazine, 2-(1-benzyl-1H-pyrrole-2-yl)-4,6-bis[4-(imidazo[1,2-a]pyridin-2-yl)phenylamino]-1,3,5-triazine, 2-(1-benzhydryl-1H-pyrrole-2-yl)-4,6-bis[4-(butoxycarbonyl)phenylamino]-1,3,5-triazine, 2-(1-benzyl-1H-pyrrole-2-yl)-4,6-bis[4-(6,6-dimethyl-4-oxo-4,5,6,7-tetrahydroindol-1-yl)phenylamino]-1,3,5-triazine, 2-(1-benzyl-1H-pyrrole-2-yl)-4,6-bis[4-((2-ethylhexyloxy)carbonyl)phenylamino]-1,3,5-triazine, 2-(1-benzyl-1H-pyrrole-2-yl)-4,6-bis[4-1H-imidazo[1,2-a]pyridin-2-yl)phenylamino]-1,3,5-triazine, 2-(1-benzyl-1H-pyrrole-2-yl)-4,6-bis[4-(1H-benzo[d]imidazol-2-yl)phenylamino]-1,3,5-triazine, 2-(1-benzyl-1H-pyrrole-2-yl)-4,6-bis[4-(1H-pyrazol-1-yl)phenylamino]-1,3,5-triazine, 2-(1-benzyl-1H-pyrrole-2-yl)-4,6-bis[naphthalen-2-ylamino]-1,3,5-triazine, 2-(1-benzyl-1H-pyrrole-2-yl)-4,6-bis[4-((E)-3-ethoxy-3-oxoprop-1-enyl)phenylamino]-1,3,5-triazine, 2-(1-benzyl-1H-pyrrole-2-yl)-4,6-bis[4-(E)-styrylphenylamino]-1,3,5-triazine, urocanic acid, ethyl-urocanic acid, talc, kaolin, calcium carbonate, bisoctrizole (CAS-no.: 103597-45-I), bornelone (CAS-no.: 2226-11-1), 2-Hydroxy-1,4-naphthochinone (Lawsone, CAS-no.: 83-72-7), petrolatum red, phenol sulphonate, sodium 3,4-dimethylphenyl-glyoxylate, 3,3′-(1,4-phenylenedimethylene-bis-(7,7-dimethyl-2-oxobicyclo-[2,2,1]hept-1-yl-methanesulfonic acid, α-(2-oxoborn-3-ylidene)-toluene-4-sulphonic acid, 2-(2H-benzotriazole-2-yl)-4-methyl-6-[2-methyl-3-[1,3,3,3-tetramethyl-1-[(trimethylsilyl)oxy]disiloxanyl]propyl]-phenol (INCI name: drometrizole trisiloxane, DTS), digalloyl trioleate, dimethicone/PEG-15 crosspolymer, ethylhexyl-bis-isopentyl-benzoxazolyl-phenyl melamine and 2-ethylhexyl-dimethoxy-benzylidene-dioxo-imidazolidine propionate;
   and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof;
   wherein the aforementioned UV-absorbing active substances (B) have their respective absorption maximum λ_{max(B)} in the range of 290 to 400 nm, preferably with the proviso that λ_{max(B)} of the respective active substance (B) does not lie in the same UV radiation range wherein the λ_{max(A)} of active substance (A) lies.

According to a further preferred embodiment of the present invention the UV-absorbing active substance (A) is at least one heptaazaphenalene compound selected from the group comprising
2,5,8-tris-(4-(butoxycarbonyl)phenylamino)-1,3,4,6,7,9,9b-heptaazaphenalene,
2,5,8-tris-(4-(2-ethylhexyloxycarbonyl)phenylamino)-1,3,4,6,7,9,9b-heptaazaphenalene,
2,5,8-tris-(4-(imidazo[1,2-a]pyridin-2-yl)-phenylamino)-1,3,4,6,7,9,9b-heptaazaphenalene,
2,5,8-tris-(4-(4,5,6,7-tetrahydro-2,6,6-trimethyl-4-oxo indol-1-yl)phenylamine)-1,3,4,6,7,9,9b-heptaazaphenalene,
2,5,8-tris-(biphenyl-4-ylamino)-1,3,4,6,7,9,9b-heptaazaphenalene,
2,5,8-tris-(4-(1H-benzo[d]imidazol-2-yl)phenylamino)-1,3,4,6,7,9,9b-heptaazaphenalene,
2,5-bis-(biphenyl-4-ylamino)-8-(4-(butoxycarbonyl)phenylamino)-1,3,4,6,7,9,9b-heptaazaphenalene,
2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene,
2,5,8-tris-(2,4-dihydroxyphenyl)-1,3,4,6,7,9,9b-heptaazaphenalene,
2,5-dichloro-8-(1-methyl-1H-pyrrole-2-yl)-1,3,4,6,7,9,9b-heptaazaphenalene,
2,5-bis-(4-(butoxycarbonyl)phenylamino)-8-(2-(1-methyl-1H-pyrrole-2-yl)-1,3,4,6,7,9,9b-heptaazaphenalene,
2-(4-(carboxy)phenylamino)-5,8-bis-(4-methylphenyl)-1,3,4,6,7,9,9b-heptaazaphenalene,
2,5,8-tris-(4-(4,5,6,7-tetrahydro-2,6,6-trimethyl-4-oxo indol-1-yl)phenylamine)-1,3,4,6,7,9,9b-heptaazaphenalene,
2,5-bis-(4-(butoxycarbonyl)phenylamino)-8-(1-methyl-1H-pyrrole-2-yl)-1,3,4,6,7,9,9b-heptaazaphenalene,
2,5-bis(2,4-dihydroxyphenyl)-8-(1-methyl-1H-pyrazol-5-yl)-1,3,4,6,7,9,9b-heptaazaphenalene,
2,5-bis{[4-(2-ethylhexyloxy)-2-hydroxy]phenyl}-6-(1-methyl-1H-pyrazol-5-yl)-1,3,4,6,7,9,9b-heptaazaphenalene,
2-[2,4-bis(2-ethylhexyloxy)phenyl]-5-[4-(2-ethylhexyl oxy)-2-hydroxyphenyl]-8-(1-methyl-1H-pyrazol-5-yl)-1,3,4,6,7,9,9b-heptaazaphenalene,
2,5-bis(2,4-dihydroxyphenyl)-8-(1-phenyl-1H-pyrazol-5-yl)-1,3,4,6,7,9,9b-heptaazaphenalene,
2,5-bis{[4-(2-ethylhexyloxy)-2-hydroxy]phenyl}-8-(1-phenyl-1H-pyrazol-5-yl)-1,3,4,6,7,9,9b-heptaazaphenalene,
2,5-bis[4-(butoxycarbonyl)phenylamino]-8-(1-methyl-1H-pyrazol-5-yl)-1,3,4,6,7,9,9b-heptaazaphenalene,
2,5-bis{4-[(2-(ethylhexyloxy)carbonyl]phenylamino}-8-(1-methyl-1H-pyrazol-5-yl)-1,3,4,6,7,9,9b-heptaazaphenalene,
2,5-bis[4-(butoxycarbonyl)phenylamino]-8-(1-phenyl-1H-pyrazol-5-yl)-1,3,4,6,7,9,9b-heptaazaphenalene,
2,5-bis{4-[(2-ethylhexyloxy)carbonyl]phenylamino}-8-(1-phenyl-1H-pyrazol-5-yl)-1,3,4,6,7,9,9b-heptaazaphenalene,
2-(1-benzyl-1H-pyrrole-2-yl)-5,8-bis(2,4-dihydroxyphenyl)-1,3,4,6,7,9,9b-heptaazaphenalene,
2-(1-benzyl-1H-pyrrole-2-yl)-5,8-bis[4-(2-ethylhexyloxy)-2-hydroxyphenyl]-1,3,4,6,7,9,9b-heptaazaphenalene,
2-(1-benzyl-1H-pyrrole-2-yl)-5,8-bis[4-(butoxycarbonyl)phenylamino]-1,3,4,6,7,9,9b-heptaazaphenalene,
2-(1-benzyl-1H-pyrrole-2-yl)-5,8-bis(biphenyl-4-ylamino)-1,3,4,6,7,9,9b-heptaazaphenalene,
2-(1-benzyl-1H-pyrrole-2-yl)-5,8-bis(4-benzoylphenylamino)-1,3,4,6,7,9,9b-heptaazaphenalene,
2-(1-benzyl-1H-pyrrole-2-yl)-5,8-bis(9-oxo-9H-fluoren-3-ylamino)-1,3,4,6,7,9,9b-heptaazaphenalene,
2-(4-(tert-butylcarbamoyl)phenylamino)-5,8-bis-(4-(2-ethylhexyloxycarbonyl)phenylamino)-1,3,4,6,7,9,9b-heptaazaphenalene,
2,5-bis-[(4-(2-ethylhexyloxy)-2-hydroxy)-phenyl]-8-(4-methoxyphenyl)-1,3,4,6,7,9,9b-heptaazaphenalene,
2-(2-ethylhexylamino)-5,8-bis-(4-(5-(1,1-dimethylpropyl)benzo[d]oxazol-2-yl)phenylamino)-1,3,4,6,7,9,9b-heptaazaphenalene,
2,5,8-tris-(4-(1H-pyrazol-1-yl)phenylamino)-1,3,4,6,7,9,9b-heptaazaphenalene
2,5,8-tris-(4-benzoylphenylamino)-1,3,4,6,7,9,9b-heptaazaphenalene,
2,5,8-tris-(4-butoxy-2-hydroxyphenyl)-1,3,4,6,7,9,9b-heptaazaphenalene,
2,5,8-tris-(naphthalen-2-ylamino)-1,3,4,6,7,9,9b-heptaazaphenalene,
2,5-bis-(4-(butoxycarbonyl)phenylamino)-8-(2-(1-methyl-1H-indol-3-yl)-1,3,4,6,7,9,9b-heptaazaphenalene,
2,5,8-tris-(biphenyl-4-yloxy)-1,3,4,6,7,9,9b-heptaazaphenalene,
2,5,8-tris-(3-methoxyphenylamino)-1,3,4,6,7,9,9b-heptaazaphenalene,
2,5,8-tris-(4-methoxyphenylamino)-1,3,4,6,7,9,9b-heptaazaphenalene,
2,5,8-tris-(4-((E)-3-ethoxy-3-oxoprop-1-enyl)phenylamino)-1,3,4,6,7,9,9b-heptaazaphenalene,
2,5,8-tris-(methoxycarbonyl-4′-biphenyl-4-ylamino)-1,3,4,6,7,9,9b-heptaazaphenalene,
2,5,8-tris-(4-(methoxycarbonyl)phenylamino)-1,3,4,6,7,9,9b-heptaazaphenalene,
2,5,8-tris-(4-(1H-benzo[d]imidazol-2-yl)-3-hydroxyphenylamino)-1,3,4,6,7,9,9b-heptaazaphenalene,
2,5,8-tris-(4-(phenylamino)phenylamino)-1,3,4,6,7,9,9b-heptaazaphenalene,
2,5,8-tris-((4-(E)-styrylphenyl)amino)-1,3,4,6,7,9,9b-heptaazaphenalene,
2,5,8-tris-(2-ethylhexylamino)-1,3,4,6,7,9,9b-heptaazaphenalene,
2,5,8-tris-(4-(L-menthylcarbonyl)phenylamino)-1,3,4,6,7,9,9b-heptaazaphenalene,
2,5,8-tris-(4-((3,3,5-trimethylcyclohexyloxy)carbonyl)phenylamino)-1,3,4,6,7,9,9b-heptaazaphenalene,
2,5,8-tris-(4-((E)-3-(2-ethylhexyloxy)-3-oxoprop-1-enyl)phenylamino)-1,3,4,6,7,9,9b-heptaazaphenalene,
2-(3-((E)-3-(2-ethylhexyloxy)-3-oxoprop-1-enyl)phenylamino)-5,8-bis-(4-((E)-3-(2-ethylhexyloxy)-3-oxoprop-1-enyl)phenylamino)-1,3,4,6,7,9,9b-heptaazaphenalene,
2,5,8-tris-(3-nitrophenylamino)-1,3,4,6,7,9,9b-heptaazaphenalene,
2,5,8-tris-(4-(2-butyloctyloxycarbonyl)phenylamino)-1,3,4,6,7,9,9b-heptaazaphenalene,
2,5,8-tris-(4-(2-hexyldecyloxycarbonyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(hexyldecyloxycarbonyl)phenylamino)-1,3,4,6,7,9,9b-heptaazaphenalene,
2,5,8-tris-(4-(octyldecyloxycarbonyl)phenylamino)-1,3,4,6,7,9,9b-heptaazaphenalene,
2,5,8-tris-((3-(E)-styrylphenyl)amino)-1,3,4,6,7,9,9b-heptaazaphenalene,
2,5,8-tris-(4-((3,5,5-trimethylhexyloxy)carbonylphenylamino)-1,3,4,6,7,9,9b-heptaazaphenalene,
2,5-bis-(3-(methoxy)phenylamino)-8-(2-(1-methyl-1H-pyrrole-2-yl)-1,3,4,6,7,9,9b-heptaazaphenalene,
2,5,8-tris-(4-((2-ethylhexyl)carbamoyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(1,3,3-trimethylbicyclo[2.2.1]heptan-2-yloxy)phenylamino)-1,3,4,6,7,9,9b-heptaazaphenalene,
2,5,8-tris-(1H-indol-5-ylamino)-1,3,4,6,7,9,9b-heptaazaphenalene,
2,5,8-tris-(4-((3,7-dimethyloctyloxy)carbonyl)phenylamino)-1,3,4,6,7,9,9b-heptaazaphenalene and
2,5,8-tris-(2-amino-4,5-dimethylphenylamino)-1,3,4,6,7,9,9b-heptaazaphenalene;
   and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof;
   which active substance (A) has its absorption maximum λ_{max(A)} in the range of either UV-A or UV-B radiation;
   and the UV-absorbing active substance (B)
   is at least one inorganic compound selected from the group comprising cerium oxide, iron oxide, manganese oxide, titanium dioxide, zinc oxide, zinc cerium oxide and zirconium oxide;
   and/or
   is at least one UV-absorbing organic compound selected from the group comprising 2,4-bis(2,4-dihydroxyphenyl)-6-(1-methyl-1H-pyrazol-5-yl)-1,3,5-triazine, 2,4-bis{[4-(2-ethylhexyloxy)-2-hydroxy]phenyl}-6-(1-methyl-1H-pyrazol-5-yl)-1,3,5-triazine, 2-[2,4-bis(2-ethylhexyloxy)phenyl]-4-[4-(2-ethylhexyloxy)-2-hydroxyphenyl]-6-(1-methyl-1H-pyrazol-5-yl)-1,3,5-triazine, 2,4-bis(2,4-dihydroxyphenyl)-6(1-phenyl-1H-pyrazol-5-yl)-1,3,5-triazine, 2,4-bis{[4-(2-ethylhexyloxy)-2-hydroxy]phenyl}-6-(1-phenyl-1H-pyrazol-5-yl)-1,3,5-triazine, 2,4-bis[4-(butoxycarbonyl)phenylamino]-6-(1-methyl-1H-pyrazol-5-yl)-1,3,5-triazine, 2,4-bis{4-[(2-(ethylhexyloxy)carbonyl]phenylamino}-6-(1-methyl-1H-pyrazol-5-yl)-1,3,5-triazine, 2,4-bis[4-(butoxycarbonyl)phenylamino]-6-(1-phenyl-1H-pyrazol-5-yl)-1,3,5-triazine, 2,4-bis{4-[(2-ethylhexyloxy)carbonyl]phenylamino}-6-(1-phenyl-1H-pyrazol-5-yl)-1,3,5-triazine and 2-[(4-butoxycarbonyl)phenylamino]-4,6-bis(1-methyl-1H-pyrazol-5-yl)-1,3,5-triazine, 2-(1-benzyl-1H-pyrrole-2-yl)-4,6-bis(2,4-dihydroxyphenyl)-1,3,5-triazine, 2-(1-benzyl-1H-pyrrole-2-yl)-4,6-bis[4-(2-ethylhexyloxy)-2-hydroxyphenyl]-1,3,5-triazine, 2-(1-benzyl-1H-pyrrole-2-yl)-4,6-bis[4-(butoxycarbonyl)phenylamino]-1,3,5-triazine, 2-(1-benzyl-1H-pyrrole-2-yl)-4,6-bis(biphenyl-4-ylamino)-1,3,5-triazine, 2-(1-benzyl-1H-pyrrole-2-yl)-4,6-bis(4-benzoylphenylamino)-1,3,5-triazine, 2-(1-benzyl-1H-pyrrole-2-yl)-4,6-bis(9-oxo-9H-fluoren-3-ylamino)-1,3,5-triazine, 2-(1-benzyl-1H-pyrrole-2-yl)-4,6-bis[4-(imidazo[1,2-a]pyridin-2-yl)phenylamino]-1,3,5-triazine, 2-(1-benzhydryl-1H-pyrrole-2-yl)-4,6-bis[4-(butoxycarbonyl)phenylamino]-1,3,5-triazine, 2-(1-benzyl-1H-pyrrole-2-yl)-4,6-bis[4-(6,6-dimethyl-4-oxo-4,5,6,7-tetrahydroindol-1-yl)phenylamino]-1,3,5-triazine, 2-(1-benzyl-1H-pyrrole-2-yl)-4,6-bis[4-((2-ethylhexyloxy)carbonyl)phenylamino]-1,3,5-triazine, 2-(1-benzyl-1H-pyrrole-2-yl)-4,6-bis[4-1H-imidazo[1,2-a]pyridin-2-yl)phenylamino]-1,3,5-triazine, 2-(1-benzyl-1H-pyrrole-2-yl)-4,6-bis[4-(1H-benzo[d]imidazol-2-yl)phenylamino]-1,3,5-triazine, 2-(1-benzyl-1H-pyrrole-2-yl)-4,6-bis[4-(1H-pyrazol-1-yl)phenylamino]-1,3,5-triazine, 2-(1-benzyl-1H-pyrrole-2-yl)-4,6-bis[naphthalen-2-ylamino]-1,3,5-triazine, 2-(1-benzyl-1H-pyrrole-2-yl)-4,6-bis[4-((E)-3-ethoxy-3-oxoprop-1-enyl)phenylamino]-1,3,5-triazine, 2-(1-benzyl-1H-pyrrole-2-yl)-4,6-bis[4-(E)-styrylphenylamino]-1,3,5-triazine,

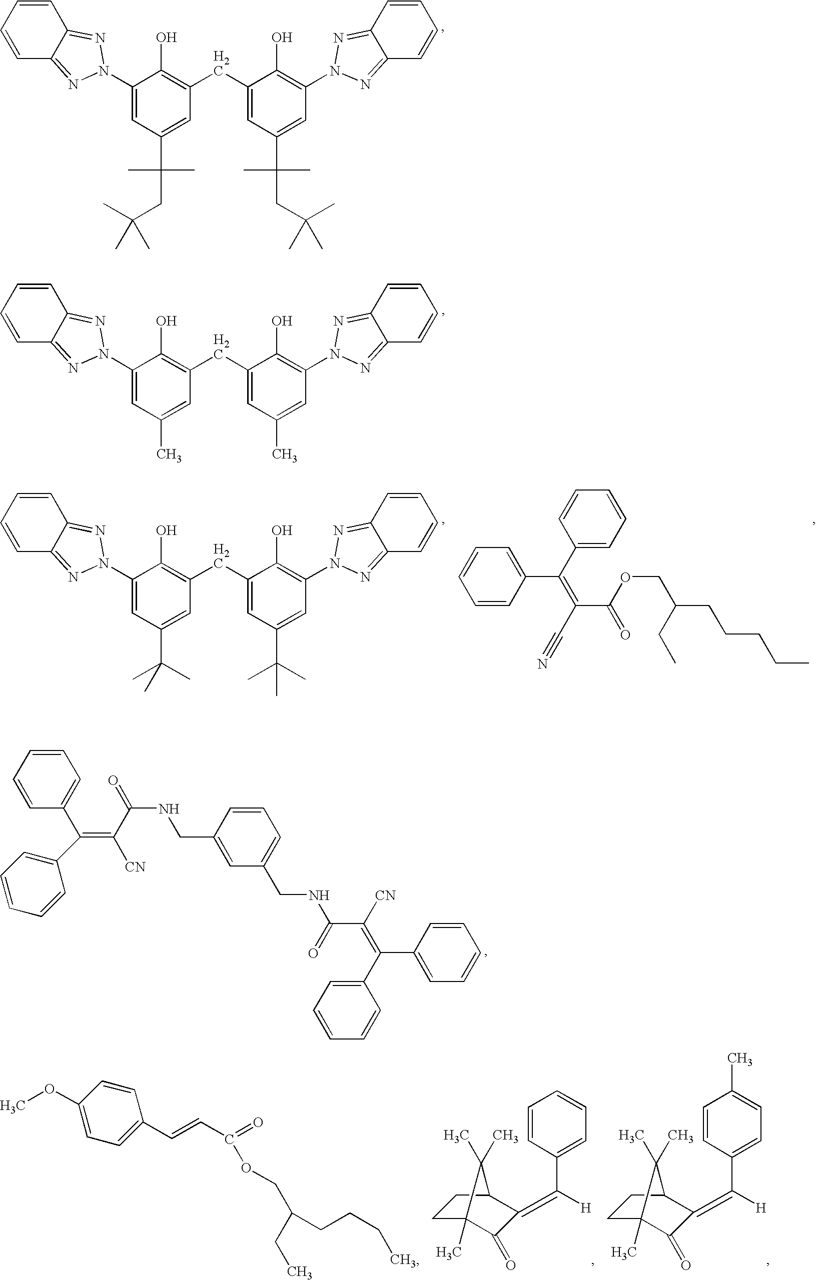

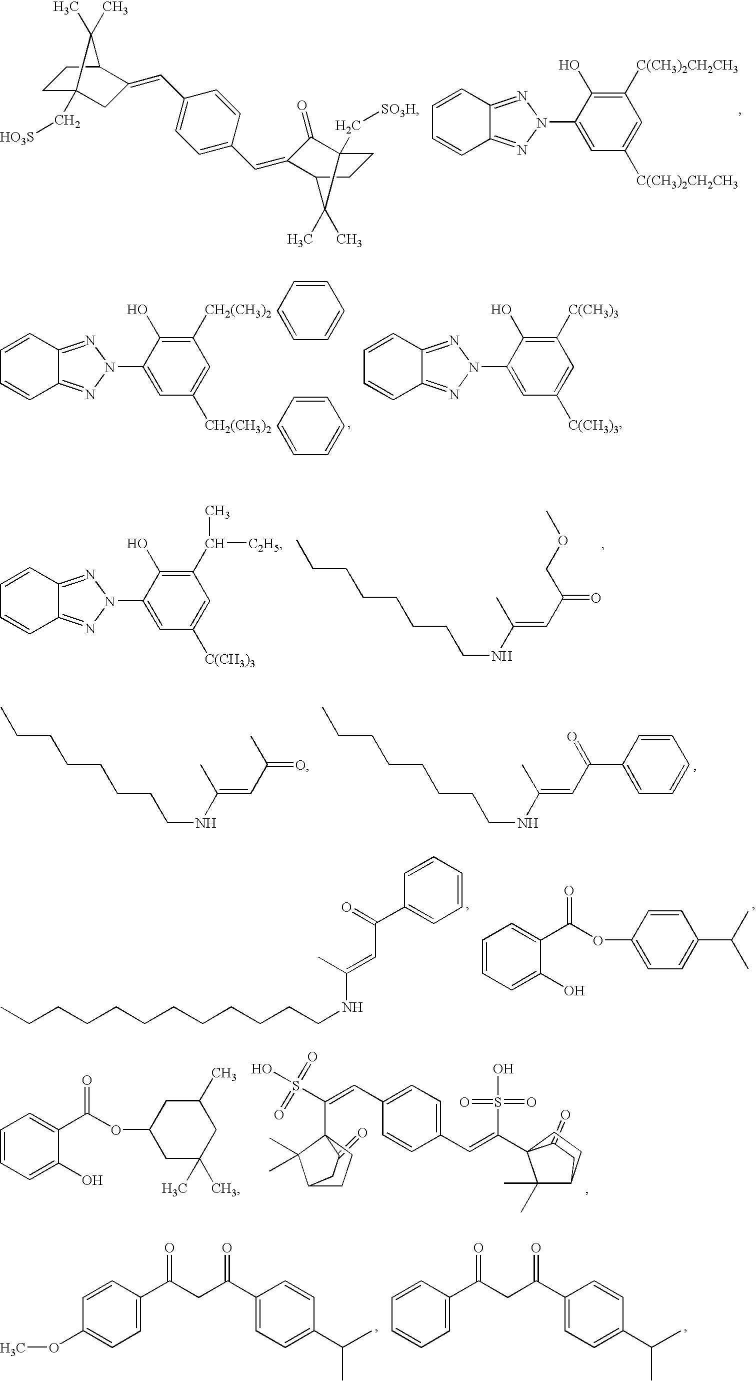

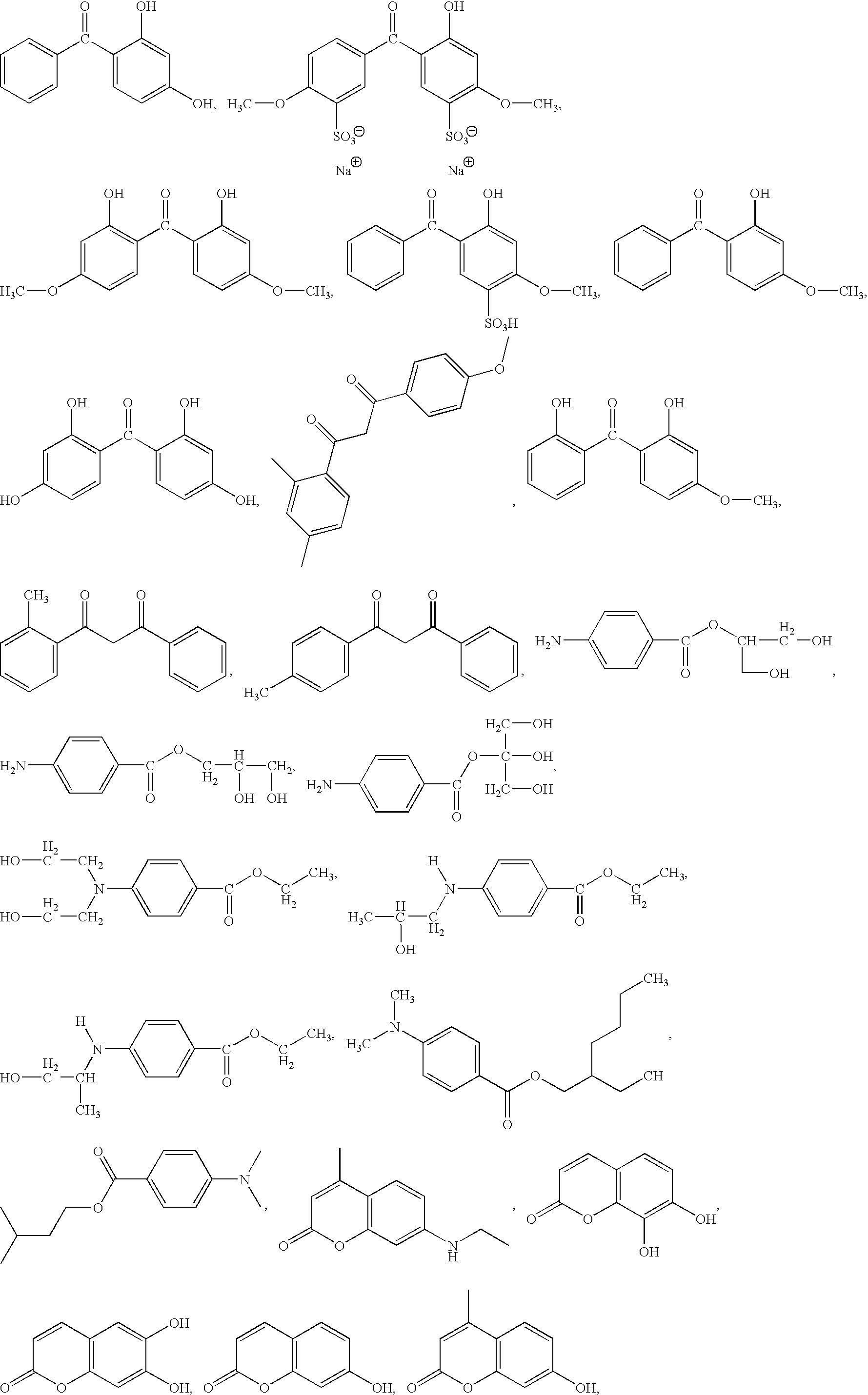

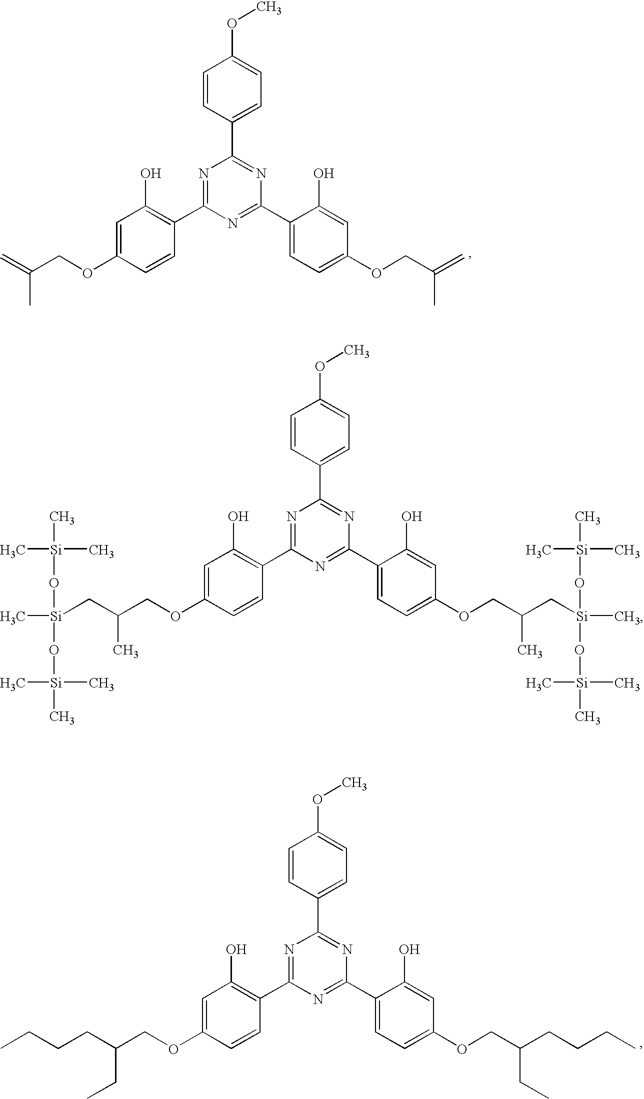

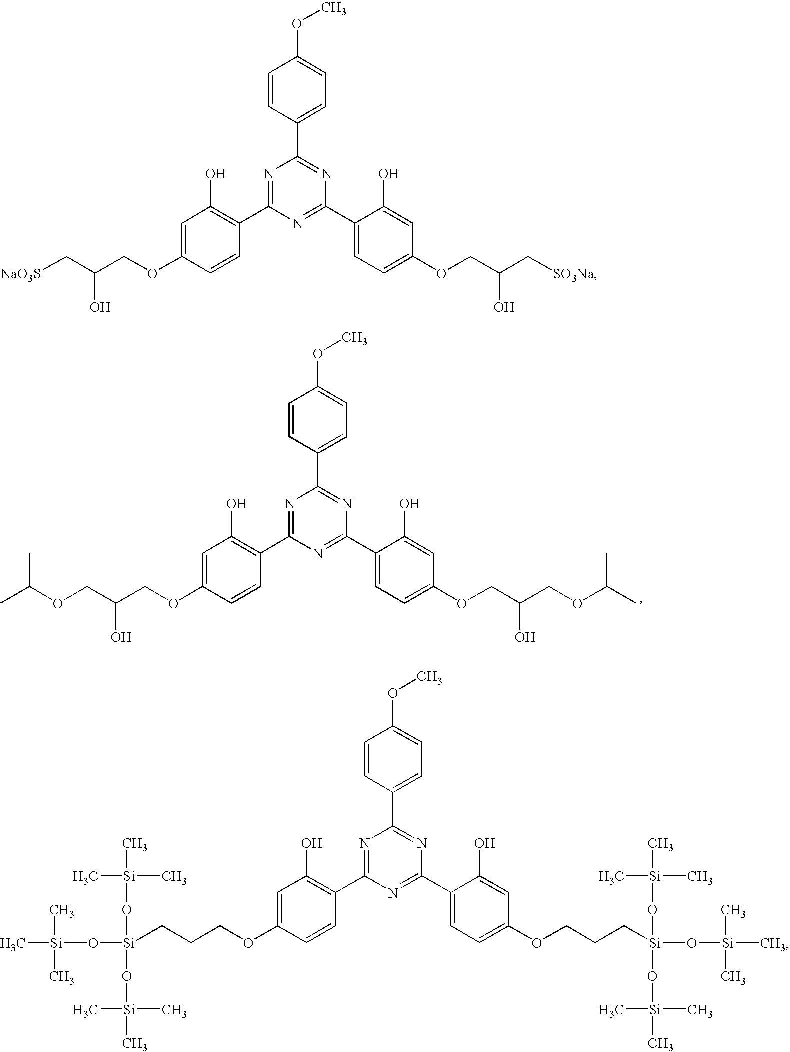

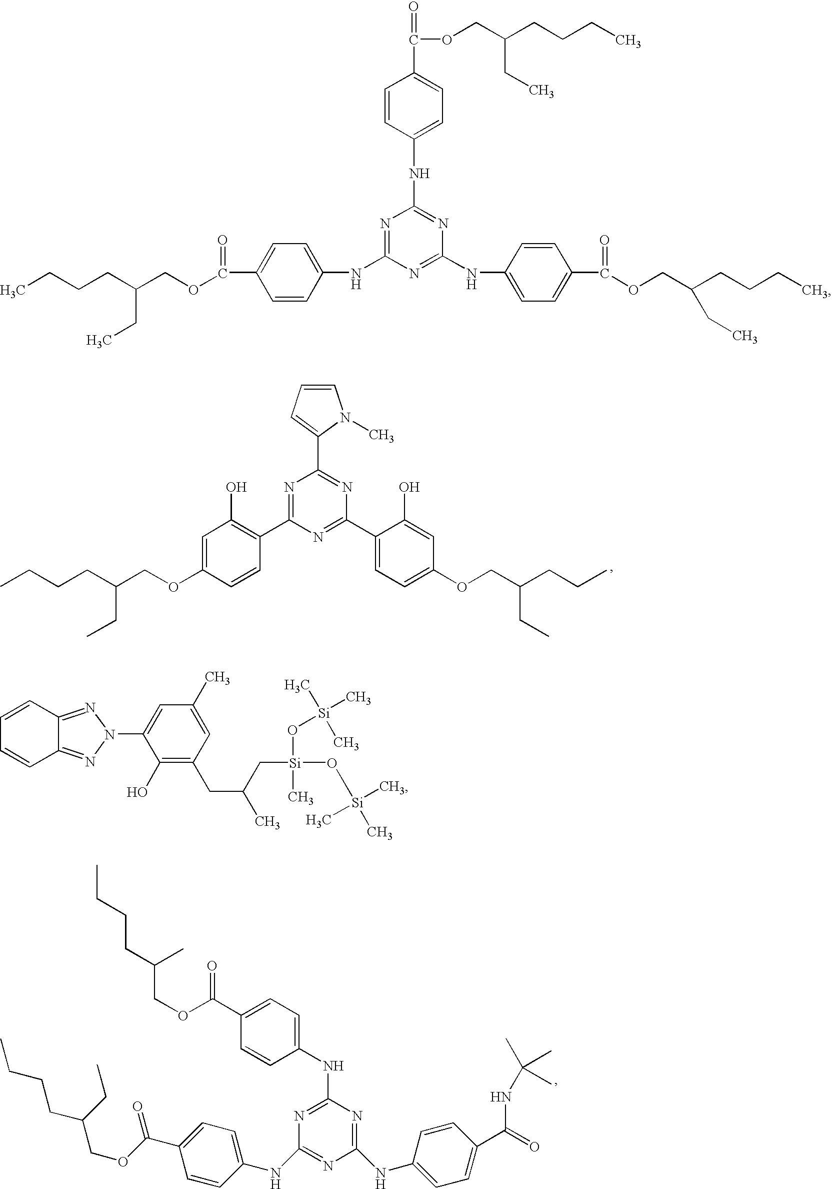

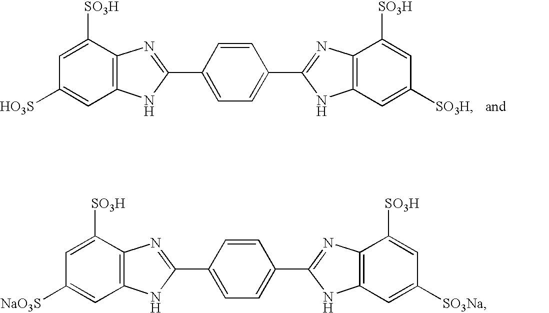

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof;
wherein the aforementioned UV-absorbing active substances (B) have their respective absorption maximum λ_{max(B)} in the range of 290 to 400 nm, preferably with the proviso that λ_{max(B)} of the respective active substance (B) does not lie in the same UV radiation range wherein the λ_{max(A)} of active substance (A) lies.

A particularly preferred embodiment of the present invention is a combination of the UV-absorbing active substance (A)
(1) 2,5,8-tris-(4-(butoxycarbonyl)phenylamino)-1,3,4,6,7,9,9b-heptaazaphenalene

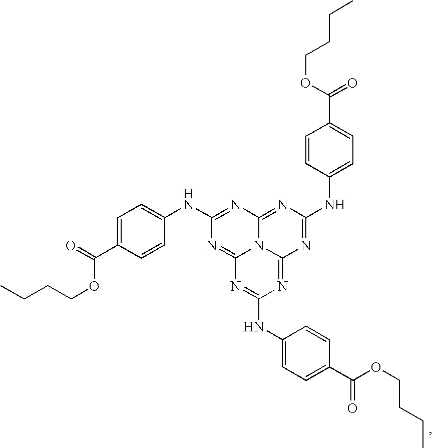

and the UV-absorbing active substance (B)
is at least one compound selected from the group consisting of 2-ethylhexyl 2-cyano-3,3′-diphenylacrylate, ethyl 2-cyano-3,3′-diphenylacrylate, polymer of N-{(2 and 4)[(2-oxoborn-3-ylidene)methyl]benzyl}acrylamide, methyl 4-aminobenzoate, butyl 4-aminobenzoate, 5-methyl-2-(1-methylethyl)-cyclohexyl 2-aminobenzoate, glyceryl aminobenzoate, 2-ethylhexyl-p-dimethylethyl-aminobenzoate, 4-aminobenzamide, homomethyl-N-acetyl-anthranilic acid, menthyl antrhanilate, 2-(1H-benzimidazol-2-yl)-4-methoxyphenol, 2,2′-bis(benzimidazole), monosodium salt of 2-2′-bis-(1,4-phenylene)1H-benzimidazole-4,6-disulphonic acid, 2-phenylbenzimidazole-5-sulphonic acid, 5,5′,6,6′-tetramethyl-2,2′-bis(benzimidazole), 5,5′-dimethyl-2,2′-bis(benzimidazole), 6-methoxy-2,2′-bis(benzimidazole), 1,4-phenylenebis(2-benzimidazolyl), 1,2-phenylenebis)benzimidazolyl), 1,4-phenylenebis(N-2-ethylhexyl-2-benzimidazolyl), 1,4-phenylenebis(N-tri-methylsilylmethyl-2-benzimidazolyl), 2-(1H-benzimidazol-2-yl)benzothiazole, 2-(1H-benzimidazol-2-yl)benzoxazole, N,N′-dimethyl-2,2′-bis(benzimidazole), phenylbenzimidazole-5-sulfonic acid (Ensulizole) and its salts, 2-2′-bis-(1,4-phenylene)1H-benzimidazole-4,6-disulphonic acid, 3-methylbutyl 4-(dimethylamino)benzoate, 3,3,5-trimethylcyclohexyl 2-hydroxybenzoate (Homosalate), ethyl 4-aminobenzoate, 4,4-((6-(((1,1-dimethylethyl)amino)carbonyl)phenyl)amino) 1,3,5-triazine-2,4-diyl)diimino)bis-,bis-(2-ethyl-hexyl)ester) benzoic acid, 2,4-dihydroxy-benzophenone (Benzophenone-1), 2,2′-dihydroxy-4-methoxy-benzophenone (dioxybenzone, Benzophenone-8), 2,2′-dihydroxy-4,4′-dimethoxy-benzophenone, 2,2′,4,4′-tetrahydroxy-benzophenone, 2-hydroxy-4-methoxy-4′-methyl-benzophenone, hydroxymethoxy-benzophenone sulfonic acid, (2-Hydroxy-4-methoxyphenyl)-phenylmethanone (Benzophenone-3), 2-hydroxy-4-methoxy-benzophenone-5-sulfonic acid (Benzophenone-4) and its salts, 4-phenyl-benzophenone, 2-ethylhexyl-4′-phenyl-benzophenone-2-carboxylate, 2-hydroxy-4-n-octoxy-benzophenone, 4-hydroxy-3-carboxy-benzophenone, 2-hydroxy-4-methoxybenzophenone, 4-(2-β-glucopyranosiloxy)-propoxy-2-hydroxybenzophenone, dihydroxy-dimethoxy-disulfobenzophenone, 2-benzothiazol-2-ylphenol, [2,4′-dihydroxy-3-(2H-benzotriazol-2-yl)-5-(1,1,3,3-tetramethylbutyl)-2′-n-octoxy-5′-benzoyl]-diphenylmethane, 2,2′-hydroxy-5-methylphenyl-benzotriazole, 2-(2′-hydroxy-5′-t-octylphenyl)benzotriazole, 2-(2′-hydroxy-5′-methylphenyl)benzotriazole, 2,2′-methylene-bis-6-(2H-benzotriazol-2-yl)-4-(tetramethyl-butyl)-1,1,3,3-phenol, 2-benzoxazole-2-yl-4-methylphenol, 2-phenyl-5-methylbenzoxazole, 1,4-phenylenebis(2-benzoxazolyl), 1,3-phenylenebis(2-benzoxazolyl), 1,2-phenylenebis(2-benzoxazolyl), 2-(2-benzofuryl)-benzoxazole, 2-(benzofuryl)-5-methylbenzoxazole, 2-(3-methyl-2-benzofuryl)benzoxazole, 3-(4′-methylbenzylidene)-D,L-camphor, terephthalylidene dicamphor sulphonic acid, 3-benzylidene-D,L-camphor, 4-benzylidene-D,L-camphor, di-tert-butyl hydroxybenzylidene camphor, benzylidene camphor sulphonic acid, camphor benzalkonium methosulfate, polyacrylamido-methyl benzylidene camphor, ethylhexyl methoxycinnamate, diethanolamine p-methoxy cinnamate, isopropyl-p-acetamido cinnamate, octyl cinnamate, ethyl-4-isopropylcinnamate, methyl-2,5-diisopropyl cinnamate, ethyl-2,4-diisopropylcinnamate, methyl-2,4-diisopropylcinnamate, propyl-p-methoxycinnamate, isopropyl-p-methoxycinnamate, diisopropylcinnamate esters, isoamyl-p-methoxycinnamate, cyclohexyl-p-methoxycinnamate, ethyl-α-cyano-β-phenylcinnamate, 2-ethylhexyl-α-cyano-β-phenylcinnamate, glycerin-mono-2-ethyl-hexanoyl-diparamethoxycinnamate, glyceryl octanoate-di-p-methoxycinnamate, DEA-methoxycinnamate, methyl-bis(trimethyl-siloxy)silyl-isopentyl-trimethoxycinnamate, ethyl-α-cyano-3,5-dimethoxy-4-hydroxy cinnamate, ethyl-α-acetyl-3,5-dimethoxy-4-hydroxy cinnamate, iso-propyl-α-acetyl-3,5-dimethoxy-4-hydroxy cinnamate, iso-amyl-α-acetyl-3,5-dimethoxy-4-hydroxy cinnamate, 2-ethylhexyl-α-acetyl-3,5-dimethoxy-4-hydroxy cinnamate, ethyl-α-cyano-3-methoxy-4-hydroxy cinnamate, ethyl-α-acetyl-3-methoxy-4-hydroxy cinnamate, iso-propyl-α-acetyl-3-methoxy-4-hydroxy cinnamate, iso-amyl-α-acetyl-3-methoxy-4-hydroxy cinnamate, 2-ethylhexyl-α-acetyl-3-methoxy-4-hydroxy cinnamate, 7-ethylamino-4-methylcoumarin, 7,8-dihydroxycoumarin, 6,7-dihydroxycoumarin, 7-hydroxycoumarin, 4-methyl-7-hydroxycoumarin, diacetycurcumin, tetrahydrocurcumin diacetate, dianisoylmethane, dibenzalazine, butyl methoxydibenzoylmethane (Avobenzone), 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, ferulic acid, dimethicodiethylbenzalmalonate, diethyl-3,5-dimethoxy-4-hydroxy benzylidene malonate, di-(2-ethylhexyl)-3,5-dimethoxy-4-hydroxy benzylidene malonate, diisoamyl-3,5-dimethoxy-4-hydroxy benzylidene malonate, didodecyl-3,5-dimethoxy-4 hydroxy benzylidene malonate, dipalmitoyl-3,5-dimethoxy-4-hydroxy benzylidene malonate, di-isopropyl-3,5-dimethoxy-4-hydroxy benzylidene malonate, diethyl-3-methoxy-4-hydroxy benzylidene malonate, di-(2-ethylhexyl)-3-methoxy-4-hydroxy benzylidene malonate, diisoamyl-3-methoxy-4-hydroxy benzylidene malonate, didodecyl-3-methoxy-4-hydroxy benzylidene malonate, dipalmitoyl-3-methoxy-4-hydroxy benzylidene malonate, di-isopropyl-3-methoxy-4-hydroxy benzylidene malonate, hydrolyzed aspergillus sp. extract ferment, calcium cerium oxide, cerium oxide, iron oxide, manganese oxide, titanium dioxide, zinc oxide, zinc cerium oxide, zirconium oxide, 4-nitrobenzamide, 4-nitrobenzoic acid, p-aminobenzoic acid (PABA), PABA-monoglycerinester, N,N-dipropoxy-PABA-ethylester, N,N-diethoxy PABA-ethylester, N,N-dimethyl PABA-ethylester, N,N-dimethyl PABA-butylester, PEG-25 PABA, N,N-dimethyl PABA-amylester, N,N-dimethyl PABA-octlylester (2-ethylhexyl 4-(dimethylamino)benzoate, Padimate O), octyl dimethyl PABA, ethyl-dihydroxypropyl-PABA, isoamyl dimethyl PABA (3-methylbutyl 4-(dimethylamino)benzoate, Padimate A), 1-(3,4-dimethoxyphenyl)-4,4-dimethyl-1,3-pentanedione, 5-(3,3-Dimethyl-2-norbornyliden)-3-pentane-2-one, 1-(4-methoxyphenyl)-3-(4-tert-butylphenyl)propane-1,3-dione, dipropylene glycol salicylate, 2-ethylhexyl 2-cyano-3,3-diphenyl-2-propenoate (Octocrylene), hydrolyzed soy extract, hydrolyzed linseed extract, sunflower seed extract, grape seed extract, pinus pinaster bark extract, pinus pinaster bud extract, ginseng extract ferment, spirulina platensis powder, phenyl salicylate, p-tert-butylphenyl salicylate, p-octylphenyl salicylate, 2-ethylhexyl salicylate (octyl salicylate), amyl salicylate, menthyl salicylate, homomethyl salicylate, benzyl salicylate, p-isopropanol-phenyl salicylate, TEA-salicylate, 2-(2H-benzotriazol-2-yl)-4-methyl-6-[2-methyl-3-[1,3,3,3-tetramethyl-1-[(trimethylsilyl)oxy]disiloxanyl]propyl]-phenol (Mexoryl XL), 2-[p-(tert-butylamido)anilino]-4,6-bis[p-(2′-ethylhexyl-1′-oxycarbonyl)anilino]-1,3,5-triazine (INCI name: Diethylhexyl Butamido Triazone), 2,4,6-tris[4-(2-ethylhexyl-oxycarbonyl)-anilino]-1,3,5-triazine (INCI name: Ethylhexyl Triazone), (1,3,5)-triazine-2,4-bis((4-2-ethyl-hexyloxy)-2-hydroxy)-phenyl)-6-(4-methoxy-phenyl) (bemotrizinol), octyl triazone (CAS-no.: 88122-99-0), 2,4-bis(2,4-dihydroxyphenyl)-6-(1-methyl-1H-pyrazol-5-yl)-1,3,5-triazine, 2,4-bis{[4-(2-ethylhexyloxy)-2-hydroxy]phenyl}-6-(1-methyl-1H-pyrazol-5-yl)-1,3,5-triazine, 2-[2,4-bis(2-ethylhexyloxy)phenyl]-4-[4-(2-ethylhexyloxy)-2-hydroxyphenyl]-6-(1-methyl-1H-pyrazol-5-yl)-1,3,5-triazine, 2,4-bis(2,4-dihydroxyphenyl)-6(1-phenyl-1H-pyrazol-5-yl)-1,3,5-triazine, 2,4-bis{[4-(2-ethylhexyloxy)-2-hydroxy]phenyl}-6-(1-phenyl-1H-pyrazol-5-yl)-1,3,5-triazine, 2,4-bis [4-(butoxycarbonyl)phenylamino]-6-(1-methyl-1H-pyrazol-5-yl)-1,3,5-triazine, 2,4-bis{4-[(2-(ethylhexyloxy)carbonyl]phenylamino}-6-(1-methyl-1H-pyrazol-5-yl)-1,3,5-triazine, 2,4-bis[4-(butoxycarbonyl)phenylamino]-6-(1-phenyl-1H-pyrazol-5-yl)-1,3,5-triazine, 2,4-bis{4-[(2-ethylhexyloxy)carbonyl]phenylamino}-6-(1-phenyl-1H-pyrazol-5-yl)-1,3,5-triazine and 2-[(4-butoxycarbonyl)phenylamino]-4,6-bis(1-methyl-1H-pyrazol-5-yl)-1,3,5-triazine, 2-(1-benzyl-1H-pyrrole-2-yl)-4,6-bis(2,4-dihydroxyphenyl)-1,3,5-triazine, 2-(1-benzyl-1H-pyrrole-2-yl)-4,6-bis[4-(2-ethylhexyloxy)-2-hydroxyphenyl]-1,3,5-triazine, 2-(1-benzyl-1H-pyrrole-2-yl)-4,6-bis[4-(butoxycarbonyl)phenylamino]-1,3,5-triazine, 2-(1-benzyl-1H-pyrrole-2-yl)-4,6-bis(biphenyl-4-ylamino)-1,3,5-triazine, 2-(1-benzyl-1H-pyrrole-2-yl)-4,6-bis(4-benzoylphenylamino)-1,3,5-triazine, 2-(1-benzyl-1H-pyrrole-2-yl)-4,6-bis(9-oxo-9H-fluoren-3-ylamino)-1,3,5-triazine, 2-(1-benzyl-1H-pyrrole-2-yl)-4,6-bis[4-(imidazo[1,2-a]pyridin-2-yl)phenylamino]-1,3,5-triazine, 2-(1-benzhydryl-1H-pyrrole-2-yl)-4,6-bis[4-(butoxycarbonyl)phenylamino]-1,3,5-triazine, 2-(1-benzyl-1H-pyrrole-2-yl)-4,6-bis[4-(6,6-dimethyl-4-oxo-4,5,6,7-tetrahydroindol-1-yl)phenylamino]-1,3,5-triazine, 2-(1-benzyl-1H-pyrrole-2-yl)-4,6-bis[4-((2-ethylhexyloxy)carbonyl)phenylamino]-1,3,5-triazine, 2-(1-benzyl-1H-pyrrole-2-yl)-4,6-bis[4-1H-imidazo[1,2-a]pyridin-2-yl)phenylamino]-1,3,5-triazine, 2-(1-benzyl-1H-pyrrole-2-yl)-4,6-bis[4-(1H-benzo[d]imidazol-2-yl)phenylamino]-1,3,5-triazine, 2-(1-benzyl-1H-pyrrole-2-yl)-4,6-bis[4-(1H-pyrazol-1-yl)phenylamino]-1,3,5-triazine, 2-(1-benzyl-1H-pyrrole-2-yl)-4,6-bis[naphthalen-2-ylamino]-1,3,5-triazine, 2-(1-benzyl-1H-pyrrole-2-yl)-4,6-bis[4-((E)-3-ethoxy-3-oxoprop-1-enyl)phenylamino]-1,3,5-triazine, 2-(1-benzyl-1H-pyrrole-2-yl)-4,6-bis[4-(E)-styrylphenylamino]-1,3,5-triazine, urocanic acid, ethyl-urocanic acid, talc, kaolin, calcium carbonate, bisoctrizole (CAS-no.: 103597-45-I), bornelone (CAS-no.: 2226-11-1), 2-Hydroxy-1,4-naphthochinone (Lawsone, CAS-no.: 83-72-7), petrolatum red, phenol sulphonate, sodium 3,4-dimethylphenyl-glyoxylate, 3,3′-(1,4-phenylenedimethylene-bis-(7,7-dimethyl-2-oxobicyclo-[2,2,1]hept-1-yl-methanesulfonic acid, α-(2-oxoborn-3-ylidene)-toluene-4-sulphonic acid, 2-(2H-benzotriazole-2-yl)-4-methyl-6-[2-methyl-3-[1,3,3,3-tetramethyl-1-[(trimethylsilyl)oxy]disiloxanyl]propyl]-phenol (INCI name: drometrizole trisiloxane, DTS), digalloyl trioleate, dimethicone/PEG-15 crosspolymer, ethylhexyl-bis-isopentyl-benzoxazolyl-phenyl melamine and 2-ethylhexyl-dimethoxy-benzylidene-dioxo-imidazolidine propionate;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof;
with the exception of the combination of 37.5% by weight, based on the weight of the combination, of 2,5,8-tris-(4-(butoxycarbonyl)phenylamino)-1,3,4,6,7,9,9b-heptaazaphenalene and 62.5% by weight, based on the weight of the combination, of ethylhexyl methoxycinnamate.

Another preferred embodiment of the present invention is a combination of the UV-absorbing active substance (A)
(2) 2,5,8-tris-(4-(2-ethylhexyloxycarbonyl)phenylamino)-1,3,4,6,7,9,9b-heptaazaphenalene

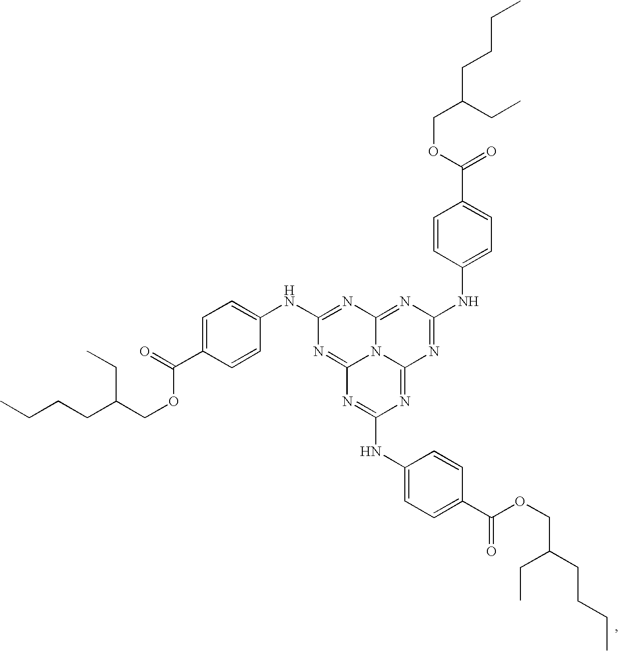

and the UV-absorbing active substance (B)
is at least one compound selected from the before mentioned groups cited in relation to the combination of 2,5,8-tris-(4-(butoxycarbonyl)phenylamino)-1,3,4,6,7,9,9b-heptaazaphenalene;
with the exception of the combination of 60.5 to 67.6% by weight, preferably 60 to 70% by weight, based on the weight of the combination, of 2,5,8-tris-(4-(2-ethylhexyloxycarbonyl)phenylamino)-1,3,4,6,7,9,9b-heptaazaphenalene and 32.4 to 39.5% by weight, preferably 30 to 40% by weight, based on the weight of the combination, of titanium dioxide.

Another preferred embodiment of the present invention is a combination of the UV-absorbing active substance (A)
(3) 2,5,8-tris-(4-(imidazo[1,2-a]pyridin-2-yl)-phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(4) 2,5,8-tris-(4-(4,5,6,7-tetrahydro-2,6,6-trimethyl-4-oxo indol-1-yl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(5) 2,5,8-tris-(biphenyl-4-ylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(6) 2,5,8-tris-(4-(1H-benzo[d]imidazol-2-yl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(7) 2,5-bis-(biphenyl-4-ylamino)-8-(4-(butoxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(8) 2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(9) 2,5,8-tris-(2,4-dihydroxyphenyl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(10) 2,5-dichloro-8-(1-methyl-1H-pyrrole-2-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(11) 2,5-bis-(4-(butoxycarbonyl)phenylamino)-8-(2-(1-methyl-1H-pyrrole-2-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(12) 2-(4-(carboxy)phenylamino)-5,8-bis-(4-methylphenyl)-1,3,4,6,7,9,9b-heptaazaphenalene,

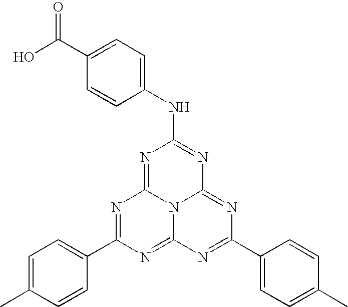

and the UV-absorbing active substance (B)
is at least one compound selected from the before mentioned groups cited in relation to the combination of 2,5,8-tris-(4-(butoxycarbonyl)phenylamino)-1,3,4,6,7,9,9b-heptaazaphenalene;
with the exception of the combination of 60.5 to 67.6% by weight, preferably 60 to 70% by weight, based on the weight of the combination, of 2-(4-(carboxy)phenylamino)-5,8-bis-(4-methylphenyl)-1,3,4,6,7,9,9b-heptaazaphenalene and 32.4 to 39.5% by weight, preferably 30 to 40% by weight, based on the weight of the combination, of titanium dioxide.

Another preferred embodiment of the present invention is a combination of the UV-absorbing active substance (A)
(13) 2,5,8-tris-(4-(4,5,6,7-tetrahydro-2,6,6-trimethyl-4-oxo indo-1-yl)phenylamine)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(14) 2,5-bis-(4-(butoxycarbonyl)phenylamino)-8-(1-methyl-1H-pyrrole-2-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(15) 2,5-bis(2,4-dihydroxyphenyl)-8-(1-methyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(16) 2,5-bis{[4-(2-ethylhexyloxy)-2-hydroxy]phenyl}-6-(1-methyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(17) 2-[2,4-bis(2-ethylhexyloxy)phenyl]-5-[4-(2-ethylhexyl oxy)-2-hydroxyphenyl]-8-(1-methyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(18) 2,5-bis(2,4-dihydroxyphenyl)-8-(1-phenyl-1H-pyrazol-5-yl)-1,3,4,6,7,9,9b-heptaazaphenalene,
   and the UV-absorbing active substance (B)
   is at least one compound at least one UV-absorbing active substance (B) selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(19) 2,5-bis{[4-(2-ethylhexyloxy)-2-hydroxy]phenyl}-8-(1-phenyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(20) 2,5-bis[4-(butoxycarbonyl)phenylamino]-8-(1-methyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(21) 2,5-bis{4-[(2-(ethylhexyloxy)carbonyl]phenylamino}-8-(1-methyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(22) 2,5-bis[4-(butoxycarbonyl)phenylamino]-8-(1-phenyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(23) 2,5-bis{4-[(2-ethylhexyloxy)carbonyl]phenylamino}-8-(1-phenyl-1H-pyrazol-5-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(24) 2-(1-benzyl-1H-pyrrole-2-yl)-5,8-bis(2,4-dihydroxyphenyl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(25) 2-(1-benzyl-1H-pyrrole-2-yl)-5,8-bis[4-(2-ethylhexyloxy)-2-hydroxyphenyl]-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(26) 2-(1-benzyl-1H-pyrrole-2-yl)-5,8-bis[4-(butoxycarbonyl)phenylamino]-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(27) 2-(1-benzyl-1H-pyrrole-2-yl)-5,8-bis(biphenyl-4-ylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(28) 2-(1-benzyl-1H-pyrrole-2-yl)-5,8-bis(4-benzoylphenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(29) 2-(1-benzyl-1H-pyrrole-2-yl)-5,8-bis(9-oxo-9H-fluoren-3-ylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(30) 2-(4-(tert-butylcarbamoyl)phenylamino)-5,8-bis-(4-(2-ethylhexyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(31) 2,5-bis-[(4-(2-ethylhexyloxy)-2-hydroxy)-phenyl]-8-(4-methoxyphenyl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(32) 2-(2-ethylhexylamino)-5,8-bis-(4-(5-(1,1-dimethylpropyl)benzo[d]oxazol-2-yl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(33) 2,5,8-tris-(4-(1H-pyrazol-1-yl)phenylamino)-1,3, 4, 6, 7, 9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(34) 2,5,8-tris-(4-benzoylphenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(35) 2,5,8-tris-(4-butoxy-2-hydroxyphenyl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(36) 2,5,8-tris-(naphthalen-2-ylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(37) 2,5-bis-(4-(butoxycarbonyl)phenylamino)-8-(2-(1-methyl-1H-indol-3-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(38) 2,5,8-tris-(biphenyl-4-yloxy)-1,3,4,6,7,9,9b-heptaazaphenalene,
   and the UV-absorbing active substance (B)
   is at least one compound) selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(39) 2,5,8-tris-(3-methoxyphenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(40) 2,5,8-tris-(4-methoxyphenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(41) 2,5,8-tris-(4-((E)-3-ethoxy-3-oxoprop-1-enyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(42) 2,5,8-tris-(methoxycarbonyl-4′-biphenyl-4-ylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(43) 2,5,8-tris-(4-(methoxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(44) 2,5,8-tris-(4-(1H-benzo[d]imidazol-2-yl)-3-hydroxyphenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(45) 2,5,8-tris-(4-(phenylamino)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(46) 2,5,8-tris-((4-(E)-styrylphenyl)amino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(47) 2,5,8-tris-(2-ethylhexylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(48) 2,5,8-tris-(4-(L-menthylcarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(49) 2,5,8-tris-(4-((3,3,5-trimethylcyclohexyloxy)carbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(50) 2,5,8-tris-(4-((E)-3-(2-ethylhexyloxy)-3-oxoprop-1-enyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(51) 2-(3-((E)-3-(2-ethylhexyloxy)-3-oxoprop-1-enyl)phenylamino)-5,8-bis-(4-((E)-3-(2-ethylhexyloxy)-3-oxoprop-1-enyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(52) 2,5,8-tris-(3-nitrophenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(53) 2,5,8-tris-(4-(2-butyloctyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(54) 2,5,8-tris-(4-(2-hexyldecyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(55) 2,5,8-tris-(4-(dodecyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(56) 2,5,8-tris-(4-(hexyldecyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(57) 2,5,8-tris-(4-(octyldecyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(58) 2,5,8-tris-((3-(E)-styrylphenyl)amino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(59) 2,5,8-tris-(4-((3,5,5-trimethylhexyloxy)carbonylphenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(60) 2,5-bis-(3-(methoxy)phenylamino)-8-(2-(1-methyl-1H-pyrrole-2-yl)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(61) 2,5,8-tris-(4-((2-ethylhexyl)carbamoyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(62) 2,5,8-tris-(4-(dodecyloxycarbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(63) 2,5,8-tris-(4-(1,3,3-trimethylbicyclo[2.2.1]heptan-2-yloxy)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(64) 2,5,8-tris-(1H-indol-5-ylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(65) 2,5,8-tris-(4-((3,7-dimethyloctyloxy)carbonyl)phenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

Another preferred embodiment of the present invention is a combination of the UV-absorbing active substance (A)
(66) 2,5,8-tris-(2-amino-4,5-dimethylphenylamino)-1,3,4,6,7,9,9b-heptaazaphenalene,
   and the UV-absorbing active substance (B)
   is at least one compound selected from the before mentioned groups cited in relation to the combination of 2,5,8-tris-(4-(butoxycarbonyl)phenylamino)-1,3,4,6,7,9,9b-heptaazaphenalene.

In all of the aforementioned 66 combinations comprising active substances (A) 1 to 66 and their respective combinations with active substances (B), the respective absorption maximum λ_{max(A)} of the respective active substances (A) is in the range of either UV-A or UV-B radiation. The respective aforementioned UV-absorbing active substances (B) in these 66 combinations have their respective absorption maximum λ_{max(B)} in the range of 290 to 400 nm, preferably with the proviso that λ_{max(B)} of the respective active substance (B) does not lie in the same UV radiation range wherein the λ_{max(A)} of active substance (A) lies.

The active substances (A) of the general formula (I), in particular, the heptaazaphenalene derivatives of general formula (I) can be obtained according to the known procedures (e.g. Shroeder, H.; Kober, E. J. Org. Chem. 1962, 27, 4262). A general scheme for obtaining heptaazaphenalene derivatives of general formula (I) is shown below (scheme 1).

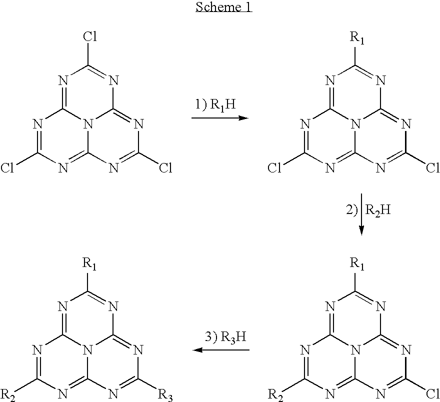

Heptaazaphenalene derivatives of general formula (I), wherein R₁, R₂ and R₃ are the same and represent a —NR₅R₆ radical, wherein R₅ and R₆ have the meaning as defined above, can be obtained by reacting the 2,5,8-trichloro-1,3,4,6,7,9,9b-heptaazaphenalene derivative of formula (IV) with a derivative of general formula (V)

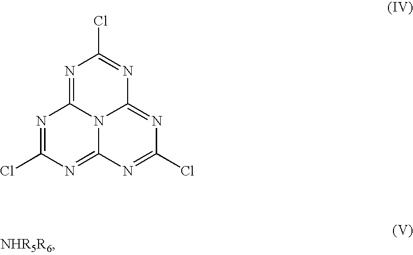

in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent, optionally in the presence of an organic base comprising diisopropylethylamine, triethylamine or pyridine, or an inorganic base comprising potassium carbonate, sodium hydroxide, sodium carbonate, cesium carbonate or sodium bicarbonate.

Any of the steps described in the above procedures can also be carried out in a microwave oven, employing a typical procedure of MAOS (microwave assisted organic synthesis). This standard method is well known by those skilled in the art.

Common microwave equipment may be used in preparation processes according to the present invention. The microwave irradiation may be performed at a power level of 1 to 1600 W, preferably 1 to 300 W, and particularly preferably about 70 W. The duration for the microwave irradiation may vary according to conditions such as the amount or reactant but may be in the range from 20 seconds to 60 minutes, preferably from 1 minute to 20 minutes. The reaction can be carried out at a temperature of 50-280° C., preferably 80-200° C., and more preferably 120-150° C., with of without solvent, under microwave irradiation. A presently preferred microwave furnace is commercially available from CEM, Inc., as model Discover®. The Discover® System incorporates temperature and pressure feedback systems, for example, an infrared temperature sensor positioned below the reaction vessel, for complete control of the reaction. As described above, according to the present invention, heptaazaphenalene derivatives can be prepared within a very short time, i.e. several seconds to several minutes, by microwave irradiation, unlike conventional techniques requiring about 12-50 hours for preparation of compounds for general formula I.

Heptaazaphenalene derivatives of general formula (I), wherein R₁, R₂ and R₃ are the same and represent a —NR₅R₆ radical, wherein R₅ and R₆ have the meaning as defined above and wherein one of the radicals R₁, R₂ and R₃ is different from the other two of these radicals R₁, R₂ and R₃ can be obtained by

a) reacting a 2,5,8-trichloro-1,3,4,6,7,9,9b-heptaazaphenalene derivative of formula (IV) react with a derivative of general formula (V)

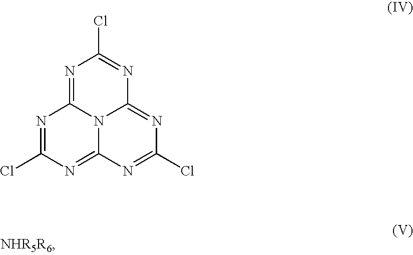

wherein R₅ and R₆ have the meaning as defined above, in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent; optionally in the presence of an organic base comprising diisopropylethylamine, triethylamine or pyridine, or an inorganic base comprising potassium carbonate, sodium hydroxide, sodium carbonate, cesium carbonate or sodium bicarbonate; and
b) adding to the mixture resulting from the preceding stage a second derivative of general formula (V) different from the one used in stage (a) and submitting to reflux. Any of the steps described above can be conducted trough MAOS (microwave assisted organic synthesis).

Heptaazaphenalene derivatives of general formula (I), wherein R₁, R₂ and R₃ are different from each other and a —NR₅R₆ radical, wherein R₅ and R₆ have the meaning as defined above, can be obtained by

a) reacting the 2,5,8-trichloro-1,3,4,6,7,9,9b-heptaazaphenalene derivative of formula (IV) with a derivative of general formula (V)

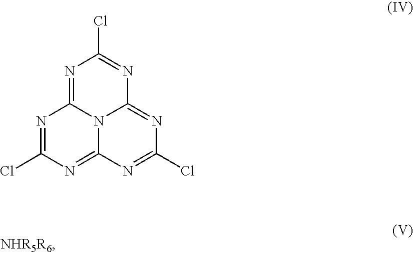

wherein R₅ and R₆ have the meaning as defined above, in a solvent comprising 1,4-dioxane, tetrahydrofuran, toluene, tetrahydrofuran, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent and more preferably between 50° C. and the boiling temperature of the solvent; optionally in the presence of an organic base comprising diisopropylethylamine, triethylamine or pyridine, or an inorganic base comprising potassium carbonate, sodium hydroxide, sodium carbonate, cesium carbonate or sodium bicarbonate;
b) adding to the resulting mixture a derivative of general formula (V) different from the one used in the preceding stage

NHR₅R₆ (V)

wherein one of R₅ and R₆ have the meaning as defined above, and submitting to reflux; and
c) adding to the mixture resulting from stage (b) a derivative of general formula (V) different from the one used in stages (a) and (b)

NHR₅R₆ (V)

wherein R₅ and R₆ have the meaning as defined above.

Any of the steps described above can be conducted trough MAOS (microwave assisted organic synthesis).

Heptaazaphenalene derivatives of general formula (I), wherein R₁, R₂ and R₃ are the same and represent a derivative of general formula (III)

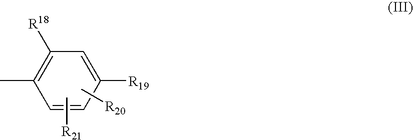

can be obtained by reacting 2,5,8-trichloro-1,3,4,6,7,9,9b-heptaazaphenalene derivative of formula (IV) with a heterocyclic derivative or a compound of general formula (VI)

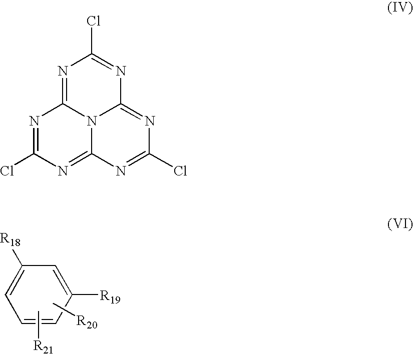

wherein R₁₈, R₁₉, R₂₀ and R₂₁ have the meaning as defined above, in the presence of a Lewis acid comprising, FeCl₃, BF₃, in particular aluminium trichloride, in an inert solvent comprising toluene, 1,1,2,2-tetrachloroethane, tetrahydrofuran, 1,2-dichlorobenzene, nitrobenzene or benzene and at a temperature that ranges between 60° C. and the boiling temperature of the solvent.

Any of the steps described above can be conducted trough MAOS (microwave assisted organic synthesis).

Heptaazaphenalene derivatives of general formula (I), wherein one of the radicals R₁, R₂ and R₃ represents an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and

the other two radicals of the radicals R₁, R₂ and R₃ are the same and represent a —NR₅R₆ radical, wherein R₅ or R₆ have the meaning as defined above, can be obtained by reacting a derivative of general formula (VII) with a derivative of general formula (V):

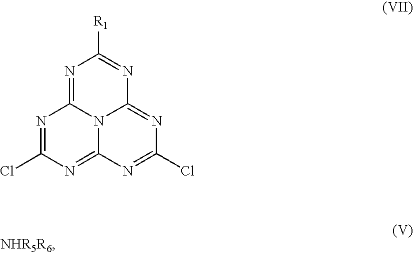

wherein R₁ represents an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and

R₅ and R₆ have the meaning as defined above,

in an inert solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent.

Any of the steps described above can be conducted trough MAOS (microwave assisted organic synthesis).

The derivative of general formula (VII) depicted above can be obtained by reaction of the 2,5,8-trichloro-1,3,4,6,7,9,9b-heptaazaphenalene derivative of general formula (V) with an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S, in an inert solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent and more preferably between 50° C. and the boiling temperature of the solvent.

Any of the steps described above can be conducted trough MAOS (microwave assisted organic synthesis).

Heptaazaphenalene derivatives of general formula (I), wherein one of the radicals R₁, R₂ and R₃ are the same and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and

the third of the radicals R₁, R₂ and R₃ represents a —NR₅R₆ radical, wherein R₅ and R₆ are defined as defined above, can be obtained by reacting the 2,5,8-trichloro-1,3,4,6,7,9,9b-heptaazaphenalene derivative of formula (IV) with a derivative of general formula (V)

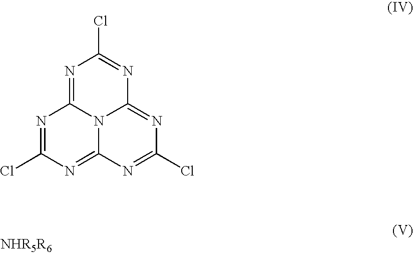

wherein R₅ and R₆ have the meaning as defined above, in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent, optionally in the presence of an organic base such as diisopropylethylamine, triethylamine or pyridine, or an inorganic base comprising potassium carbonate, sodium hydroxide, sodium carbonate, cesium carbonate or sodium bicarbonate to obtain a compound of general formula (VIII),

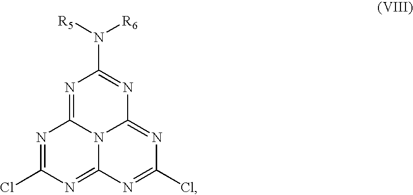

wherein R₅ and R₆ have the meaning as defined above, and wherein said derivative of general formula (VIII) is reacted with a compound of general formula (VI)

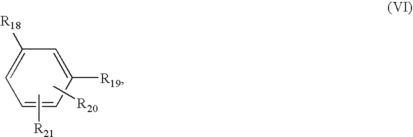

wherein R¹⁸, R¹⁹, R²⁰ and R²¹ have the meaning as defined above, in the presence of a Lewis acid comprising, FeCl₃, BF₃, in particular aluminium chloride, in an inert solvent comprising toluene, xylene, 1,1,2,2-tetrachloroethane, tetrahydrofurane, 1,2-dichlorobenzene, nitrobenzene or benzene at a temperature between 60° C. and the boiling temperature of the solvent.

Any of the steps described above can be conducted trough MAOS (microwave assisted organic synthesis).

The compounds of general formula (I), wherein R₂₀ represents a —SO₃M radical, wherein M has the meaning as defined above, can be obtained by carrying out, for example, the methods disclosed in U.S. Pat. No. 6,090,370, in particular column 5, line 59-column 6, line 8.

The compounds of general formula (I), wherein an —SO₃M group, with M having the meaning as defined above, has been introduced into an alkylic chain, can be obtained according to the methods described in Lewin, G. et al., J. Nat. Prod., 58 (1995) 12, 1840-1847.

The compounds of general formula (I), wherein an —N(R₁₁)₃⁺ group, with R₁₁ having the meaning as defined above, has been inserted into an alkylic chain, can be obtained for example by following the methods described in Sharma, M. L. et al., J. Indian Chem. Soc., 74 (1997)₄, 343-344.

The combination of active substances (A) and (B) according to the present invention has preferably an UV-A/UV-B protection ratio in the range of 0.1 to 1, more preferably 0.2 to 1, most preferably 0.3 to 1.

The UV-A/UV-B protection ratio defines the performance of a sunscreen in the UV-A radiation range (320-400 nm) in relation to its performance in the UV-B radiation range (290-320 nm). It is calculated as the ratio between the areas defined by the UV-A and UV-B radiation absorption capacity. A ratio value of 1 indicates that a sunscreen absorbs equally in UV-A and UV-B radiation range, but is silent about the absolute absorption capacity of a sunscreen. Preferred UV-A/UV-B protection ratios for the inventive combination are in the range as mentioned before.

For the efficiency and/or the suitability of a sunscreen it is convenient to also know its absolute absorption capacity.

The inventive combination absorbs preferably at least 20, 30, 40, 50, 60, 65, 70, 75, 80, 85, 90 or 95% of the total UV-A and UV-B radiation (290 to 400 nm), more preferably at least 50%, even more preferably at least 70%, most preferably at least 85% of the total UV-A and UV-B radiation range (290 to 400 nm).

The term “absorption of the total UV-A and UV-B radiation” according to the present invention is a measure for the absorption capacity of the inventive combination and refers to the overall absorption in a radiation range from 290 to 400 nm. The total absorption capacity is given by the area under the absorption curve (AUC) in the UV radiation range from 290 to 400 nm.

Preferably, the inventive combination absorbs not only at least 10, 20, 30, 40, 50, 60, 65, 70, 75, 80, 85, 90 or 95% of the total UV-A radiation (320 to 400 nm), but also at least 10, 20, 30, 40, 50, 60, 65, 70, 75, 80, 85, 90 or 95% of the total UV-B radiation (290 to 320 nm). In case that the inventive combination absorbs in both the UV-A and UV-B radiation range, the absorption of the total UV-A and UV-B radiation must not exceed 100%. For example, if the inventive combination absorbs 20% in the UV-A radiation range, the maximal absorption in the UV-B radiation range may be 80%.

Another aspect of the present invention is a pharmaceutical composition comprising the combination of UV-absorbing substances (A) and (B) as defined above and optionally at least one pharmacologically acceptable auxiliary agent, or a cosmetical composition comprising the combination of UV-absorbing substances (A) and (B) as defined above and optionally at least one cosmetical vehicle.

According to the present invention, the inventive pharmaceutical composition comprising the combination of UV-absorbing substances (A) and (B) as defined above may contain at least one further pharmaceutically active substance. Such further pharmaceutically active substance is preferably suitable for the prophylaxis or the treatment of diseases caused by and/or associated with exposure to UV radiation to the skin of mammals, preferably human beings, in order to achieve a sufficient therapeutical effect.

The amount of the UV-absorbing active substance (A) and of the UV-absorbing active substance (B) in an inventive pharmaceutical or cosmetical composition is preferably in the range of 0.01% to 30% per weight, more preferably 0.01% to 20% per weight, most preferably 0.1% to 15% per weight, based on the total weight of the composition.

In the inventive pharmaceutical or cosmetical composition at least one UV-absorbing active substance (A) or (B) may be present in micronized form, having an average particle size preferably in the range of 0.001 to 4 μm, more preferably in the range of 0.005 to 2 μm, most preferably in the range of 0.01 to 1 μm.

At least one of the following auxiliary agents can be present in the inventive pharmaceutical as well as in the inventive cosmetical composition: gelling agents, oils, waxes, thickening agents, hydrophilic or hydrophobic polymers, emulsifying agents, emollients, fatty acids, organic solvents, antioxidants, stabilizers, sequestering agents, acidifying or basifying agents, emulsifiers, emollients, surfactants, film formers, biological additives to enhance performance and/or consumer appeal such as amino acids, proteins, vanilla, aloe extract or bioflavinoids, buffering agents, chelating agents such as ethylenediaminetetra-acetic acid (EDTA) or oxalic acid, colorants, dyes, propellants, antifoaming agents, wetting agents, vitamins, emulsion stabilizers, pH adjusters, thickening agents, fragrances, preservatives, opacifying agents, water and/or alcohols. The aforementioned auxiliary agents for the inventive compositions are used in the usual amounts known by those skilled in the art.

As oils for the inventive compositions oils from animal or vegetable sources or synthetic are preferably used. Particularly preferred are oils selected from the group comprising liquid petrolatum, liquid paraffin, volatile and non-volatile silicone oils, isoparaffins, polyalphaolefins, fluorated and perfluorated oils.

As stabilizers for the inventive compositions preferably non-ionic, anionic, cationic and amphiphilic tensides can be used, which are preferably selected from the group comprising polyethylenglycol (PEG) and derivatives thereof, tweens, tritons, spans, polygycerines, polyalkyl glycerides, alkyl sulfonates, aryl sulfonates, alkyl phosphates, derivatives of alkyl-betaine and phosphatidylglycerole.

Emulsifiers are preferably used in certain formulations of the inventive compositions in amounts effective to provide uniform blending of ingredients of the composition. Useful emulsifiers include anionics such as fatty acid soaps, e.g., potassium stearate, sodium stearate, ammonium stearate, and triethanolamine stearate; polyol fatty acid monoesters containing fatty acid soaps, e.g., glycerol monostearate containing either potassium or sodium salt; sulfuric esters (sodium salts), e.g., sodium lauryl 5 sulfate, and sodium acetyl sulfate; and polyol fatty acid monoesters containing sulfuric esters, e.g., glyceryl monostearate containing sodium lauryl surfate; (ii) cationics chloride such as N(stearoyl colamino formylmethyl)pyridium; N-soya-N-ethyl morpholinium ethosulfate; alkyl dimethyl benzyl ammonium chloride; diisobutylphenoxytheoxyethyl dimethyl benzyl ammonium chloride; and acetyl pyridium chloride; and (iii) nonionics such as polyoxyethylene fatty alcohol ethers, e.g., monostearate; polyoxyethylene lauryl alcohol; polyoxypropylene fatty alcohol ethers, e.g., propoxylated oleyl alcohol; polyoxyethylene fatty acid esters, e.g., polyoxyethylene stearate; polyoxyethylene sorbitan fatty acid esters, e.g., polyoxyethylene sorbitan monostearate; sorbitan fatty acid esters, e.g., sorbitan; polyoxyethylene glycol fatty acid esters, e.g., polyoxyethylene glycol monostearate; and polyol fatty acid esters, e.g., glyceryl monostearate and propylene glycol monostearate; and ethoxylated lanolin derivatives, e.g., ethoxylated lanolins, ethoxylated lanolin alcohols and/or ethoxylated cholesterol.

Emollients may be used in the formulations of the inventive compositions in such amounts to prevent or relieve dryness. Useful emollients include, without limitation hydrocarbon oils and waxes; silicone oils; triglyceride esters; acetoglyceride esters; ethoxylated glyceride; alkyl esters; alkenyl esters; fatty acids; fatty alcohols; fatty alcohol ethers; etheresters; lanolin and derivatives; polyhydric alcohols (polyols) and polyether derivatives; polyhydric alcohol (polyol) esters; wax esters; beeswax derivatives; vegetable waxes; phospholipids; sterols; and/or amides.

Surfactants can be used in certain formulations of the inventive compositions. Suitable surfactants are for example those surfactants generally grouped as cleansing agents, emulsifying agents, foam boosters, hydrotropes, solubilizing agents, suspending agents and non-surfactants, which facilitate the dispersion of solids in liquids.

Suitable film formers which are preferably used in the formulations of the inventive compositions should keep the composition smooth and even and are preferably, without limitation, at least one substance selected from the group comprising acrylamide/sodium acrylate copolymer; ammonium acrylates copolymer; Balsam Peru; cellulose gum; ethylene/maleic anhydride copolymer; hydroxyethylcellulose; hydroxypropylcellulose; polyacrylamide; polyethylene; polyvinyl alcohol; pvm/MA copolymer (vinyl methylether/maleic anhydride copolymer); PVP (polyvinylpyrrolidone); maleic anhydride polymer, vinylpyrrolidon/hexadecene copolymer; acryliclacrylate copolymer and the like.

pH adjusters may also be used in certain formulations of the inventive compositions. These pH adjusters preferably comprise, but are not limited to ammonium hydroxide, triethanolamine or citric acid.

Thickening agents used for the formulations of the inventive compositions preferably are, but are not limited to candelilla, carnauba, and microcrystalline waxes, crosslinked acrylic-acid polymers, carbomer, methylhydroxyethylcellulose, hydroxypropylmethylcellulose or hydroxyethylcellulose. and polyethylene thickeners.

Examples of preferred organic solvents for the inventive compositions include lower aliphatic alcohols and polyols.

Suitable antioxidants suitable for the inventive compositions are preferably selected from the group comprising ascorbic acid (vitamin C), sodium-L-ascorbate, calcium-L-ascorbate, ascorbyl palmitate, butylhydroxyanisole, butylhydroxytoluene, calcium-disodium-EDTA, isoascorbic acid, lecitine, lactic acid, polyphosphate, tocopherol (vitamin E), like α-tocopherol, γ-tocopherol, δ-tocopherol, propylgallate, octylgallate, dodecylgallate, sodium-isoascorbate, citric acid, sodium citrate, potassium citrate and tin-II-chloride.

Gelling agents, which are preferably used in the formulations of the inventive compositions, can be natural or synthetic polymers. Natural polymers are preferably selected from the group comprising Agar-Agar, alginate, pectin, carbomer, carrageenan, casein, dextrine, gelatine, arabic gum, keratine, locust bean gum, xanthan gum and the like. Preferred synthetic polymers which can be used in the formulations of the inventive compositions are selected from the group comprising acylic acid polymers, polyacryl amides and alkylene oxide polymers.

For the inventive cosmetical composition marketed in Europe the respective components shall be used in amounts which are in compliance with the Council Directives 76/768/EEC and Commission Directive 95/17/EC on the approximation of the laws of the Member States relating to cosmetic products.

The inventive pharmaceutical or cosmetical composition is preferably adapted at least for a once a day, twice or three, four or five times a day, preferably for twice a day application.

For the application, preferably topical application, the inventive pharmaceutical or cosmetical composition may be formulated in liquid or in semi-solid form, preferably as liquid, fluid, foam, cream, gel, paste, balsam, spray, ointment, lotion, conditioner, tonic, milk, mousse, emulsion, serum, oil, stick, shampoo, jelly, suspension, dispersion, lacquer, paint, elixir, drop or aerosol.

Preferably the inventive pharmaceutical or cosmetical compositions are suitable for protecting the skin against UV radiation, either in the range of UV-A or UV-B or the total UV-A and UV-B range.

A measure for the UV-protecting property of an UV-absorbing composition is the “sun protection factor” (SPF). The SPF of an UV-absorbing composition determined in vivo is a universal indicator of the efficacy against sunburn—the higher the SPF, the more effective is the protection against UV radiation. The level of sun protection has traditionally been estimated using the sun protection factor or SPF test, which utilises the erythemal response of the skin to ultraviolet (UV) radiation. The SPF is a ratio calculated from the energies required to induce a minimum erythemal response with and without sun product applied to the skin of human volunteers, using ultraviolet radiation usually from an artificial source. The determination of the SPF is described in detail in the following references, which are herewith introduced as reference and shall form part of the disclosure or the present application: Diffey B L et., “In vitro assessment of the broad spectrum ultraviolet protection of sunscreen products”, J. Am. Acad. Dermatol., 2000, 43(6), 1024-1035 respectively Diffey B. “A method for broad-spectrum classification on sunscreens”, Int J Cosmet Sci. 1994; 16:47-52.

Preferably, the SPF of ane inventive pharmaceutical or cosmetical compositions is at least 8, more preferably at least 10, even more preferably at least 15 and most preferably at least 20.

The UV-A/UV-B ration of an inventive pharmaceutical or cosmetical compositions can be also determined according to the methods disclosed in the before mentioned publications of Diffey B. respectively Diffey B L.

The inventive pharmaceutical or cosmetical compositions are particularly suitable for keeping scalp, skin, hair and/or nails in good conditions or for moisturizing the skin, treating dry skin successfully and/or against skin ageing and/or photoageing.

Moreover, the inventive combinations are preferably suitable for the prophylaxis and/or treatment of diseases caused by and/or associated with exposure to ultraviolet radiation on the skin, preferably the lips, the face and/or the body of mammals.

Human exposure to solar ultraviolet radiation has important public health implications. Evidence of harm associated with overexposure to UV radiation has been demonstrated in many studies (see e.g. Environmental Burden of Disease Series, No. 13, 2006; Solar Ultraviolet Radiation—Global burden of disease from solar ultraviolet radiation, World Health Organization). Skin cancer and malignant melanoma are among the most severe health effects, but a series of other health effects, such as polymorphous light eruption, actinic keratosis, solar urticaria, xeroderma pigmentosum, photoageing, chronic actinic dermatitis or sunburn have been identified.

Skin cancer is an increasingly common condition, in part attributed to increased exposure to ultraviolet radiation. The increased exposure is mainly due to the recent popularity of sun tanning (sun bathing). Lighter-skinned individuals are more vulnerable. In the United States, about one out of every three new cancers arises from skin. The most common types are basal cell carcinoma (BCC) and squamous cell carcinoma (SCC) which may be locally disfiguring but unlikely to spread to other parts of the body. The most dangerous type is malignant melanoma, which can be fatal if not treated early, but forms only a small proportion of all skin cancers.

Squamous cell carcinoma is a form of cancer of the carcinoma type that may occur in many different organs, including the skin, mouth, esophagus, lungs, and cervix. It is a malignant tumour of epithelium that shows squamous cell differentiation. Squamous cell carcinomas account for about 20% of non-melanoma skin cancers, (with basal cell carcinomas accounting for about 80%), but are clinically more significant because of their ability to metastasize. Squamous cell carcinoma is usually developed in the epithelial layer of the skin and sometimes in various mucous membranes of the body. This type of cancer can be seen on the skin, lips, inside the mouth, throat or esophagus. This type of cancer is characterized by red, scaly skin that becomes an open sore. One risk factor for squamous cell carcinoma is exposure to sunlight.

Basal cell carcinoma (BCC) is the most common skin cancer. It can be destructive and disfiguring. Risk is increased for individuals with a family history of the disease and a high cumulative exposure to UV light via sunlight or, in the past, carcinogenic chemicals especially arsenic. BCC is much more common in fair skinned individuals with a family history of basal cell cancer and increases in incidence closer to the equator at higher altitude. Most sporadic BCC arise in small numbers on sun-exposed skin of people over age 50, although younger people may also be affected.

Polymorphic light eruption is a common rash that occurs as a result of photosensitivity. Polymorphic light eruption (PMLE) generally occurs in adult females aged 20 to 40, although it sometimes affects children and rarely males. It is more common in places where sun exposure is uncommon, such as Northern Europe, where it is said to affect 10% of women holidaying in the Mediterranean. It can be the first sign of lupus erythematosus, but this is not usually the case. The name ‘polymorphic’, or ‘polymorphous’ refers to the fact that the rash can take many forms, although in one individual it usually looks the same every time it appears. The commonest variety is crops of 2-5 mm pink or red raised spots occurring on the arms. Other areas may be involved, particularly the chest and lower legs, but the face is usually spared.

Actinic keratosis is one type of skin cancer, which appears as rough, red or brown, scaly patches on the skin. It is a precancerous condition and develops sometimes into squamous cell cancer.

Solar urticaria is the development of hives minutes after exposure to the sun, which resolve in less than 1 hour. Tan skin that has been previously exposed may not react. Solar urticaria is divided into 6 different types depending on the wavelength of light involved.

Xeroderma pigmentosum, or XP, is a genetic disorder of DNA repair in which the body's normal ability to remove damage caused by ultraviolet (UV) light is deficient. This leads to multiple basaliomas and other skin malignancies at a young age. In severe cases, it is necessary to avoid sunlight.

Skin ageing is a normal process of life but one that many individuals do not take gracefully. With increasing age, cellular cohesion is reduced, renewal time is slower and the elastic tissue degenerates resulting in wrinkles and sagging. Degenerative changes in elastic tissue begin at about 30. The rate of age related changes is influenced by heredity, personal hygiene practices, nutrition, general health and history of sun, radiation and chemical exposure. During the ageing process, cellular cohesion is reduced, the renewal time of the cell layer slows, the moisture content is diminished. The elastic tissues is degraded by enzymes. Photoageing is characterised by chronic inflammation, elastosis.

Chronic actinic dermatitis (CAD), sometimes known as photosensitivity dermatitis/actinic reticuloid syndrome (PD/AR), is a condition in which the skin becomes inflamed, particularly in areas that have been exposed to sunlight or artificial light.

A sunburn is a burn to the skin produced by overexposure to ultraviolet (UV) radiation, commonly from the sun's rays. A similar burn can be produced by overexposure to other sources of UV such as from tanning lamps, or occupationally, such as from welding arcs. Exposure of the skin to lesser amounts of UV will often produce a suntan. Usual mild symptoms are red or reddish skin that's hot to the touch, a washed out feeling, and mild dizziness. Sunburn can be life-threatening and is a leading cause of cancer.

Thus, a further aspect of the present invention relates to the use of the inventive combination mentioned above for the preparation of a pharmaceutical composition for the prophylaxis and/or treatment of diseases caused by and/or associated with exposure to ultraviolet radiation, preferably of the lips, the face and/or of the body of mammals, preferably human beings, more preferably polymorphous light eruption, actinic keratosis, solar urticaria, xeroderma pigmentosum, photoageing, chronic actinic dermatitis and/or sunburn.

Another preferred aspect is the use of the inventive cosmetical composition for the prophylaxis and/or care against non-desired skin conditions comprising polymorphous light eruption, actinic keratosis, solar urticaria, xeroderma pigmentosum, photoageing chronic actinic dermatitis and/or sunburn.

Another preferred aspect is the use of the inventive cosmetical composition for the prophylaxis and/or care against non-desired skin conditions comprising polymorphous light eruption, actinic keratosis, solar urticaria, xeroderma pigmentosum, photoageing chronic actinic dermatitis and/or sunburn.

Yet, another preferred embodiment of the present invention is the use of the inventive cosmetical composition for protecting the skin preferably the lips, the face and/or the body of a mammal, preferably a human, against ultraviolet radiation, preferably UV-A and/or UV-B radiation.

### EXAMPLES

In all examples given below the % by weight of the respective components are based on the total weight of the composition.

The inventive formulations according to the examples are prepared by standard techniques known by those skilled in the art.

### Example 1

| A cream formulation with the composition: | | % by weight |
|---|---|---|
| A) | 2,5,8-tris-(4-(imidazo[1,2-a]pyridin-2-yl)-phenylamino)-1,3,4,6,7,9,9b- | 3% |
| | heptaazaphenalene [λₘₐₓ = 353 nm] | |

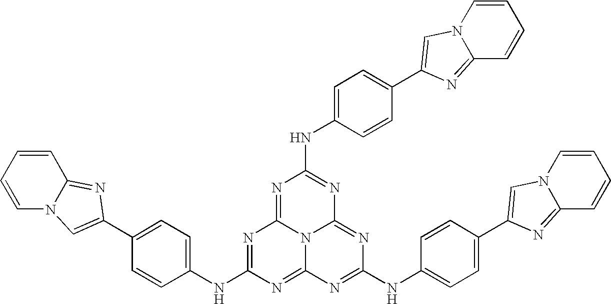

| | | |
|---|---|---|
| B) | 2-hydroxy-4-methoxy benzophenone (Benzophenon-3) [λₘₐₓ = 288 nm] | 5% |
| C) | stearyl alcohol (Nacol 18-98 (Sasol)) | 1.2% |
| D) | PEG-40 stearate (Simulsol M52 (Seppic)) | 2.5% |
| E) | cetearyl ehtylhexanoate (DUB Liquide 85) | 2.5% |
| F) | caprylic/capryc triglyceride (Miglyol 812 N (Sasol) | 3.0% |
| G) | C₁₂₋₁₅ alkyl benzoate (Tegosoft TN (Goldschmidt)) | 5.0% |
| H) | phenoxyethanol (and) methylparaben (and) ethylparaben | 0.6% |
| | (and) propylparaben (and) isobutylparaben (and) | |
| | butylparaben (Phenonip (Clariant)) | |
| I) | polyacrylamide (and) C₁₃₋₁₄ isoparaffin (and) | 2% |
| | Laureth-7 (Sepigel 305 (Seppic)) | |
| J) | cellulose gum (Blanose Cellulose Gum 7H3SF (Hercules)) | 0.25% |
| K) | water | ad 100% |

is prepared.

### Example 2

| | | % |
|---|---|---|
| A cream formulation with the composition: | | by weight |
| A) | 2,5,8-tris-(4-benzoylphenylamino)-1,3,4,6,7,9,9b-hepta- | 3% |
| | azaphenalene [λₘₐₓ = 339 nm] | |

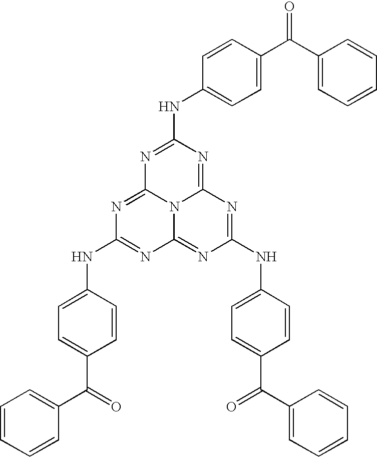

| | | |
|---|---|---|
| B) | ethylhexyl methoxycinnamate (Parsol MCX) | 10% |
| | [λₘₐₓ = 310 nm] | |
| C) | butyl methoxydibenzoylmethane | 3% |
| | (Parsol 1789, Avobenzone) [λₘₐₓ = 357 nm] | |
| D) | stearyl slcohol (Nacol 18-98 (Sasol)) | 1.2% |
| E) | PEG-40 stearate (Simulsol M52 (Seppic)) | 2.5% |
| F) | cetearyl ethylhexanoate (DUB Liquide 85) | 2.5% |
| G) | caprylic/capryc triglyceride (Miglyol 812 N (Sasol)) | 3.0% |
| H) | C₁₂₋₁₅ alkyl benzoate (Tegosoft TN (Goldschmidt)) | 5.0% |
| I) | phenoxyethanol (and) methylparaben (and) ethylparaben | 0.6% |
| | (and) propylparaben (and) isobutylparaben (and) | |
| | butylparaben (Phenonip (Clariant)) | |
| J) | polyacrylamide (and) C₁₃₋₁₄ isoparaffin (and) | 2% |
| | Laureth-7 (Sepigel 305 (Seppic)) | |
| K) | cellulose gum (Blanose Cellulose Gum 7H3SF | 0.25% |
| | (Hercules)) | |
| L) | water | ad 100% |

is prepared.

### Example 3

| A cream formulation with the composition: | | % by weight |
|---|---|---|
| A) | 2,5,8-tris-(4-((E)-3-ethoxy-3-oxoprop-1-enyl)phenylamino)-1,3,4,6,7,9,9b- | 3% |
| | heptaazaphenalene [λₘₐₓ = 359 nm] | |

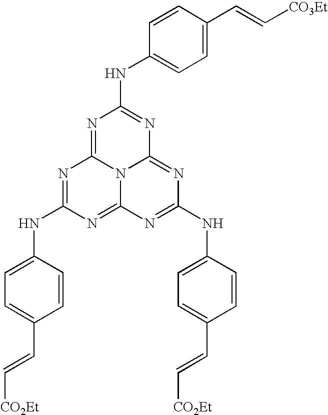

| | | |
|---|---|---|
| B) | ethylhexyl methoxycinnamate (Parsol MCX) [λₘₐₓ = 310 nm] | 5% |
| C) | methylene bis-benzotrianolyl tetramethylbutylphenol (and) | 5% |
| | decylglucoside (and) propylene glycol (and) | |
| | xanthan gum (Tinosorb M (Ciba)) [λₘₐₓ = 358 nm] | |
| D) | stearyl alcohol (Nacol 18-98 (Sasol)) | 1.2% |
| E) | PET-40 stearate (Simulsol M52 (Seppic)) | 2.5% |
| F) | cetearyl ethylhexanoate (DUB Liquide 85) | 2.5% |
| G) | caprylic/capryc triglyceride (Miglyol 812 N (Sasol)) | 3.0% |
| H) | Dimethicone (D 200 Fluid 350 CST (Dow Corning)) | 0.5% |
| I) | phenoxyethanol (and) methylparaben (and) ethylparaben | 0.6% |
| | (and) propylparaben (and) isobutylparaben (and) | |
| | butylparaben (Phenonip (Clariant)) | |
| J) | polyacrylamide (and) C₁₃₋₁₄ isoparaffin (and) | 2% |
| | Laureth-7 (Sepigel 305 (Seppic)) | |
| K) | cellulose gum (Blanose Cellulose Gum 7H3SF (Hercules)) | 0.25% |
| L) | water | ad 100% |

is prepared.

### Example 4

| A cream formulation with the composition: | | % by weight |
|---|---|---|
| A) | 2,5,8-tris-(2-ethylhexylamino)-1,3,4,6,7,9,9b- | 3% |
| | heptaazaphenalene [λₘₐₓ = 265 nm] | |

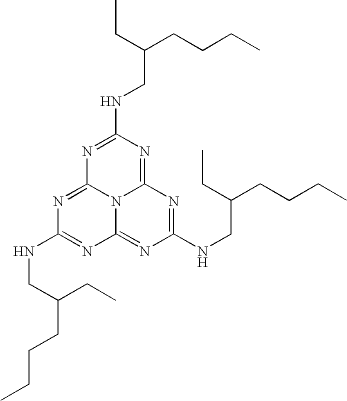

| | | |
|---|---|---|
| B) | Tinosorb M [λₘₐₓ = 358 nm] | 6% |
| C) | stearyl alcohol (Nacol 18-98 (Sasol)) | 1.2% |
| D) | PEG-40 stearate (Simulsol M52 (Seppic)) | 2.5% |
| E) | cetearyl ethylhexanoate (DUB Liquide 85) | 2.5% |
| F) | caprylic/capryc triglyceride (Miglyol 812 N (Sasol)) | 3.0% |
| G) | C₁₂₋₁₅ alkyl benzoate (Tegosoft TN (Goldschmidt)) | 5.0% |
| H) | phenoxyethanol (and) methylparaben (and) ethyl- | 0.6% |
| | paraben (and) propylparaben (and) isobutylparaben | |
| | (and) butylparaben (Phenonip (Clariant)) | |
| I) | polyacrylamide (and) C₁₃₋₁₄ isoparaffin (and) | 2% |
| | Laureth-7 (Sepigel 305 (Seppic)) | |
| J) | cellulose gum (Blanose Cellulose Gum 7H3SF | 0.25% |
| | (Hercules)) | |
| K) | water | ad 100% |

is prepared.

1. A combination of at least 2 UV-absorbing active substances (A) and (B), characterized in that
the UV-absorbing active substance (A) is at least one heptaazaphenalene compound of the general formula (I), 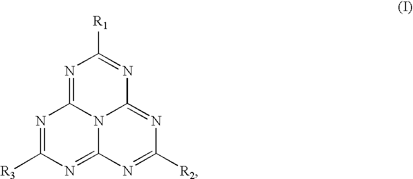
wherein
R₁, R₂ and R₃, independent of each other, each represents an unsubstituted or at least mono-substituted mono- or polycyclic aryl radical; an unsubstituted or at least mono-substituted, saturated, unsaturated or aromatic heterocyclic radical having from 5 to 10 atoms that can contain 1, 2 or 3 heteroatoms independently selected from O, N and S; an —OR₄ radical; or a —NR₅R₆ radical;
R₄ represents a hydrogen atom; an unsubstituted or at least mono-substituted, saturated or unsaturated, linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted, saturated, unsaturated or aromatic heterocyclic radical having from 5 to 14 atoms that can contain 1, 2 or 3 heteroatoms independently selected from O, N and S;
R₅ and R₆, independent of each other, each represents a hydrogen atom; an unsubstituted or at least mono-substituted, saturated or unsaturated, linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted, saturated, unsaturated or aromatic heterocyclic radical having from 5 to 10 atoms that can contain 1, 2 or 3 heteroatoms independently selected from O, N and S;
or
R₅ and R₆ form together with the bridging nitrogen atom an unsubstituted or at least mono-substituted saturated, unsaturated or aromatic mono- or polycyclic ring system having from 4 to 10 atoms that contains optionally 1 or 2 heteroatoms independently selected from N, O and S;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} either in the range of UV-A or UV-B radiation
and
the UV-absorbing active substance (B) is at least one compound, which has its absorption maximum λ_{max(B)} either in the range of UV-A or UV-B radiation, preferably the λ_{max(B)} of active substance (B) does not lie in the same UV radiation range wherein the λ_{max(A)} of active substance (A) lies, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1, preferably from 1:10 to 20:1;
with the exception of the combination of 37.5% by weight, based on the weight of the combination, of 2,5,8-tris-(4-(butoxycarbonyl)phenylamino)-1,3,4,6,7,9,9b-heptaazaphenalene 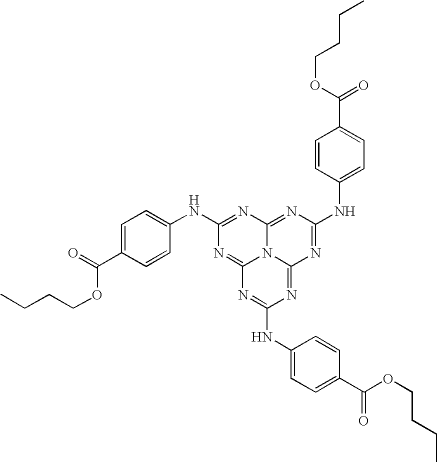
and 62.5% by weight, based on the weight of the combination, of ethylhexyl methoxycinnamate;
with the exception of the combination of 60.5 to 67.6% by weight, based on the weight of the combination, of 2-(4-(carboxy)phenylamino)-5,8-bis-(4-methylphenyl)-1,3,4,6,7,9,9b-heptaazaphenalene 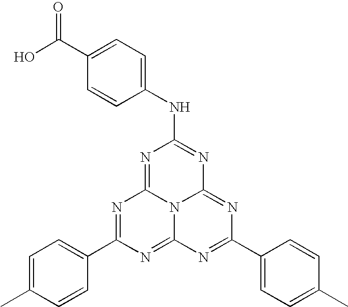
and 32.4 to 39.5% by weight, based on the weight of the combination, of titanium dioxide,
and
with the exception of the combination of 60.5 to 67.6% by weight, based on the weight of the combination, of 2,5,8-tris-(4-(2-ethylhexyloxycarbonyl)phenylamino)-1,3,4,6,7,9,9b-heptaazaphenalene 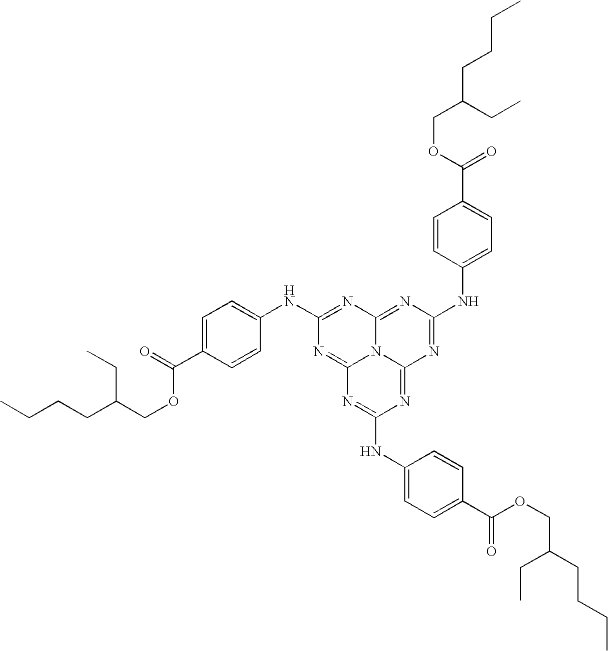
and 32.4 to 39.5% by weight, based on the weight of the combination, of titanium dioxide.

2. A combination according to claim 1, characterized in that the UV-absorbing active substance (A) is at least one heptaazaphenalene compound of general formulae (IA) to (IL) 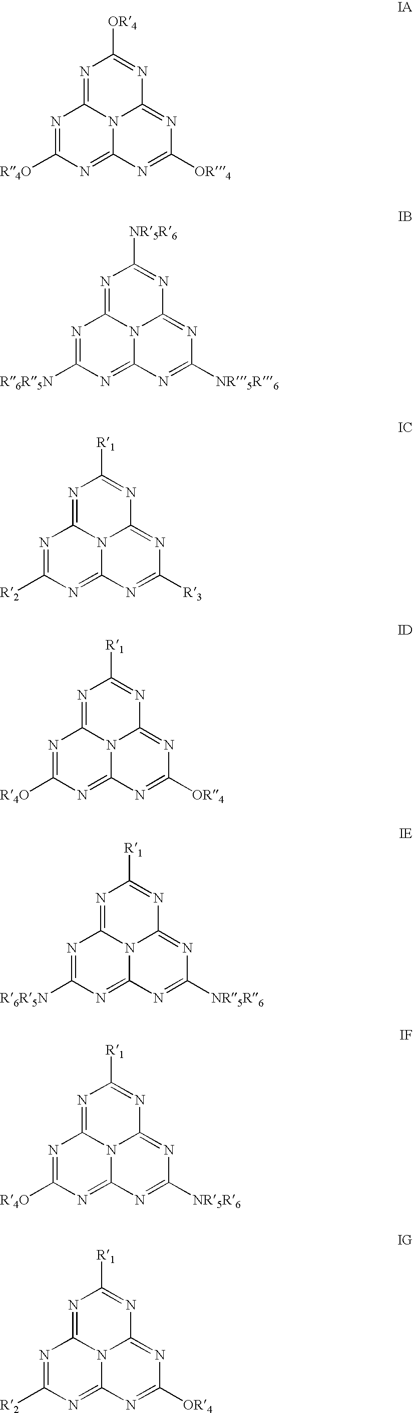 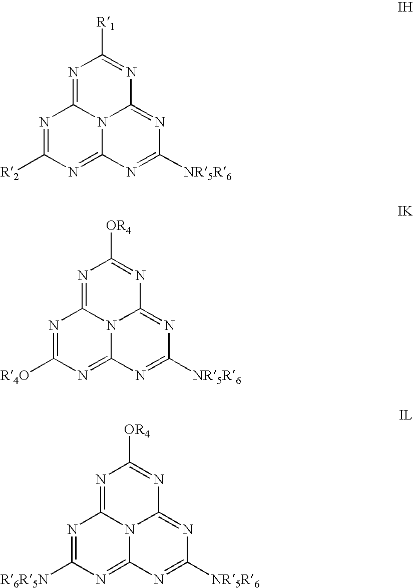
wherein
R′₁, R′₂ and R′₃, independent of each other, each represents an unsubstituted or at least mono-substituted, mono- or polycyclic aryl radical; an unsubstituted or at least mono-substituted, saturated, unsaturated or aromatic heterocyclic radical having from 5 to 10 atoms that can contain 1, 2 or 3 heteroatoms independently selected from O, N and S;
R₄, R′₄, R″₄ and R′″₄, independent of each other, each represents a hydrogen atom, an unsubstituted or at least mono-substituted, saturated or unsaturated linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted saturated, unsaturated or aromatic heterocyclic radical having from 5 to 14 atoms that can contain 1, 2 or 3 heteroatoms independently selected from O, N and S;
R′₅, R″₅, R′″₅, R′₆, R″₆ and R′″₆, independent of each other, each represents a hydrogen atom; an unsubstituted or at least mono-substituted, saturated or unsaturated, linear or branched C₁₋₁₈ aliphatic radical; an unsubstituted or at least mono-substituted C₃₋₁₂ cycloalkyl radical; an unsubstituted or at least mono-substituted mono- or polycyclic aryl radical; an unsubstituted or at least mono-substituted saturated, unsaturated or aromatic heterocyclic radical having from 5 to 10 atoms that can contain 1, 2 or 3 heteroatoms independently selected from O, N and S;
or
R′₅, R″₅, R′″₅, R′₆, R″₆ and R′″₆, form together with the bridging nitrogen atom an unsubstituted or at least mono-substituted, saturated, unsaturated or aromatic mono- or polycyclic ring system which has 5 to 10 atoms as ring members and which contains optionally 1, 2 or 3 heteroatoms independently selected from O, N and S;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

3. A combination according to claim 1 or 2, characterized in that the UV-absorbing active substance (A) is at least one heptaazaphenalene compound of general formula (IA) 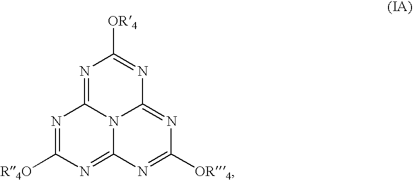
wherein
R′₄, R″₄ and R′″₄, independent of each other, each represents an unsubstituted or at least mono-substituted, C₃₋₁₂ cycloalkyl radical; an unsubstituted or at least mono-substituted mono- or polycyclic aryl radical; an unsubstituted or at least mono-substituted saturated, unsaturated or aromatic heterocyclic radical having from 5 to 14 atoms that can contain 1, 2 or 3 heteroatoms independently selected from O, N and S;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

4. A combination according to claim 1 or 2, characterized in that the UV-absorbing active substance (A) is at least one heptaazaphenalene compound of general formula (IB) 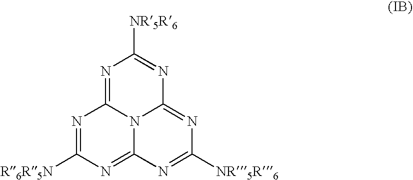
wherein
the radicals within each radical pair R′₅ R′₆, respectively, R″₅ R″₆, respectively, R′″₅R′″₆, different from each other, each represents a hydrogen atom; an unsubstituted or at least mono-substituted, linear or branched C₁₋₁₈ alkyl radical; an unsubstituted or at least mono-substituted C₃₋₁₂ cycloalkyl radical; an unsubstituted or at least mono-substituted mono- or polycyclic aryl radical; an unsubstituted or at least mono-substituted, saturated, unsaturated or aromatic heterocyclic radical having from 5 to 10 atoms that can contain 1, 2 or 3 heteroatoms independently selected from O, N and S;
or
the radical pair R′₅ R′₆, respectively, R″₅ R″₆, respectively, R′″₅R′″₆ form together with the bridging nitrogen atom an unsubstituted or at least mono-substituted, saturated, unsaturated or aromatic mono- or polycyclic ring system having from 4 to 10 atoms as ring members that contains optionally 1 or 2 heteroatoms selected from N, O and S;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

5. A combination according to claim 1 or 2, characterized in that the UV-absorbing active substance (A) is at least one heptaazaphenalene compound of general formula (IC) 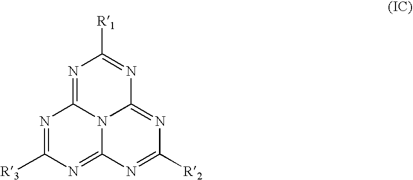
wherein
R′₁, R′₂ and R′₃, identical or different, each represents an unsubstituted or at least mono-substituted mono- or polycyclic aryl radical; an unsubstituted or at least mono-substituted saturated, unsaturated or aromatic heterocyclic radical having from 5 to 10 atoms and that can contain 1, 2 or 3 heteroatoms independently selected from O, N and S;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

6. A combination according to claim 5, characterized in that the UV-absorbing active substance (A) is at least one heptaazaphenalene compound of general formula (YA) 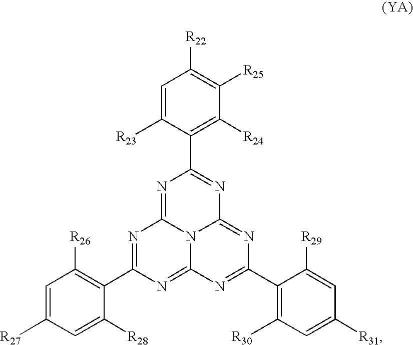
wherein
R₂₂, R₂₃, R₂₄, R₂₅, R₂₆, R₂₇, R₂₈, R₂₉, R₃₀ and R₃₁ have the following meaning:

| R₂₂ | R₂₃ | R₂₄ | R₂₅ | R₂₆ | R₂₇ | R₂₈ | R₂₉ | R₃₀ | R₃₁ |
|---|---|---|---|---|---|---|---|---|---|
| —O—CH₃ | H | H | H | —OH | 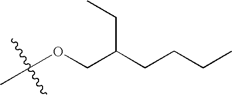 | H | —OH | H | 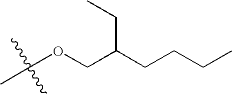 |
| 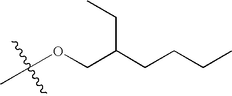 | —OH | H | H | —CH₃ | —CH₃ | H | —CH₃ | H | —CH₃ |
| 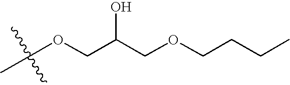 | —OH | H | H | —CH₂ | —CH₃ | H | —CH₃ | H | —CH₃ |
| 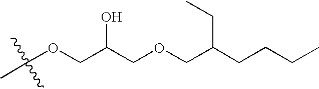 | —OH | H | H | —CH₃ | —CH₃ | H | —CH₃ | H | —CH₃ |
| 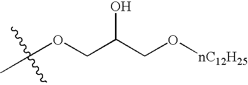 | —OH | H | H | —CH₃ | —CH₃ | H | —CH₃ | H | —CH₃ |
| 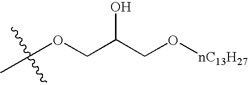 | —OH | H | H | —CH₃ | —CH₃ | H | —CH₃ | H | —CH₃ |
| 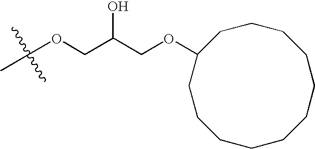 | —OH | H | H | —CH₃ | —CH₃ | H | —CH₃ | H | —CH₃ |
| 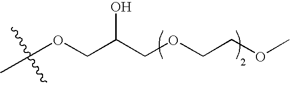 | —OH | H | H | —CH₃ | —CH₃ | H | —CH₃ | H | —CH₃ |
| 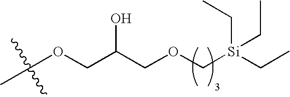 | —OH | H | H | —CH₃ | —CH₃ | H | —CH₃ | H | —CH₃ |
| 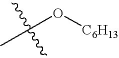 | H | H | H | H | H | H | H | H | H |
| —OAc | —OAc | H | H | H | H | H | H | H | H |
| 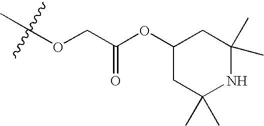 | H | H | H | —CH₃ | —CH₃ | H | —CH₃ | H | —CH₃ |
| 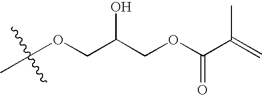 | H | H | H | —CH₃ | —CH₃ | H | —CH₃ | H | —CH₃ |
| —O—CH₃ | —OH | H | H | H | -Ph | H | H | H | -Ph |
| —OBu | —OH | H | H | H | -Ph | H | H | H | -Ph |
| 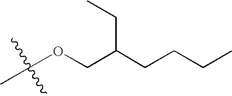 | —OH | H | H | H | -Ph | H | H | H | -Ph |
| 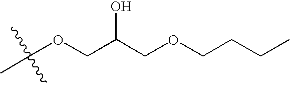 | —OH | H | H | H | -Ph | H | H | H | -Ph |
| 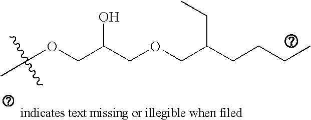 | —OH | H | H | H | -Ph | H | H | H | -Ph |
| 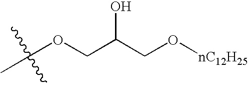 | —OH | H | H | H | -Ph | H | H | H | -Ph |
| 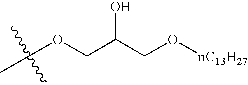 | —OH | H | H | H | -Ph | H | H | H | -Ph |
| 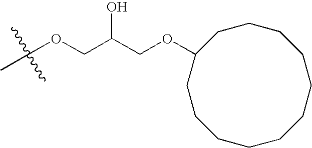 | —OH | H | H | H | -Ph | H | H | H | -Ph |
| 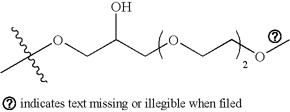 | —OH | H | H | H | -Ph | H | H | H | -Ph |
| 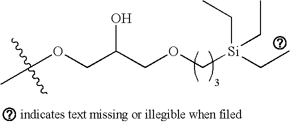 | —OH | H | H | H | -Ph | H | H | H | -Ph |
| 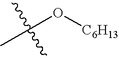 | H | H | H | H | -Ph | H | H | H | -Ph |
| 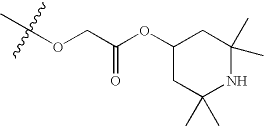 | H | H | H | H | -Ph | H | H | H | -Ph |
| 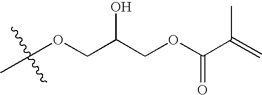 | H | H | H | H | -Ph | H | H | H | -Ph |
| H | H | H | H | H | H | H | H | H | H |
| H | —O—CH₃ | H | H | H | H | H | H | H | H |
| —O—CH₃ | H | H | H | H | H | H | H | H | H |
| —O—CH₃ | —OH | H | H | H | H | H | H | H | H |
| —O—CH₃ | OH | H | H | H | —CH₃ | H | H | H | —CH₃ |
| —O—CH₃ | —OH | H | H | —CH₃ | —CH₃ | H | —CH₃ | H | —CH₃ |
| —O—CH₃ | —OH | H | H | —O—CH₃ | —O—CH₃ | H | —O—CH₃ | H | —O—CH₃ |
| —OC₆H₁₃ | —OH | H | H | —CH₃ | —CH₃ | H | —CH₃ | H | —CH₃ |
| —OC₆H₁₃ | —O—CH₃ | H | H | H | H | H | H | H | H |
| —OC₆H₁₃ | —OH | H | H | H | —O—CH₃ | H | H | H | —O—CH₃ |
| —OC₆H₁₃ | —OH | H | H | —CH₃ | —CH₃ | —CH₃ | —CH₃ | —CH₃ | —CH₃ |
| —OC₆H₁₃ | —O—CH₃ | H | H | —CH₃ | —CH₃ | —CH₃ | —CH₃ | —CH₃ | —CH₃ |
| —OC₆H₁₃ | —OH | H | —CH₃ | H | H | H | H | H | H |
| —OC₆H₁₃ | —OH | —CH₃ | H | H | H | H | H | H | H |
| 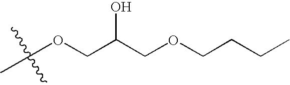 | —OH | H | H | —OH | 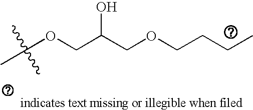 | H | H | H | H |
| 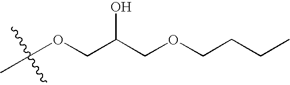 | —OH | H | H | —OH | 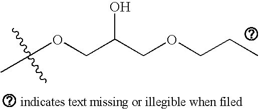 | H | —CH₃ | H | —CH₃ |
| 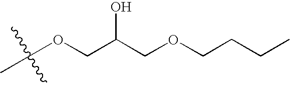 | —OH | H | H | —OH | 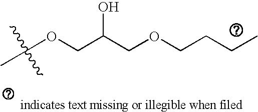 | H | —CH₃ | —CH₃ | —CH₃ |
| —O—CH₃ | —OH | H | H | —OH | 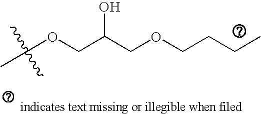 | H | —OH | H | 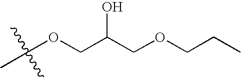 |
| 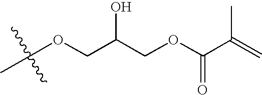 | —OH | H | H | —CH₃ | —CH₃ | H | —CH₃ | H | —CH₃ |
| —OC₁₂H₂₅ | —OH | H | H | —CH₃ | —CH₃ | H | —CH₃ | H | —CH₃ |
| —O—CH₃ | —O—CH₃ | H | H | H | —CH₃ | H | H | H | —CH₃ |
| —O—CH₃ | —O—CH₃ | H | H | —CH₃ | —CH₃ | H | —CH₃ | H | —CH₃ |
| —OC₁₂H₂₅ | —O—CH₃ | H | H | H | H | H | H | H | H |
| —OC₁₂H₂₅ | —O—CH₃ | H | H | H | —CH₃ | H | H | H | —CH₃ |
| —OC₁₂H₂₅ | —O—CH₃ | H | H | —CH₃ | —CH₃ | H | —CH₃ | H | —CH₃ |
| H | H | H | H | —OH | 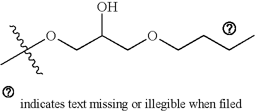 | H | —OH | H | 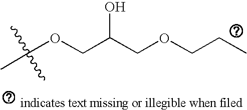 |
| —O—CH₃ | —O—CH₃ | H | H | H | H | H | H | H | H |
| —OC₁₂H₂₅ | —O—CH₃ | H | H | H | H | H | H | H | H |
| —O—CH₃ | —O—CH₃ | H | H | H | —CH₃ | H | H | H | —CH₃ |
| —OC₆H₁₃ | —O—CH₃ | H | H | H | —CH₃ | H | H | H | —CH₃ |
| —O—CH₃ | —O—CH₃ | H | H | —CH₃ | —CH₃ | H | H | H | —CH₃ |
| —OC₁₂H₂₅ | —O—CH₃ | H | H | —CH₃ | —CH₃ | H | —CH₃ | H | —CH₃ |
| H | —O—CH₃ | H | H | H | H | H | —CH₃ | H | H |
| —O—CH₃ | H | H | H | H | H | H | H | H | H |
| —O—CH₃ | H | H | H | H | —O—CH₃ | H | H | H | H |
| —O—CH₃ | H | H | H | H | —O—CH₃ | H | H | H | —O—CH₃ |
| H | —OH | H | H | H | H | H | H | H | H |
| H | —OH | H | H | —OH | H | H | H | H | H |
| H | H | H | H | H | H | H | H | H | H |
| 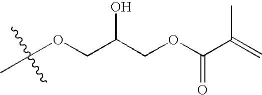 | —OH | H | H | —CH₃ | —CH₃ | H | —CH₃ | H | —CH₃e |
| —O—CH₃ | —OH | H | H | —O—CH₃ | —O—CH₃ | H | —O—CH₃ | H | —O—CH₃ |
| —O—CH₃ | —OH | H | H | —OH | —O—CH₃ | H | —O—CH₃ | H | —O—CH₃ |
| —O—CH₃ | —OH | H | H | H | H | H | H | H | H |
| 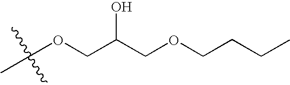 | —OH | H | H | —OH | 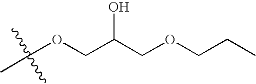 | H | H | H | H |

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

7. A combination according to claim 5, characterized in that the UV-absorbing active substance (A) is at least one heptaazaphenalene compound of general formula (YB) 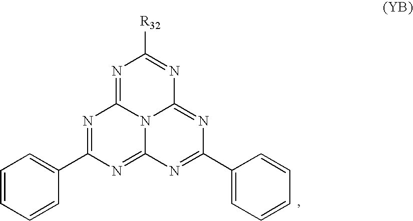
wherein
R₃₂ represents a radical selected from the group consisting of 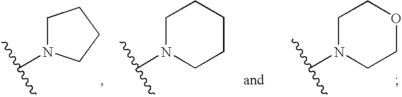
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

8. A combination according to claims 1 or 2, characterized in that the UV-absorbing active substance (A) is at least one heptaazaphenalene compound of general formula (YC) 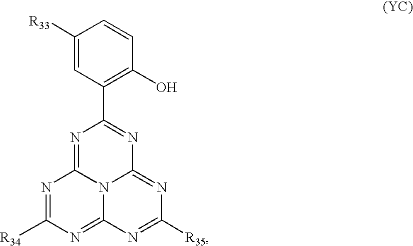
wherein R₃₃, R₃₄ and R₃₅ have the following meaning:

| R₃₃ | R₃₄ | R₃₅ |
|---|---|---|
| H | —O—CH₃ | —O—CH₃ |
| —CH₃ | —O—CH₃ | —O—CH₃ |
| —CH₃ | —O—CH₃ | —N(Me)₂ |
| —CH₃ | —N(Me)₂ | —N(Me)₂ |

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

9. A combination according to claims 1 or 2, characterized in that the UV-absorbing active substance (A) is at least one heptaazaphenalene compound of general formula (YD) 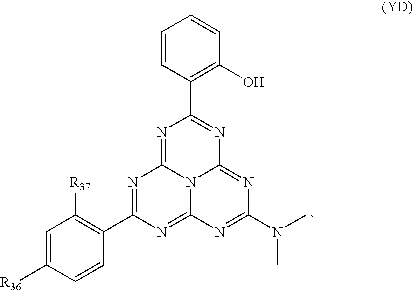
wherein
R₃₆ and R₃₇ have the following meaning:

| R₃₆ | R₃₇ |
|---|---|
| H | H |
| H | —OH |
| —CH₃ | H |

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

10. A combination according to claims 1 or 2, characterized in that the UV-absorbing active substance (A) is a heptaazaphenalene compound of formula (YE) 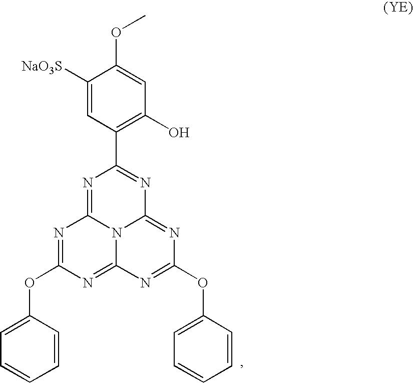
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

11. A combination according to claim 5, characterized in that the UV-absorbing active substance (A) is at least one heptaazaphenalene compound of general formula (YF) 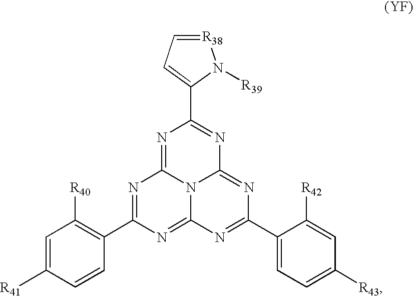
wherein
R₃₈, R₃₉, R₄₀, R₄₁, R₄₂ and R₄₃ have the following meaning:

| | | | | | |
|---|---|---|---|---|---|
| R₃₈ | R₃₉ | R₄₀ | R₄₁ | R₄₂ | R₄₃ |
| | | | | | |
|---|---|---|---|---|---|
| N | —CH₃ | —OH | —OH | —OH | —OH |
| N | —CH₃ | —OH | 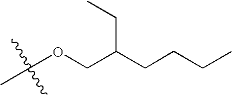 | —OH | 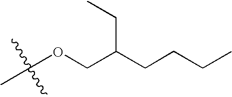 |
| N | —CH₃ | 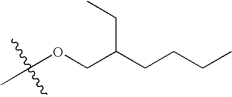 | 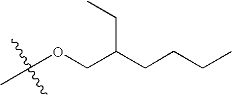 | —OH | 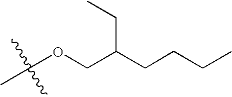 |
| N | -Ph | —OH | —OH | —OH | —OH |
| N | -Ph | —OH | 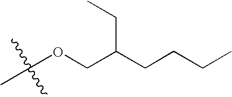 | —OH | 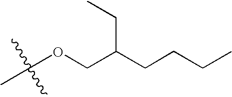 |
| —CH | —CH₂-Ph | —OH | —OH | —OH | —OH |
| —CH | —CH₂-Ph | —OH | 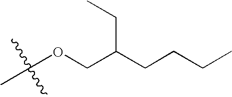 | —OH | 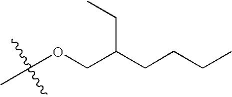 |

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

12. A combination according to claims 1 or 2, characterized in that the UV-absorbing active substance (A) is at least one heptaazaphenalene compound of general formula (YG) 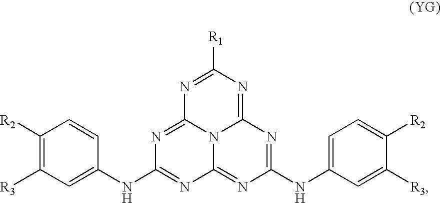
wherein
R₄₄, R₄₅ and R₄₆ have the following meaning:

| R₄₄ | R₄₅ | R₄₆ |
|---|---|---|
| 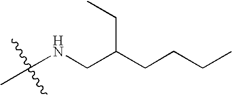 | 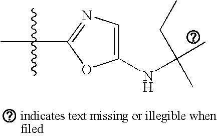 | H |
| 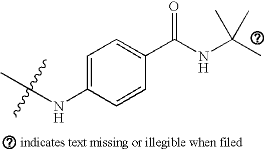 | 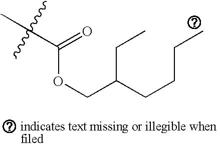 | H |
| 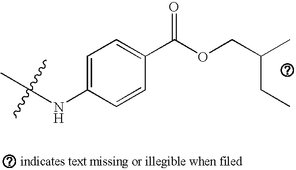 | 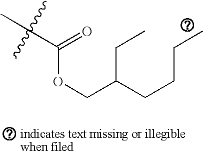 | H |
| 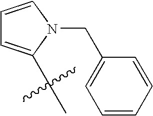 | -Ph | H |
| 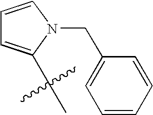 | 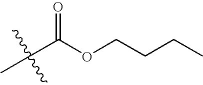 | H |
| 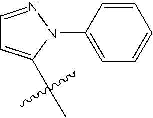 | 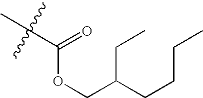 | H |
| 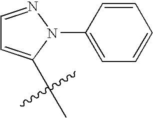 | 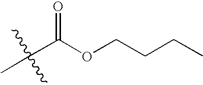 | H |
| 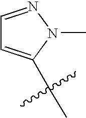 | 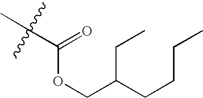 | H |
| 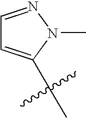 | 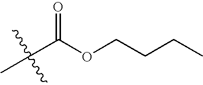 | H |
| 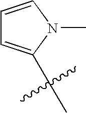 | 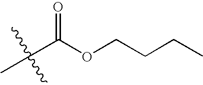 | H |
| 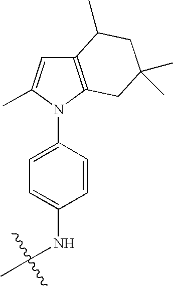 | 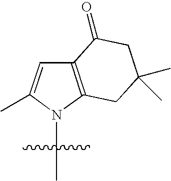 | H |
| 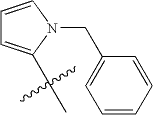 | —C—O-Ph | H |

and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt thereof, or at least one corresponding solvate thereof;
which active substance (A) has its absorption maximum λ_{max(A)} in the range of either UV-A or UV-B radiation
and the UV-absorbing active substance (B)
is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

13. A combination according to at least one of the preceding claims 1 to 12, characterized in that the UV-absorbing active substance (A) is at least one heptaazaphenalene compound selected from the group comprising
2,5,8-tris-(4-(butoxycarbonyl)phenylamino)-1,3,4,6,7,9,9b-heptaazaphenalene,
2,5,8-tris-(4-(2-ethylhexyloxycarbonyl)phenylamino)-1,3,4,6,7,9,9b-heptaazaphenalene,
2,5,8-tris-(4-(imidazo[1,2-a]pyridin-2-yl)-phenylamino)-1,3,4,6,7,9,9b-heptaazaphenalene,
2,5,8-tris-(4-(4,5,6,7-tetrahydro-2,6,6-trimethyl-4-oxoindol-1-yl)phenylamine)-1,3,4,6,7,9,9b-heptaazaphenalene,
2,5,8-tris-(biphenyl-4-ylamino)-1,3,4,6,7,9,9b-heptaazaphenalene,
2,5,8-tris-(4-(1H-benzo[d]imidazol-2-yl)phenylamino)-1,3,4,6,7,9,9b-heptaazaphenalene,
2,5-bis-(biphenyl-4-ylamino)-8-(4-(butoxycarbonyl)phenylamino)-1,3,4,6,7,9,9b-heptaazaphenalene,
2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene,
2,5,8-tris-(2,4-dihydroxyphenyl)-1,3,4,6,7,9,9b-heptaazaphenalene,
2,5-dichloro-8-(1-methyl-1H-pyrrole-2-yl)-1,3,4,6,7,9,9b-heptaazaphenalene,
2,5-bis-(4-(butoxycarbonyl)phenylamino)-8-(2-(1-methyl-1H-pyrrole-2-yl)-1,3,4,6,7,9,9b-heptaazaphenalene,
2-(4-(carboxy)phenylamino)-5,8-bis-(4-methylphenyl)-1,3,4,6,7,9,9b-heptaazaphenalene,
2,5,8-tris-(4-(4,5,6,7-tetrahydro-2,6,6-trimethyl-4-oxoindol-1-yl)phenylamine)-1,3,4,6,7,9,9b-heptaazaphenalene,
2,5-bis-(4-(butoxycarbonyl)phenylamino)-8-(1-methyl-1H-pyrrole-2-yl)-1,3,4,6,7,9,9b-heptaazaphenalene,
2,5-bis(2,4-dihydroxyphenyl)-8-(1-methyl-1H-pyrazol-5-yl)-1,3,4,6,7,9,9b-heptaazaphenalene,
2,5-bis{[4-(2-ethylhexyloxy)-2-hydroxy]phenyl}-6-(1-methyl-1H-pyrazol-5-yl)-1,3,4,6,7,9,9b-heptaazaphenalene,
2-[2,4-bis(2-ethylhexyloxy)phenyl]-5-[4-(2-ethylhexyl oxy)-2-hydroxyphenyl]-8-(1-methyl-1H-pyrazol-5-yl)-1,3,4,6,7,9,9b-heptaazaphenalene,
2,5-bis(2,4-dihydroxyphenyl)-8-(1-phenyl-1H-pyrazol-5-yl)-1,3,4,6,7,9,9b-heptaazaphenalene,
2,5-bis{[4-(2-ethylhexyloxy)-2-hydroxy]phenyl}-8-(1-phenyl-1H-pyrazol-5-yl)-1,3,4,6,7,9,9b-heptaazaphenalene,
2,5-bis[4-(butoxycarbonyl)phenylamino]-8-(1-methyl-1H-pyrazol-5-yl)-1,3,4,6,7,9,9b-heptaazaphenalene,
2,5-bis{4-[(2-(ethylhexyloxy)carbonyl]phenylamino}-8-(1-methyl-1H-pyrazol-5-yl)-1,3,4,6,7,9,9b-heptaazaphenalene,
2,5-bis[4-(butoxycarbonyl)phenylamino]-8-(1-phenyl-1H-pyrazol-5-yl)-1,3,4,6,7,9,9b-heptaazaphenalene,
2,5-bis{4-[(2-ethylhexyloxy)carbonyl]phenylamino}-8-(1-phenyl-1H-pyrazol-5-yl)-1,3,4,6,7,9,9b-heptaazaphenalene,
2-(1-benzyl-1H-pyrrole-2-yl)-5,8-bis(2,4-dihydroxyphenyl)-1,3,4,6,7,9,9b-heptaazaphenalene,
2-(1-benzyl-1H-pyrrole-2-yl)-5,8-bis[4-(2-ethylhexyloxy)-2-hydroxyphenyl]-1,3,4,6,7,9,9b-heptaazaphenalene,
2-(1-benzyl-1H-pyrrole-2-yl)-5,8-bis[4-(butoxycarbonyl)phenylamino]-1,3,4,6,7,9,9b-heptaazaphenalene,
2-(1-benzyl-1H-pyrrole-2-yl)-5,8-bis(biphenyl-4-ylamino)-1,3,4,6,7,9,9b-heptaazaphenalene,
2-(1-benzyl-1H-pyrrole-2-yl)-5,8-bis(4-benzoylphenylamino)-1,3,4,6,7,9,9b-heptaazaphenalene,
2-(1-benzyl-1H-pyrrole-2-yl)-5,8-bis(9-oxo-9H-fluoren-3-ylamino)-1,3,4,6,7,9,9b-heptaazaphenalene,
2-(4-(tert-butylcarbamoyl)phenylamino)-5,8-bis-(4-(2-ethylhexyloxycarbonyl)phenylamino)-1,3,4,6,7,9,9b-heptaazaphenalene,
2,5-bis-[(4-(2-ethylhexyloxy)-2-hydroxy)-phenyl]-8-(4-methoxyphenyl)-1,3,4,6,7,9,9b-heptaazaphenalene,
2-(2-ethylhexylamino)-5,8-bis-(4-(5-(1,1-dimethylpropyl)benzo[d]oxazol-2-yl)phenylamino)-1,3,4,6,7,9,9b-heptaazaphenalene,
2,5,8-tris-(4-(1H-pyrazol-1-yl)phenylamino)-1,3, 4, 6, 7, 9,9b-heptaazaphenalene
2,5,8-tris-(4-benzoylphenylamino)-1,3,4,6,7,9,9b-heptaazaphenalene,
2,5,8-tris-(4-butoxy-2-hydroxyphenyl)-1,3,4,6,7,9,9b-heptaazaphenalene,
2,5,8-tris-(naphthalen-2-ylamino)-1,3,4,6,7,9,9b-heptaazaphenalene,
2,5-bis-(4-(butoxycarbonyl)phenylamino)-8-(2-(1-methyl-1H-indol-3-yl)-1,3,4,6,7,9,9b-heptaazaphenalene,
2,5,8-tris-(biphenyl-4-yloxy)-1,3,4,6,7,9,9b-heptaazaphenalene,
2,5,8-tris-(3-methoxyphenylamino)-1,3,4,6,7,9,9b-heptaazaphenalene,
2,5,8-tris-(4-methoxyphenylamino)-1,3,4,6,7,9,9b-heptaazaphenalene,
2,5,8-tris-(4-((E)-3-ethoxy-3-oxoprop-1-enyl)phenylamino)-1,3,4,6,7,9,9b-heptaazaphenalene,
2,5,8-tris-(methoxycarbonyl-4′-biphenyl-4-ylamino)-1,3,4,6,7,9,9b-heptaazaphenalene,
2,5,8-tris-(4-(methoxycarbonyl)phenylamino)-1,3,4,6,7,9,9b-heptaazaphenalene,
2,5,8-tris-(4-(1H-benzo[d]imidazol-2-yl)-3-hydroxyphenylamino)-1,3,4,6,7,9,9b-heptaazaphenalene,
2,5,8-tris-(4-(phenylamino)phenylamino)-1,3,4,6,7,9,9b-heptaazaphenalene,
2,5,8-tris-((4-(E)-styrylphenyl)amino)-1,3,4,6,7,9,9b-heptaazaphenalene,
2,5,8-tris-(2-ethylhexylamino)-1,3,4,6,7,9,9b-heptaazaphenalene,
2,5,8-tris-(4-(L-menthylcarbonyl)phenylamino)-1,3,4,6,7,9,9b-heptaazaphenalene,
2,5,8-tris-(4-((3,3,5-trimethylcyclohexyloxy)carbonyl)phenylamino)-1,3,4,6,7,9,9b-heptaazaphenalene,
2,5,8-tris-(4-((E)-3-(2-ethylhexyloxy)-3-oxoprop-1-enyl)phenylamino)-1,3,4,6,7,9,9b-heptaazaphenalene,
2-(3-((E)-3-(2-ethylhexyloxy)-3-oxoprop-1-enyl)phenylamino)-5,8-bis-(4-((E)-3-(2-ethylhexyloxy)-3-oxoprop-1-enyl)phenylamino)-1,3,4,6,7,9,9b-heptaazaphenalene,
2,5,8-tris-(3-nitrophenylamino)-1,3,4,6,7,9,9b-heptaazaphenalene,
2,5,8-tris-(4-(2-butyloctyloxycarbonyl)phenylamino)-1,3,4,6,7,9,9b-heptaazaphenalene,
2,5,8-tris-(4-(2-hexyldecyloxycarbonyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(hexyldecyloxycarbonyl)phenylamino)-1,3,4,6,7,9,9b-heptaazaphenalene,
2,5,8-tris-(4-(octyldecyloxycarbonyl)phenylamino)-1,3,4,6,7,9,9b-heptaazaphenalene,
2,5,8-tris-((3-(E)-styrylphenyl)amino)-1,3,4,6,7,9,9b-heptaazaphenalene,
2,5,8-tris-(4-((3,5,5-trimethylhexyloxy)carbonylphenylamino)-1,3,4,6,7,9,9b-heptaazaphenalene,
2,5-bis-(3-(methoxy)phenylamino)-8-(2-(1-methyl-1H-pyrrole-2-yl)-1,3,4,6,7,9,9b-heptaazaphenalene,
2,5,8-tris-(4-((2-ethylhexyl)carbamoyl)phenylamino)-1,3,4,6,7,9,9b-heptaazaphenalene,
2,5,8-tris-(4-(dodecyloxycarbonyl)phenylamino)-1,3,4,6,7,9,9b-heptaazaphenalene,
2,5,8-tris-(4-(1,3,3-trimethylbicyclo[2.2.1]heptan-2-yloxy)phenylamino)-1,3,4,6,7,9,9b-heptaazaphenalene,
2,5,8-tris-(1H-indol-5-ylamino)-1,3,4,6,7,9,9b-heptaazaphenalene,
2,5,8-tris-(4-((3,7-dimethyloctyloxy)carbonyl)phenylamino)-1,3,4,6,7,9,9b-heptaazaphenalene and
2,5,8-tris-(2-amino-4,5-dimethylphenylamino)-1,3,4,6,7,9,9b-heptaazaphenalene;
and/or at least one of the stereoisomers, preferably enantiomers or diastereomers, a racemate or a mixture of at least two of its stereoisomers, preferably enantiomers and/or diastereomers in any mixing ratio, or at least one physiologically acceptable salt, or at least one corresponding solvate of the aforementioned compounds;
which active substance (A) has its absorption maximum λ_{max(A)} in the range of either UV-A or UV-B radiation
and
the UV-absorbing active substance (B) is at least one compound as defined in claim 1, wherein the ratio of the molar extinction coefficient at λₘₐₓ [ε_{λmax(A)}] of the UV-absorbing active substance (A) to the molar extinction coefficient at λₘₐₓ [ε_{λmax(B)}] of the UV-absorbing active substance (B) is from 1:10 to 50:1.

14. A combination according to anyone of the preceding claims, characterized in that the UV-absorbing active substance (B) has its absorption maximum λ_{max(B)} in the range of 290 to 400 nm.

15. A combination according to at least one of the preceding claims 1 to 13, characterized in that λ_{max(B)} of active substance (B) does not lie in the same UV radiation range wherein the λ_{max(A)} of active substance (A) lies.

16. A combination according to at least one of the preceding claims, characterized in that the UV-absorbing active substance (B) is at least one organic and/or at least one inorganic compound.

17. A combination according to claim 16, characterized in that the UV-absorbing active substance (B) is at least one compound selected from the group comprising substituted acrylic acid derivatives, allantoin derivatives, aminobenzoic acid derivatives, aminobenzamides derivatives, anthranilic acid derivatives, benzalmalonic acid derivatives, benzimidazole derivatives, benzoic acid derivatives, benzophenone derivatives, benzothiazole derivatives, benzotriazole derivatives, benzoxazole derivatives, camphor derivatives, cinnamic acid derivatives, coumarinic acid derivatives, curcumin derivatives, dianisoylmethane derivatives, dibenzalazine derivatives, dibenzoylmethane derivatives, dioxane derivatives, fungi extract, ferulic acid derivatives, furane derivatives, glutamic acid derivatives, imidazoline derivatives, malonic acid derivatives, metal oxides, metal dioxides, nicotinic acid derivatives, nitrobenzamides derivatives, nitrobenzoic acid derivatives, nucleic acid derivatives, p-aminobenzoic acid (PABA) derivatives, plant extracts, propenoate derivatives, pyrimidine derivatives, salicylic acid derivatives, tetrazole derivatives, triazine derivatives, tributamine derivatives, urocanic acid derivatives and vitamin B₆ derivatives;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned substances;

18. A combination according to claim 16, characterized in that the UV-absorbing active substance (B) is at least one compound of general formula (M) 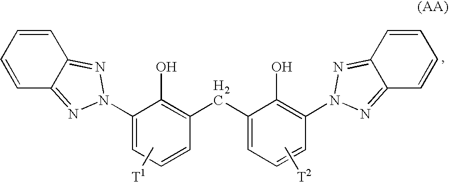
wherein
T¹ and T², independent of each other, each represents a C₁₋₈ alkyl radical which can be unsubstituted or at least mono-substituted with radicals independently selected from the group consisting of C₁₋₄ alkyl, C₅₋₁₂ cycloalkyl and aryl;
and/or
at least one compound of general formula (AB) 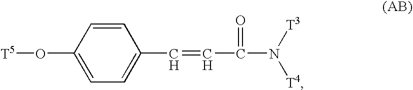
wherein
T³ represents a —(CONH)ᵣ-phenyl moiety, in which s is 0 or 1 and the phenyl group is unsubstituted or at least mono-substituted with radicals independently selected from the group consisting of —OH, C₁₋₁₈ alkyl and —O—C₁₋₈ alkyl, or a —C(═O)—OT^{3′} moiety, in which T^{3′} is a C₁₋₁₈ alkyl radical;
T⁴ represents a hydrogen atom or a C₁₋₄ alkyl radical; and
T⁵ represents H or a linear or branched —C₁₋₁₈ alkyl radical;
and/or
at least one compound of general formula (AC) 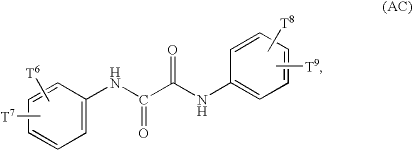
wherein
T⁶, T⁷, T⁸ and T⁹, independent of each other, each represents a C₁₋₈ alkyl radical or an —O—C₁₋₈ alkyl radical;
and/or
at least one compound of general formula (AD) 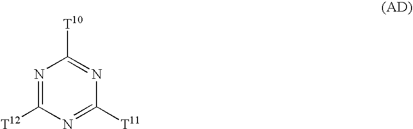
wherein
T¹⁰, T¹¹ and T¹², independent of each other, each represents a radical selected from the group consisting of —NR^{T10}R^{T11}, phenyl, —O-phenyl or pyrrolyl, which is unsubstituted or at least mono-substituted with 1, 2 or 3 substituent(s) independently selected from the group consisting of —OH, —C₁₋₁₈ alkyl, —O—C₁₋₁₈ alkyl, —(C═O)—C₁₋₁₈ alkyl, C₅₋₈ cycloalkyl, a methylidenecamphor group or a —(CH═CH)ₙ(CO)—OT^{10′}, with T^{10′} being either C₁₋₁₈ alkyl or cinnamyl, and n is 0 or 1;
wherein R^{T10} and R^{T11}, independent of each other, each represents a hydrogen atom; a linear or branched, unsubstituted or at least unsubstituted C₁₋₁₈ alkyl radical; an unsubstituted or at least unsubstituted aryl radical or an unsubstituted or at least unsubstituted —(C═O)—C₁₋₁₈ alkyl radical;
and/or
at least one compound of general formula (AE) 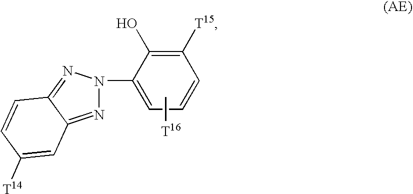
wherein
T¹⁴ represents a hydrogen atom or a C₁₋₁₈ alkyl radical;
T¹⁵ represents a C₁₋₈ alkyl radical which can be unsubstituted, an at least mono-substituted with a phenyl radical or a radical of general formula (VI′) 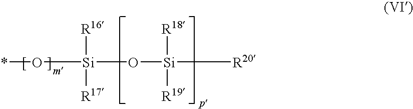
wherein
m′=0 or 1;
p′=0, 1, 2, 3 or 4;
R^{16′}, R^{17′}, R^{18′}, R^{19′} and R^{20′}, independent of each other, each represents an unsubstituted or at least mono-substituted C₁₋₆ alkyl radical; an unsubstituted or at least mono-substituted —O—C₁₋₆ radical; an unsubstituted or at least mono-substituted aryl radical; or an —O—Si(R^{21′})₃ radical with R^{21′} representing an unsubstituted or at least mono-substituted C₁₋₆ alkyl radical; an —O—C₁₋₆ radical; an unsubstituted or at least mono-substituted aryl radical;
T¹⁶ represents a C₁₋₈ alkyl radical, which can be unsubstituted or an at least mono-substituted with a phenyl radical;
and/or
at least one compound of general formulae (AF), (AG) and (AH) 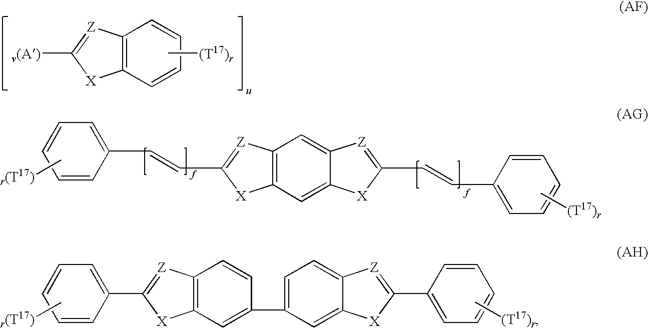
wherein
each of the symbols X, independently from each other represents an oxygen or sulfur atom or a group NT^{17′}, each of the symbols Z, independently from each other represents a nitrogen atom or a CH group, each of the symbols T¹⁷, independently from each other represents an —OH group, a halogen atom, a linear or branched C₁₋₈ alkyl radical optionally containing a silicon atom, or a linear or branched —O—C₁₋₈ alkyl radical, each of the numbers r is independently from each other 0, 1 or 2, u represents an integer ranging from 1 to 4 inclusive, v is equal to 0 or 1, each of the numbers r is independently from each other equal to 0 or 1, each of the symbols T^{17′} independently represents a hydrogen atom or a linear or branched C₁₋₈ alkyl radical or benzyl group optionally containing a silicon atom;
A′ represents a radical of valency u selected from the following group of formulae: 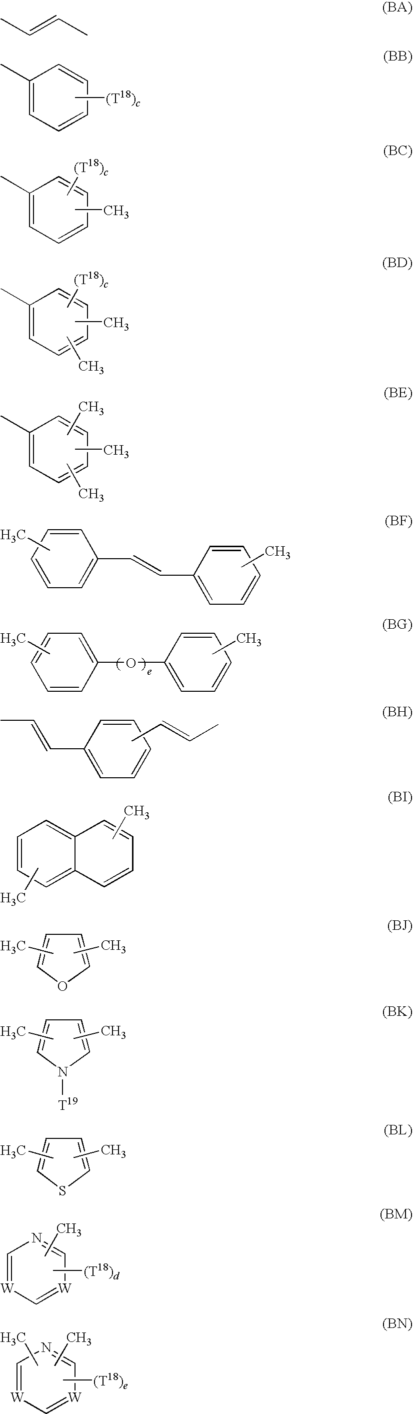 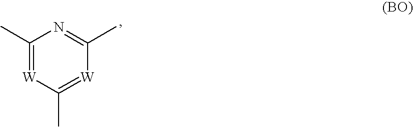
wherein
each of the symbols T¹⁸, independently from each other represents a halogen atom or a linear or branched C₁₋₄ alkyl or —O—C₁₋₄ alkyl radical, or —OH; T¹⁹ represents a hydrogen atom or a linear or branched C₁₋₄ alkyl radical; W represents a —CH— group or a nitrogen atom; c=0, 1, 2, 3 or 4; d=0, 1 or 2 and e=0 or 1;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds of general formulae (AA) to (AH).

19. A combination according to claim 16, characterized in that the UV-absorbing active substance (B) is at least one compound of the general formula (X) 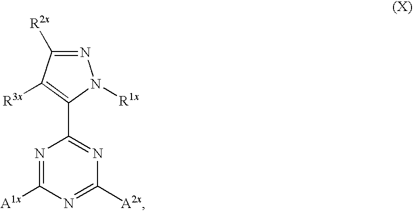
wherein
R^{1x} represents a hydrogen atom; an optionally substituted cycloalkyl radical from 3 to 7 carbon atoms; an aryl radical which is unsubstituted or at least mono-substituted with a group selected from aryl, halogen, an —O—C₁₋₆ radical and a C₁₋₆ alkyl radical; a-phenyl-C₁₋₆ alkyl radical; a linear or branched C₁₋₈ alkyl radical which is unsubstituted or at least mono-substituted with a moiety selected from —SO₃M^{x}, —N(R^{4x})₃⁺ and a radical of general formula (XX) 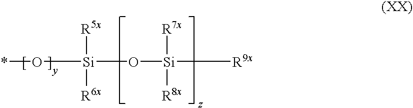
wherein
y=0 or 1;
z=0, 1, 2, 3 or 4;
R^{5x}, R^{6x}, R^{7x}, R^{8x} and R^{9x}, independent of each other, each represents an unsubstituted or at least mono-substituted C₁₋₆ alkyl radical; an —O—C₁₋₆ radical; an unsubstituted or at least mono-substituted aryl radical and an —OSi(R^{10x})₃ radical;
R^{10x} represents a C₁₋₆ alkyl radical; an —O—C₁₋₆ radical; or an unsubstituted or at least mono-substituted aryl radical;
M^{x} represents a hydrogen atom, Na⁺ or K⁺;
R^{4x} represents an unsubstituted or at least mono-substituted C₁₋₆ alkyl radical;
R^{2x} and R^{3x}, independent of each other, each represents a hydrogen atom; an unsubstituted or at least monosubstituted C₁₋₄ alkyl radical; or an unsubstituted or at least mono-substituted aryl radical;
A^{1x} represents a radical of general formula (X1) or (X2) 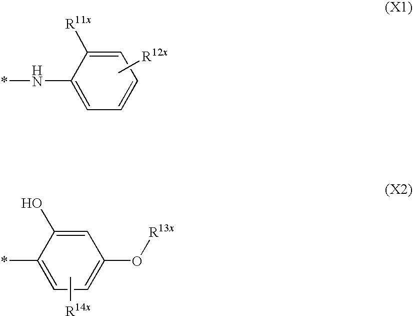
A^{2x} represents a radical of general formula (X1), (X3) or (X4) 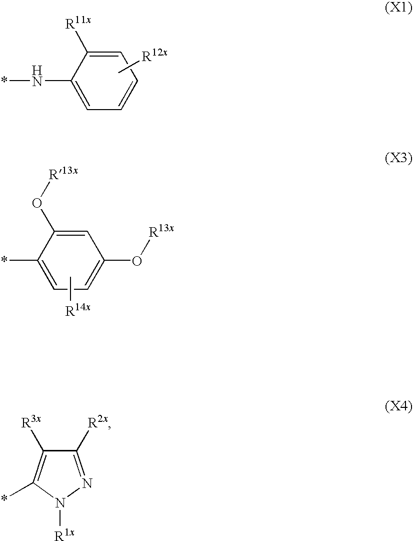
wherein
R^{11x} represents a hydrogen atom; a linear or branched, saturated or unsaturated, unsubstituted or at least monosubstituted C₁₋₆ aliphatic radical; or a hydroxy (—OH) residue;
R^{12x} represents a hydrogen atom; a —C(═O)—OR^{15x} radical; a —C(═O)—NR^{16x}R^{17x} radical; an unsubstituted or at least monosubstituted —O—C₁₋₁₈ radical; an unsubstituted or at least monosubstituted —O-phenyl radical; a C₅₋₇ cycloalkyl radical; a phenyl or a naphtyl radical; a phenyl or a naphtyl radical which are substituted by 1 or 2 substituents which are independently from one another selected from the group consisting of phenyl, Cl, —O—C₁₋₆ alkyl and C₁₋₆ alkyl; a linear or branched, saturated or unsaturated C₁₋₁₈ aliphatic radical which is unsubstituted or at least mono-substituted with —OH, -phenyl or un moiety of general formula (XX) as defined above;
R^{15x}, R^{16x} and R^{17x}, independent of each other, each represents a hydrogen atom; a linear or branched, unsubstituted or at least mono-substituted C₁₋₁₈ alkyl radical;
R^{14x} represents hydrogen atom; or —SO₃M^{x}, with M^{x} being H, Na⁺ or K⁺;
R^{13x} and R′^{13x}, independent of each other, each represents a hydrogen atom; an unsubstituted or at least monosubstituted C(═O)—C₁₋₁₈ radical; a linear or branched C₁₋₁₈ alkyl radical, which is unsubstituted or at least mono-substituted with at least one hydroxy (—OH) residue, a SO₃M^{x} moiety, a —N(R^{4x})₃⁺ moiety, wherein M^{x} and R^{4x} have the meaning as defined above, or a radical of general formula (XX) as defined above;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds of general formula (X);
and/or
at least one compound of general formula (Is) 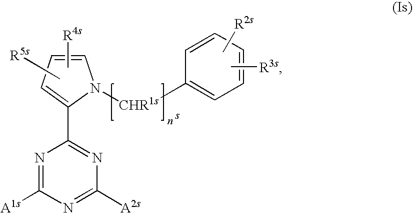
wherein
n^{s}=0, 1, 2, 3 or 4;
R^{1s} represents a hydrogen atom; a linear or branched, unsubstituted or at least monosubstituted C₁₋₃ alkyl radical; or a R^{2s}′R^{3s}′-substituted phenyl-radical;
R^{2s}, R^{2s′}, R^{3s} and R^{3s′}, independent of each other, each represents a hydrogen atom; a halogen atom; a hydroxy (—OH) residue; an unsubstituted or at least monosubstituted C₁₋₃ alkyl radical; an unsubstituted or at least monosubstituted —O—C₁₋₃ radical; or an unsubstituted or at least monosubstituted aryl radical;
or
R^{2s} and R^{3s} form together with the phenyl ring to which they are attached, an unsubstituted or at least mono-substituted naphthalene ring;
R^{4s} and R^{5s}, independent of each other, each represents a hydrogen atom; an unsubstituted or at least monosubstituted C₁₋₄ alkyl radical; or an unsubstituted or at least mono-substituted aryl radical;
A^{1s} represents a radical of general formula (IIs), (IIIs) or (IVs) 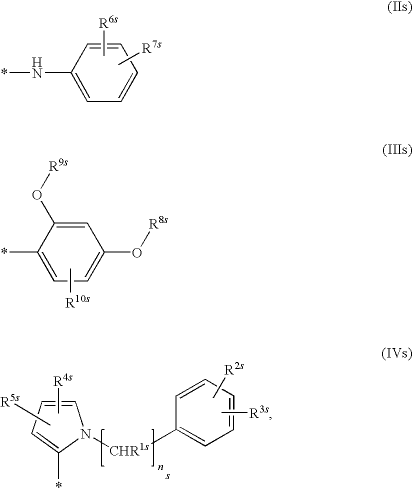
A^{2s} represents a radical of general formula (IIs) or (Vs) 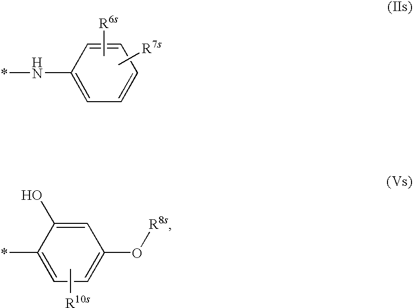
wherein
R^{6s} represents a hydrogen atom; a linear or branched, unsubstituted or at least monosubstituted C₁₋₆ alkyl radical; or a hydroxy (—OH) residue;
R^{7s} represents a hydrogen atom; an unsubstituted or at least monosubstituted, saturated or unsaturated, C₃₋₆ cycloalkyl radical; an unsubstituted or at least monosubstituted, saturated or unsaturated C₅₋₁₀ cycloalkyl radical having from 5 to 10 atoms that can contain 1, 2 or 3 heteroatom(s) as ring member(s) independently selected from the group consisting of O, N and S; an unsubstituted or at least monosubstituted aryl radical; an unsubstituted or at least monosubstituted heteroaryl radical having from 5 to 10 atoms that can contain 1, 2 or 3 heteroatom(s) as ring member(s) independently selected from the group consisting of O, N and S; a —C(═O)—OR^{11s} radical; a —C(═O)—NR^{12s}R^{13s} radical; an unsubstituted or at least monosubstituted —O—C₁₋₁₈ radical; an unsubstituted or at least monosubstituted —O-phenyl or —O-naphtyl radical; an unsubstituted or at least monosubstituted —COR^{14s} radical; a linear or branched, saturated or unsaturated, C₁₋₁₈ aliphatic radical, which is unsubstituted or at least mono-substituted with at least one hydroxy (—OH) residue, a SO₃M^{s} moiety, a —N(R^{15s})₃⁺ moiety or a radical of general formula (VIs) 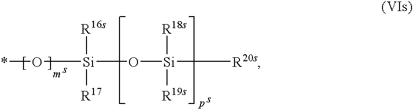
wherein
m^{s}=0 or 1;
p^{s}=0, 1, 2, 3 or 4;
R^{16s}, R^{17s}, R^{18s}, R^{19s} and R^{20s}, independent of each other, each represents an unsubstituted or at least mono-substituted C₁₋₆ alkyl radical; an unsubstituted or at least mono-substituted —O—C₁₋₆ radical; an unsubstituted or at least mono-substituted aryl radical; or an —O—Si(R^{21s})₃ radical;
R^{21s} represents an unsubstituted or at least mono-substituted C₁₋₆ alkyl radical; an —O—C₁₋₆ radical; an unsubstituted or at least mono-substituted aryl radical;
R^{11s}, R^{12s} and R^{13s}, independent of each other, each represents a hydrogen atom; a linear or branched, unsubstituted or at least monosubstituted C₁₋₁₈ alkyl radical; an unsubstituted or at least mono-substituted C₃₋₆ cycloalkyl radical;
or
R^{12s} and R^{13s} form together with the bridging nitrogen atom an unsubstituted or at least mono-substituted, saturated 5 to 7-membered cycloaliphatic radical, which contains 1, 2 or 3 heteroatom(s) as ring member(s) independently selected from the group consisting of O, N and S;
R^{14s} represents an unsubstituted or at least mono-substituted C₁₋₁₈ alkyl radical; or an unsubstituted or at least mono-substituted aryl radical;
R^{15s} represents an unsubstituted or at least mono-substituted C₁₋₁₈ alkyl radical;
M^{s} represents H, Na⁺ or K⁺;
R^{6s} and R^{7s} form together with the phenyl ring an unsubstituted or at least mono-substituted 9 to 15-membered polycyclic ring system;
or
R^{6s} and R^{14s} form together with the phenyl ring an unsubstituted or at least mono-substituted 9 to 15-membered polycyclic ring system;
R^{8s} and R^{9s}, independent of each other, each represents a hydrogen atom; an unsubstituted or at least monosubstituted —C(═O)—C₁₋₁₈ radical; a linear or branched, saturated or unsaturated C₁₋₁₈ aliphatic radical, which is unsubstituted or at least mono-substituted with at least one hydroxy (—OH) residue, a —SO₃M^{s} moiety, a —N(R^{15s})₃⁺ moiety or a radical of general formula (VIs) as defined above;
R^{10s} represents a hydrogen atom; or a —SO₃M^{s} moiety with M^{s} representing H, Na⁺ or K⁺;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds of general formula (Is).

20. A combination according to claims 16 to 18, characterized in that the UV-absorbing active substance (B) is at least one compound selected from the group comprising 2-ethylhexyl 2-cyano-3,3′-diphenylacrylate, ethyl 2-cyano-3,3′-diphenylacrylate, polymer of N-{(2 and 4)[(2-oxoborn-3-ylidene)methyl]benzyl}acrylamide, methyl 4-aminobenzoate, butyl 4-aminobenzoate, 5-methyl-2-(1-methylethyl)-cyclohexyl 2-aminobenzoate, glyceryl aminobenzoate, 2-ethylhexyl-p-dimethylethyl-aminobenzoate, 4-aminobenzamide, homomethyl-N-acetyl-anthranilic acid, menthyl antrhanilate, 2-(1H-benzimidazol-2-yl)-4-methoxyphenol, 2,2′-bis(benzimidazole), monosodium salt of 2-2′-bis-(1,4-phenylene)1H-benzimidazole-4,6-disulphonic acid, 2-phenylbenzimidazole-5-sulphonic acid, 5,5′,6,6′-tetramethyl-2,2′-bis(benzimidazole), 5,5′-dimethyl-2,2′-bis(benzimidazole), 6-methoxy-2,2′-bis(benzimidazole), 1,4-phenylenebis(2-benzimidazolyl), 1,2-phenylenebis)benzimidazolyl), 1,4-phenylenebis(N-2-ethylhexyl-2-benzimidazolyl), 1,4-phenylenebis(N-tri-methylsilylmethyl-2-benzimidazolyl), 2-(1H-benzimidazol-2-yl)benzothiazole, 2-(1H-benzimidazol-2-yl)benzoxazole, N,N′-dimethyl-2,2′-bis(benzimidazole), phenylbenzimidazole-5-sulfonic acid (Ensulizole) and its salts, 2-2′-bis-(1,4-phenylene)1H-benzimidazole-4,6-disulphonic acid, 3-methylbutyl 4-(dimethylamino)benzoate, 3,3,5-trimethylcyclohexyl 2-hydroxybenzoate (Homosalate), ethyl 4-aminobenzoate, 4,4-((6-(((1,1-dimethylethyl)amino)carbonyl)phenyl)amino) 1,3,5-triazine-2,4-diyl)diimino)bis-,bis-(2-ethyl-hexyl)ester) benzoic acid, 2,4-dihydroxy-benzophenone (Benzophenone-1), 2,2′-dihydroxy-4-methoxy-benzophenone (dioxybenzone, Benzophenone-8), 2,2′-dihydroxy-4,4′-dimethoxy-benzophenone, 2,2′,4,4′-tetrahydroxy-benzophenone, 2-hydroxy-4-methoxy-4′-methyl-benzophenone, hydroxymethoxy-benzophenone sulfonic acid, (2-Hydroxy-4-methoxyphenyl)-phenylmethanone (Benzophenone-3), 2-hydroxy-4-methoxy-benzophenone-5-sulfonic acid (Benzophenone-4) and its salts, 4-phenyl-benzophenone, 2-ethylhexyl-4′-phenyl-benzophenone-2-carboxylate, 2-hydroxy-4-n-octoxy-benzophenone, 4-hydroxy-3-carboxy-benzophenone, 2-hydroxy-4-methoxybenzophenone, 4-(2-β-glucopyranosiloxy)-propoxy-2-hydroxybenzophenone, dihydroxy-dimethoxy-disulfobenzophenone, 2-benzothiazol-2-ylphenol, [2,4′-dihydroxy-3-(2H-benzotriazol-2-yl)-5-(1,1,3,3-tetramethylbutyl)-2′-n-octoxy-5′-benzoyl]-diphenylmethane, 2,2′-hydroxy-5-methylphenyl-benzotriazole, 2-(2′-hydroxy-5′-t-octylphenyl)benzotriazole, 2-(2′-hydroxy-5′-methylphenyl)benzotriazole, 2,2′-methylene-bis-6-(2H-benzotriazol-2-yl)-4-(tetramethyl-butyl)-1,1,3,3-phenol, 2-benzoxazole-2-yl-4-methylphenol, 2-phenyl-5-methylbenzoxazole, 1,4-phenylenebis(2-benzoxazolyl), 1,3-phenylenebis(2-benzoxazolyl), 1,2-phenylenebis(2-benzoxazolyl), 2-(2-benzofuryl)-benzoxazole, 2-(benzofuryl)-5-methylbenzoxazole, 2-(3-methyl-2-benzofuryl)benzoxazole, 3-(4′-methylbenzylidene)-D,L-camphor, terephthalylidene dicamphor sulphonic acid, 3-benzylidene-D,L-camphor, 4-benzylidene-D,L-camphor, di-tert-butyl hydroxybenzylidene camphor, benzylidene camphor sulphonic acid, camphor benzalkonium methosulfate, polyacrylamido-methyl benzylidene camphor, ethylhexyl methoxycinnamate, diethanolamine p-methoxy cinnamate, isopropyl-p-acetamido cinnamate, octyl cinnamate, ethyl-4-isopropylcinnamate, methyl-2,5-diisopropyl cinnamate, ethyl-2,4-diisopropylcinnamate, methyl-2,4-diisopropylcinnamate, propyl-p-methoxycinnamate, isopropyl-p-methoxycinnamate, diisopropylcinnamate esters, isoamyl-p-methoxycinnamate, cyclohexyl-p-methoxycinnamate, ethyl-α-cyano-β-phenylcinnamate, 2-ethylhexyl-α-cyano-β-phenylcinnamate, glycerin-mono-2-ethyl-hexanoyl-diparamethoxycinnamate, glyceryl octanoate-di-p-methoxycinnamate, DEA-methoxycinnamate, methyl-bis(trimethyl-siloxy)silyl-isopentyl-trimethoxycinnamate, ethyl-α-cyano-3,5-dimethoxy-4-hydroxy cinnamate, ethyl-α-acetyl-3,5-dimethoxy-4-hydroxy cinnamate, iso-propyl-α-acetyl-3,5-dimethoxy-4-hydroxy cinnamate, iso-amyl-α-acetyl-3,5-dimethoxy-4-hydroxy cinnamate, 2-ethylhexyl-α-acetyl-3,5-dimethoxy-4-hydroxy cinnamate, ethyl-α-cyano-3-methoxy-4-hydroxy cinnamate, ethyl-α-acetyl-3-methoxy-4-hydroxy cinnamate, iso-propyl-α-acetyl-3-methoxy-4-hydroxy cinnamate, iso-amyl-α-acetyl-3-methoxy-4-hydroxy cinnamate, 2-ethylhexyl-α-acetyl-3-methoxy-4-hydroxy cinnamate, 7-ethylamino-4-methylcoumarin, 7,8-dihydroxycoumarin, 6,7-dihydroxycoumarin, 7-hydroxycoumarin, 4-methyl-7-hydroxycoumarin, diacetycurcumin, tetrahydrocurcumin diacetate, dianisoylmethane, dibenzalazine, butyl methoxydibenzoylmethane (Avobenzone), 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, ferulic acid, dimethicodiethylbenzalmalonate, diethyl-3,5-dimethoxy-4-hydroxy benzylidene malonate, di-(2-ethylhexyl)-3,5-dimethoxy-4-hydroxy benzylidene malonate, diisoamyl-3,5-dimethoxy-4-hydroxy benzylidene malonate, didodecyl-3,5-dimethoxy-4 hydroxy benzylidene malonate, dipalmitoyl-3,5-dimethoxy-4-hydroxy benzylidene malonate, di-isopropyl-3,5-dimethoxy-4-hydroxy benzylidene malonate, diethyl-3-methoxy-4-hydroxy benzylidene malonate, di-(2-ethylhexyl)-3-methoxy-4-hydroxy benzylidene malonate, diisoamyl-3-methoxy-4-hydroxy benzylidene malonate, didodecyl-3-methoxy-4-hydroxy benzylidene malonate, dipalmitoyl-3-methoxy-4-hydroxy benzylidene malonate, di-isopropyl-3-methoxy-4-hydroxy benzylidene malonate, hydrolyzed aspergillus sp. extract ferment, cerium oxide, iron oxide, manganese oxide, titanium dioxide, zinc oxide, zinc cerium oxide, zirconium oxide, 4-nitrobenzamide, 4-nitrobenzoic acid, p-aminobenzoic acid (PABA), PABA-monoglycerinester, N,N-dipropoxy-PABA-ethylester, N,N-diethoxy PABA-ethylester, N,N-dimethyl PABA-ethylester, N,N-dimethyl PABA-butylester, PEG-25 PABA, N,N-dimethyl PABA-amylester, N,N-dimethyl PABA-octlylester (2-ethylhexyl 4-(dimethylamino)benzoate, Padimate O), octyl dimethyl PABA, ethyl-dihydroxypropyl-PABA, isoamyl dimethyl PABA (3-methylbutyl 4-(dimethylamino)benzoate, Padimate A), 1-(3,4-dimethoxyphenyl)-4,4-dimethyl-1,3-pentanedione, 5-(3,3-Dimethyl-2-norbornyliden)-3-pentane-2-one, 1-(4-methoxyphenyl)-3-(4-tert-butylphenyl)propane-1,3-dione, dipropylene glycol salicylate, 2-ethylhexyl 2-cyano-3,3-diphenyl-2-propenoate (Octocrylene), hydrolyzed soy extract, hydrolyzed linseed extract, sunflower seed extract, grape seed extract, pinus pinaster bark extract, pinus pinaster bud extract, ginseng extract ferment, spirulina platensis powder, phenyl salicylate, p-tert-butylphenyl salicylate, p-octylphenyl salicylate, 2-ethylhexyl salicylate (octyl salicylate), amyl salicylate, menthyl salicylate, homomethyl salicylate, benzyl salicylate, p-isopropanol-phenyl salicylate, TEA-salicylate, 2-(2H-benzotriazol-2-yl)-4-methyl-6-[2-methyl-3-[1,3,3,3-tetramethyl-1-[(trimethylsilyl)oxy]disiloxanyl]propyl]-phenol (Mexoryl XL), 2-[p-(tert-butylamido)anilino]-4,6-bis[p-(2′-ethylhexyl-1′-oxycarbonyl)anilino]-1,3,5-triazine (INCI name: Diethylhexyl Butamido Triazone), 2,4,6-tris[4-(2-ethylhexyl-oxycarbonyl)-anilino]-1,3,5-triazine (INCI name: Ethylhexyl Triazone), (1,3,5)-triazine-2,4-bis((4-2-ethyl-hexyloxy)-2-hydroxy)-phenyl)-6-(4-methoxy-phenyl) (bemotrizinol), octyl triazone (CAS-no.: 88122-99-0), 2,4-bis(2,4-dihydroxyphenyl)-6-(1-methyl-1H-pyrazol-5-yl)-1,3,5-triazine, 2,4-bis{[4-(2-ethylhexyloxy)-2-hydroxy]phenyl}-6-(1-methyl-1H-pyrazol-5-yl)-1,3,5-triazine, 2-[2,4-bis(2-ethylhexyloxy)phenyl]-4-[4-(2-ethylhexyloxy)-2-hydroxyphenyl]-6-(1-methyl-1H-pyrazol-5-yl)-1,3,5-triazine, 2,4-bis(2,4-dihydroxyphenyl)-6(1-phenyl-1H-pyrazol-5-yl)-1,3,5-triazine, 2,4-bis{[4-(2-ethylhexyloxy)-2-hydroxy]phenyl}-6-(1-phenyl-1H-pyrazol-5-yl)-1,3,5-triazine, 2,4-bis[4-(butoxycarbonyl)phenylamino]-6-(1-methyl-1H-pyrazol-5-yl)-1,3,5-triazine, 2,4-bis{4-[(2-(ethylhexyloxy)carbonyl]phenylamino}-6-(1-methyl-1H-pyrazol-5-yl)-1,3,5-triazine, 2,4-bis[4-(butoxycarbonyl)phenylamino]-6-(1-phenyl-1H-pyrazol-5-yl)-1,3,5-triazine, 2,4-bis{4-[(2-ethylhexyloxy)carbonyl]phenylamino}-6-(1-phenyl-1H-pyrazol-5-yl)-1,3,5-triazine and 2-[(4-butoxycarbonyl)phenylamino]-4,6-bis(1-methyl-1H-pyrazol-5-yl)-1,3,5-triazine, 2-(1-benzyl-1H-pyrrole-2-yl)-4,6-bis(2,4-dihydroxyphenyl)-1,3,5-triazine, 2-(1-benzyl-1H-pyrrole-2-yl)-4,6-bis[4-(2-ethylhexyloxy)-2-hydroxyphenyl]-1,3,5-triazine, 2-(1-benzyl-1H-pyrrole-2-yl)-4,6-bis[4-(butoxycarbonyl)phenylamino]-1,3,5-triazine, 2-(1-benzyl-1H-pyrrole-2-yl)-4,6-bis(biphenyl-4-ylamino)-1,3,5-triazine, 2-(1-benzyl-1H-pyrrole-2-yl)-4,6-bis(4-benzoylphenylamino)-1,3,5-triazine, 2-(1-benzyl-1H-pyrrole-2-yl)-4,6-bis(9-oxo-9H-fluoren-3-ylamino)-1,3,5-triazine, 2-(1-benzyl-1H-pyrrole-2-yl)-4,6-bis[4-(imidazo[1,2-a]pyridin-2-yl)phenylamino]-1,3,5-triazine, 2-(1-benzhydryl-1H-pyrrole-2-yl)-4,6-bis[4-(butoxycarbonyl)phenylamino]-1,3,5-triazine, 2-(1-benzyl-1H-pyrrole-2-yl)-4,6-bis[4-(6,6-dimethyl-4-oxo-4,5,6,7-tetrahydroindol-1-yl)phenylamino]-1,3,5-triazine, 2-(1-benzyl-1H-pyrrole-2-yl)-4,6-bis[4-((2-ethylhexyloxy)carbonyl)phenylamino]-1,3,5-triazine, 2-(1-benzyl-1H-pyrrole-2-yl)-4,6-bis[4-1H-imidazo[1,2-a]pyridin-2-yl)phenylamino]-1,3,5-triazine, 2-(1-benzyl-1H-pyrrole-2-yl)-4,6-bis[4-(1H-benzo[d]imidazol-2-yl)phenylamino]-1,3,5-triazine, 2-(1-benzyl-1H-pyrrole-2-yl)-4,6-bis[4-(1H-pyrazol-1-yl)phenylamino]-1,3,5-triazine, 2-(1-benzyl-1H-pyrrole-2-yl)-4,6-bis[naphthalen-2-ylamino]-1,3,5-triazine, 2-(1-benzyl-1H-pyrrole-2-yl)-4,6-bis[4-((E)-3-ethoxy-3-oxoprop-1-enyl)phenylamino]-1,3,5-triazine, 2-(1-benzyl-1H-pyrrole-2-yl)-4,6-bis[4-(E)-styrylphenylamino]-1,3,5-triazine, urocanic acid, ethyl-urocanic acid, talc, kaolin, calcium carbonate, bisoctrizole (CAS-no.: 103597-45-I), bornelone (CAS-no.: 2226-11-1), 2-Hydroxy-1,4-naphthochinone (Lawsone, CAS-no.: 83-72-7), petrolatum red, phenol sulphonate, sodium 3,4-dimethylphenyl-glyoxylate, 3,3′-(1,4-phenylenedimethylene-bis-(7,7-dimethyl-2-oxobicyclo-[2,2,1]hept-1-yl-methanesulfonic acid, α-(2-oxoborn-3-ylidene)-toluene-4-sulphonic acid, 2-(2H-benzotriazole-2-yl)-4-methyl-6-[2-methyl-3-[1,3,3,3-tetramethyl-1-[(trimethylsilyl)oxy]disiloxanyl]propyl]-phenol (INCI name: drometrizole trisiloxane, DTS), digalloyl trioleate, dimethicone/PEG-15 crosspolymer, ethylhexyl-bis-isopentyl-benzoxazolyl-phenyl melamine and 2-ethylhexyl-dimethoxy-benzylidene-dioxo-imidazolidine propionate;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate of the afore mentioned compounds.

21. A combination according to at least one of the preceding claims, characterized in that the UV-absorbing active substance (A) is at least one heptaazaphenalene compound according to claim 13,
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof;
and the UV-absorbing active substance (B)
is at least one inorganic compound selected from the group comprising cerium oxide, iron oxide, manganese oxide, titanium dioxide, zinc oxide, zinc cerium oxide and zirconium oxide;
and/or
is at least one UV-absorbing organic compound selected from the group consisting of 2-ethylhexyl 2-cyano-3,3′-diphenylacrylate, ethyl 2-cyano-3,3′-diphenylacrylate, polymer of N-{(2 and 4)[(2-oxoborn-3-ylidene)methyl]benzyl}acrylamide, methyl 4-aminobenzoate, butyl 4-aminobenzoate, 5-methyl-2-(1-methylethyl)-cyclohexyl 2-aminobenzoate, glyceryl aminobenzoate, 2-ethylhexyl-p-dimethylethyl-aminobenzoate, 4-aminobenzamide, homomethyl-N-acetyl-anthranilic acid, menthyl antrhanilate, 2-(1H-benzimidazol-2-yl)-4-methoxyphenol, 2,2′-bis(benzimidazole), monosodium salt of 2-2′-bis-(1,4-phenylene)1H-benzimidazole-4,6-disulphonic acid, 2-phenylbenzimidazole-5-sulphonic acid, 5,5′,6,6′-tetramethyl-2,2′-bis(benzimidazole), 5,5′-dimethyl-2,2′-bis(benzimidazole), 6-methoxy-2,2′-bis(benzimidazole), 1,4-phenylenebis(2-benzimidazolyl), 1,2-phenylenebis)benzimidazolyl), 1,4-phenylenebis(N-2-ethylhexyl-2-benzimidazolyl), 1,4-phenylenebis(N-tri-methylsilylmethyl-2-benzimidazolyl), 2-(1H-benzimidazol-2-yl)benzothiazole, 2-(1H-benzimidazol-2-yl)benzoxazole, N,N′-dimethyl-2,2′-bis(benzimidazole), phenylbenzimidazole-5-sulfonic acid (Ensulizole) and its salts, 2-2′-bis-(1,4-phenylene)1H-benzimidazole-4,6-disulphonic acid, 3-methylbutyl 4-(dimethylamino)benzoate, 3,3,5-trimethylcyclohexyl 2-hydroxybenzoate (Homosalate), ethyl 4-aminobenzoate, 4,4-((6-(((1,1-dimethylethyl)amino)carbonyl)phenyl)amino) 1,3,5-triazine-2,4-diyl)diimino)bis-,bis-(2-ethyl-hexyl)ester) benzoic acid, 2,4-dihydroxy-benzophenone (Benzophenone-1), 2,2′-dihydroxy-4-methoxy-benzophenone (dioxybenzone, Benzophenone-8), 2,2′-dihydroxy-4,4′-dimethoxy-benzophenone, 2,2′,4,4′-tetrahydroxy-benzophenone, 2-hydroxy-4-methoxy-4′-methyl-benzophenone, hydroxymethoxy-benzophenone sulfonic acid, (2-Hydroxy-4-methoxyphenyl)-phenylmethanone (Benzophenone-3), 2-hydroxy-4-methoxy-benzophenone-5-sulfonic acid (Benzophenone-4) and its salts, 4-phenyl-benzophenone, 2-ethylhexyl-4′-phenyl-benzophenone-2-carboxylate, 2-hydroxy-4-n-octoxy-benzophenone, 4-hydroxy-3-carboxy-benzophenone, 2-hydroxy-4-methoxybenzophenone, 4-(2-β-glucopyranosiloxy)-propoxy-2-hydroxybenzophenone, dihydroxy-dimethoxy-disulfobenzophenone, 2-benzothiazol-2-ylphenol, [2,4′-dihydroxy-3-(2H-benzotriazol-2-yl)-5-(1,1,3,3-tetramethylbutyl)-2′-n-octoxy-5′-benzoyl]-diphenylmethane, 2,2′-hydroxy-5-methylphenyl-benzotriazole, 2-(2′-hydroxy-5′-t-octylphenyl)benzotriazole, 2-(2′-hydroxy-5′-methylphenyl)benzotriazole, 2,2′-methylene-bis-6-(2H-benzotriazol-2-yl)-4-(tetramethyl-butyl)-1,1,3,3-phenol, 2-benzoxazole-2-yl-4-methylphenol, 2-phenyl-5-methylbenzoxazole, 1,4-phenylenebis(2-benzoxazolyl), 1,3-phenylenebis(2-benzoxazolyl), 1,2-phenylenebis(2-benzoxazolyl), 2-(2-benzofuryl)-benzoxazole, 2-(benzofuryl)-5-methylbenzoxazole, 2-(3-methyl-2-benzofuryl)benzoxazole, 3-(4′-methylbenzylidene)-D, L-camphor, terephthalylidene dicamphor sulphonic acid, 3-benzylidene-D,L-camphor, 4-benzylidene-D,L-camphor, di-tert-butyl hydroxybenzylidene camphor, benzylidene camphor sulphonic acid, camphor benzalkonium methosulfate, polyacrylamido-methyl benzylidene camphor, ethylhexyl methoxycinnamate, diethanolamine p-methoxy cinnamate, isopropyl-p-acetamido cinnamate, octyl cinnamate, ethyl-4-isopropylcinnamate, methyl-2,5-diisopropyl cinnamate, ethyl-2,4-diisopropylcinnamate, methyl-2,4-diisopropylcinnamate, propyl-p-methoxycinnamate, isopropyl-p-methoxycinnamate, diisopropylcinnamate esters, isoamyl-p-methoxycinnamate, cyclohexyl-p-methoxycinnamate, ethyl-α-cyano-β-phenylcinnamate, 2-ethylhexyl-α-cyano-β-phenylcinnamate, glycerin-mono-2-ethyl-hexanoyl-diparamethoxycinnamate, glyceryl octanoate-di-p-methoxycinnamate, DEA-methoxycinnamate, methyl-bis(trimethyl-siloxy)silyl-isopentyl-trimethoxycinnamate, ethyl-α-cyano-3,5-dimethoxy-4-hydroxy cinnamate, ethyl-α-acetyl-3,5-dimethoxy-4-hydroxy cinnamate, iso-propyl-α-acetyl-3,5-dimethoxy-4-hydroxy cinnamate, iso-amyl-α-acetyl-3,5-dimethoxy-4-hydroxy cinnamate, 2-ethylhexyl-α-acetyl-3,5-dimethoxy-4-hydroxy cinnamate, ethyl-α-cyano-3-methoxy-4-hydroxy cinnamate, ethyl-α-acetyl-3-methoxy-4-hydroxy cinnamate, iso-propyl-α-acetyl-3-methoxy-4-hydroxy cinnamate, iso-amyl-α-acetyl-3-methoxy-4-hydroxy cinnamate, 2-ethylhexyl-α-acetyl-3-methoxy-4-hydroxy cinnamate, 7-ethylamino-4-methylcoumarin, 7,8-dihydroxycoumarin, 6,7-dihydroxycoumarin, 7-hydroxycoumarin, 4-methyl-7-hydroxycoumarin, diacetycurcumin, tetrahydrocurcumin diacetate, dianisoylmethane, dibenzalazine, butyl methoxydibenzoylmethane (Avobenzone), 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, ferulic acid, dimethicodiethylbenzalmalonate, diethyl-3,5-dimethoxy-4-hydroxy benzylidene malonate, di-(2-ethylhexyl)-3,5-dimethoxy-4-hydroxy benzylidene malonate, diisoamyl-3,5-dimethoxy-4-hydroxy benzylidene malonate, didodecyl-3,5-dimethoxy-4 hydroxy benzylidene malonate, dipalmitoyl-3,5-dimethoxy-4-hydroxy benzylidene malonate, di-isopropyl-3,5-dimethoxy-4-hydroxy benzylidene malonate, diethyl-3-methoxy-4-hydroxy benzylidene malonate, di-(2-ethylhexyl)-3-methoxy-4-hydroxy benzylidene malonate, diisoamyl-3-methoxy-4-hydroxy benzylidene malonate, didodecyl-3-methoxy-4-hydroxy benzylidene malonate, dipalmitoyl-3-methoxy-4-hydroxy benzylidene malonate, di-isopropyl-3-methoxy-4-hydroxy benzylidene malonate, hydrolyzed aspergillus sp. extract ferment, 4-nitrobenzamide, 4-nitrobenzoic acid, p-aminobenzoic acid (PABA), PABA-monoglycerinester, N,N-dipropoxy-PABA-ethylester, N,N-diethoxy PABA-ethylester, N,N-dimethyl PABA-ethylester, N,N-dimethyl PABA-butylester, PEG-25 PABA, N,N-dimethyl PABA-amylester, N,N-dimethyl PABA-octlylester (2-ethylhexyl 4-(dimethylamino)benzoate, Padimate O), octyl dimethyl PABA, ethyl-dihydroxypropyl-PABA, isoamyl dimethyl PABA (3-methylbutyl 4-(dimethylamino)benzoate, Padimate A), 1-(3,4-dimethoxyphenyl)-4,4-dimethyl-1,3-pentanedione, 5-(3,3-Dimethyl-2-norbornyliden)-3-pentane-2-one, 1-(4-methoxyphenyl)-3-(4-tert-butylphenyl)propane-1,3-dione, dipropylene glycol salicylate, 2-ethylhexyl 2-cyano-3,3-diphenyl-2-propenoate (Octocrylene), hydrolyzed soy extract, hydrolyzed linseed extract, sunflower seed extract, grape seed extract, pinus pinaster bark extract, pinus pinaster bud extract, ginseng extract ferment, spirulina platensis powder, phenyl salicylate, p-tert-butylphenyl salicylate, p-octylphenyl salicylate, 2-ethylhexyl salicylate (octyl salicylate), amyl salicylate, menthyl salicylate, homomethyl salicylate, benzyl salicylate, p-isopropanol-phenyl salicylate, TEA-salicylate, 2-(2H-benzotriazol-2-yl)-4-methyl-6-[2-methyl-3-[1,3,3,3-tetramethyl-1-[(trimethylsilyl)oxy]disiloxanyl]propyl]-phenol (Mexoryl XL), 2-[p-(tert-butylamido)anilino]-4,6-bis[p-(2′-ethylhexyl-1′-oxycarbonyl)anilino]-1,3,5-triazine (INCI name: Diethylhexyl Butamido Triazone), 2,4,6-tris[4-(2-ethylhexyl-oxycarbonyl)-anilino]-1,3,5-triazine (INCI name: Ethylhexyl Triazone), (1,3,5)-triazine-2,4-bis((4-2-ethyl-hexyloxy)-2-hydroxy)-phenyl)-6-(4-methoxy-phenyl) (bemotrizinol), octyl triazone (CAS-no.: 88122-99-0), 2,4-bis(2,4-dihydroxyphenyl)-6-(1-methyl-1H-pyrazol-5-yl)-1,3,5-triazine, 2,4-bis{[4-(2-ethylhexyloxy)-2-hydroxy]phenyl}-6-(1-methyl-1H-pyrazol-5-yl)-1,3,5-triazine, 2-[2,4-bis(2-ethylhexyloxy)phenyl]-4-[4-(2-ethylhexyloxy)-2-hydroxyphenyl]-6-(1-methyl-1H-pyrazol-5-yl)-1,3,5-triazine, 2,4-bis(2,4-dihydroxyphenyl)-6(1-phenyl-1H-pyrazol-5-yl)-1,3,5-triazine, 2,4-bis{[4-(2-ethylhexyloxy)-2-hydroxy]phenyl}-6-(1-phenyl-1H-pyrazol-5-yl)-1,3,5-triazine, 2,4-bis[4-(butoxycarbonyl)phenylamino]-6-(1-methyl-1H-pyrazol-5-yl)-1,3,5-triazine, 2,4-bis{4-[(2-(ethylhexyloxy)carbonyl]phenylamino}-6-(1-methyl-1H-pyrazol-5-yl)-1,3,5-triazine, 2,4-bis[4-(butoxycarbonyl)phenylamino]-6-(1-phenyl-1H-pyrazol-5-yl)-1,3,5-triazine, 2,4-bis{4-[(2-ethylhexyloxy)carbonyl]phenylamino}-6-(1-phenyl-1H-pyrazol-5-yl)-1,3,5-triazine and 2-[(4-butoxycarbonyl)phenylamino]-4,6-bis(1-methyl-1H-pyrazol-5-yl)-1,3,5-triazine, 2-(1-benzyl-1H-pyrrole-2-yl)-4,6-bis(2,4-dihydroxyphenyl)-1,3,5-triazine, 2-(1-benzyl-1H-pyrrole-2-yl)-4,6-bis[4-(2-ethylhexyloxy)-2-hydroxyphenyl]-1,3,5-triazine, 2-(1-benzyl-1H-pyrrole-2-yl)-4,6-bis[4-(butoxycarbonyl)phenylamino]-1,3,5-triazine, 2-(1-benzyl-1H-pyrrole-2-yl)-4,6-bis(biphenyl-4-ylamino)-1,3,5-triazine, 2-(1-benzyl-1H-pyrrole-2-yl)-4,6-bis(4-benzoylphenylamino)-1,3,5-triazine, 2-(1-benzyl-1H-pyrrole-2-yl)-4,6-bis(9-oxo-9H-fluoren-3-ylamino)-1,3,5-triazine, 2-(1-benzyl-1H-pyrrole-2-yl)-4,6-bis[4-(imidazo[1,2-a]pyridin-2-yl)phenylamino]-1,3,5-triazine, 2-(1-benzhydryl-1H-pyrrole-2-yl)-4,6-bis[4-(butoxycarbonyl)phenylamino]-1,3,5-triazine, 2-(1-benzyl-1H-pyrrole-2-yl)-4,6-bis[4-(6,6-dimethyl-4-oxo-4,5,6,7-tetrahydroindol-1-yl)phenylamino]-1,3,5-triazine, 2-(1-benzyl-1H-pyrrole-2-yl)-4,6-bis[4-((2-ethylhexyloxy)carbonyl)phenylamino]-1,3,5-triazine, 2-(1-benzyl-1H-pyrrole-2-yl)-4,6-bis[4-1H-imidazo[1,2-a]pyridin-2-yl)phenylamino]-1,3,5-triazine, 2-(1-benzyl-1H-pyrrole-2-yl)-4,6-bis[4-(1H-benzo[d]imidazol-2-yl)phenylamino]-1,3,5-triazine, 2-(1-benzyl-1H-pyrrole-2-yl)-4,6-bis[4-(1H-pyrazol-1-yl)phenylamino]-1,3,5-triazine, 2-(1-benzyl-1H-pyrrole-2-yl)-4,6-bis[naphthalen-2-ylamino]-1,3,5-triazine, 2-(1-benzyl-1H-pyrrole-2-yl)-4,6-bis[4-((E)-3-ethoxy-3-oxoprop-1-enyl)phenylamino]-1,3,5-triazine, 2-(1-benzyl-1H-pyrrole-2-yl)-4,6-bis[4-(E)-styrylphenylamino]-1,3,5-triazine, urocanic acid, ethyl-urocanic acid, talc, kaolin, calcium carbonate, bisoctrizole (CAS-no.: 103597-45-I), bornelone (CAS-no.: 2226-11-1), 2-Hydroxy-1,4-naphthochinone (Lawsone, CAS-no.: 83-72-7), petrolatum red, phenol sulphonate, sodium 3,4-dimethylphenyl-glyoxylate, 3,3′-(1,4-phenylenedimethylene-bis-(7,7-dimethyl-2-oxobicyclo-[2,2,1]hept-1-yl-methanesulfonic acid, α-(2-oxoborn-3-ylidene)-toluene-4-sulphonic acid, 2-(2H-benzotriazole-2-yl)-4-methyl-6-[2-methyl-3-[1,3,3,3-tetramethyl-1-[(trimethylsilyl)oxy]disiloxanyl]propyl]-phenol (INCI name: drometrizole trisiloxane, DTS), digalloyl trioleate, dimethicone/PEG-15 crosspolymer, ethylhexyl-bis-isopentyl-benzoxazolyl-phenyl melamine and 2-ethylhexyl-dimethoxy-benzylidene-dioxo-imidazolidine propionate;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof.

22. A combination according to at least one of the preceding claims, characterized in that the UV-absorbing active substance (A) is at least one heptaazaphenalene compound according to claim 13;
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof;
and the UV-absorbing active substance (B)
is at least one inorganic compound selected from the group comprising cerium oxide, iron oxide, manganese oxide, titanium dioxide, zinc oxide, zinc cerium oxide and zirconium oxide;
and/or
is at least one UV-absorbing organic compound selected from the group comprising
2,4-bis(2,4-dihydroxyphenyl)-6-(1-methyl-1H-pyrazol-5-yl)-1,3,5-triazine, 2,4-bis{[4-(2-ethylhexyloxy)-2-hydroxy]phenyl}-6-(1-methyl-1H-pyrazol-5-yl)-1,3,5-triazine, 2-[2,4-bis(2-ethylhexyloxy)phenyl]-4-[4-(2-ethylhexyloxy)-2-hydroxyphenyl]-6-(1-methyl-1H-pyrazol-5-yl)-1,3,5-triazine, 2,4-bis(2,4-dihydroxyphenyl)-6(1-phenyl-1H-pyrazol-5-yl)-1,3,5-triazine, 2,4-bis{[4-(2-ethylhexyloxy)-2-hydroxy]phenyl}-6-(1-phenyl-1H-pyrazol-5-yl)-1,3,5-triazine, 2,4-bis[4-(butoxycarbonyl)phenylamino]-6-(1-methyl-1H-pyrazol-5-yl)-1,3,5-triazine, 2,4-bis{4-[(2-(ethylhexyloxy)carbonyl]phenylamino}-6-(1-methyl-1H-pyrazol-5-yl)-1,3,5-triazine, 2,4-bis[4-(butoxycarbonyl)phenylamino]-6-(1-phenyl-1H-pyrazol-5-yl)-1,3,5-triazine, 2,4-bis{4-[(2-ethylhexyloxy)carbonyl]phenylamino}-6-(1-phenyl-1H-pyrazol-5-yl)-1,3,5-triazine and 2-[(4-butoxycarbonyl)phenylamino]-4,6-bis(1-methyl-1H-pyrazol-5-yl)-1,3,5-triazine, 2-(1-benzyl-1H-pyrrole-2-yl)-4,6-bis(2,4-dihydroxyphenyl)-1,3,5-triazine, 2-(1-benzyl-1H-pyrrole-2-yl)-4,6-bis[4-(2-ethylhexyloxy)-2-hydroxyphenyl]-1,3,5-triazine, 2-(1-benzyl-1H-pyrrole-2-yl)-4,6-bis[4-(butoxycarbonyl)phenylamino]-1,3,5-triazine, 2-(1-benzyl-1H-pyrrole-2-yl)-4,6-bis(biphenyl-4-ylamino)-1,3,5-triazine, 2-(1-benzyl-1H-pyrrole-2-yl)-4,6-bis(4-benzoylphenylamino)-1,3,5-triazine, 2-(1-benzyl-1H-pyrrole-2-yl)-4,6-bis(9-oxo-9H-fluoren-3-ylamino)-1,3,5-triazine, 2-(1-benzyl-1H-pyrrole-2-yl)-4,6-bis[4-(imidazo[1,2-a]pyridin-2-yl)phenylamino]-1,3,5-triazine, 2-(1-benzhydryl-1H-pyrrole-2-yl)-4,6-bis[4-(butoxycarbonyl)phenylamino]-1,3,5-triazine, 2-(1-benzyl-1H-pyrrole-2-yl)-4,6-bis[4-(6,6-dimethyl-4-oxo-4,5,6,7-tetrahydroindol-1-yl)phenylamino]-1,3,5-triazine, 2-(1-benzyl-1H-pyrrole-2-yl)-4,6-bis[4-((2-ethylhexyloxy)carbonyl)phenylamino]-1,3,5-triazine, 2-(1-benzyl-1H-pyrrole-2-yl)-4,6-bis[4-1H-imidazo[1,2-a]pyridin-2-yl)phenylamino]-1,3,5-triazine, 2-(1-benzyl-1H-pyrrole-2-yl)-4,6-bis[4-(1H-benzo[d]imidazol-2-yl)phenylamino]-1,3,5-triazine, 2-(1-benzyl-1H-pyrrole-2-yl)-4,6-bis[4-(1H-pyrazol-1-yl)phenylamino]-1,3,5-triazine, 2-(1-benzyl-1H-pyrrole-2-yl)-4,6-bis[naphthalen-2-ylamino]-1,3,5-triazine, 2-(1-benzyl-1H-pyrrole-2-yl)-4,6-bis[4-((E)-3-ethoxy-3-oxoprop-1-enyl)phenylamino]-1,3,5-triazine, 2-(1-benzyl-1H-pyrrole-2-yl)-4,6-bis[4-(E)-styrylphenylamino]-1,3,5-triazine, 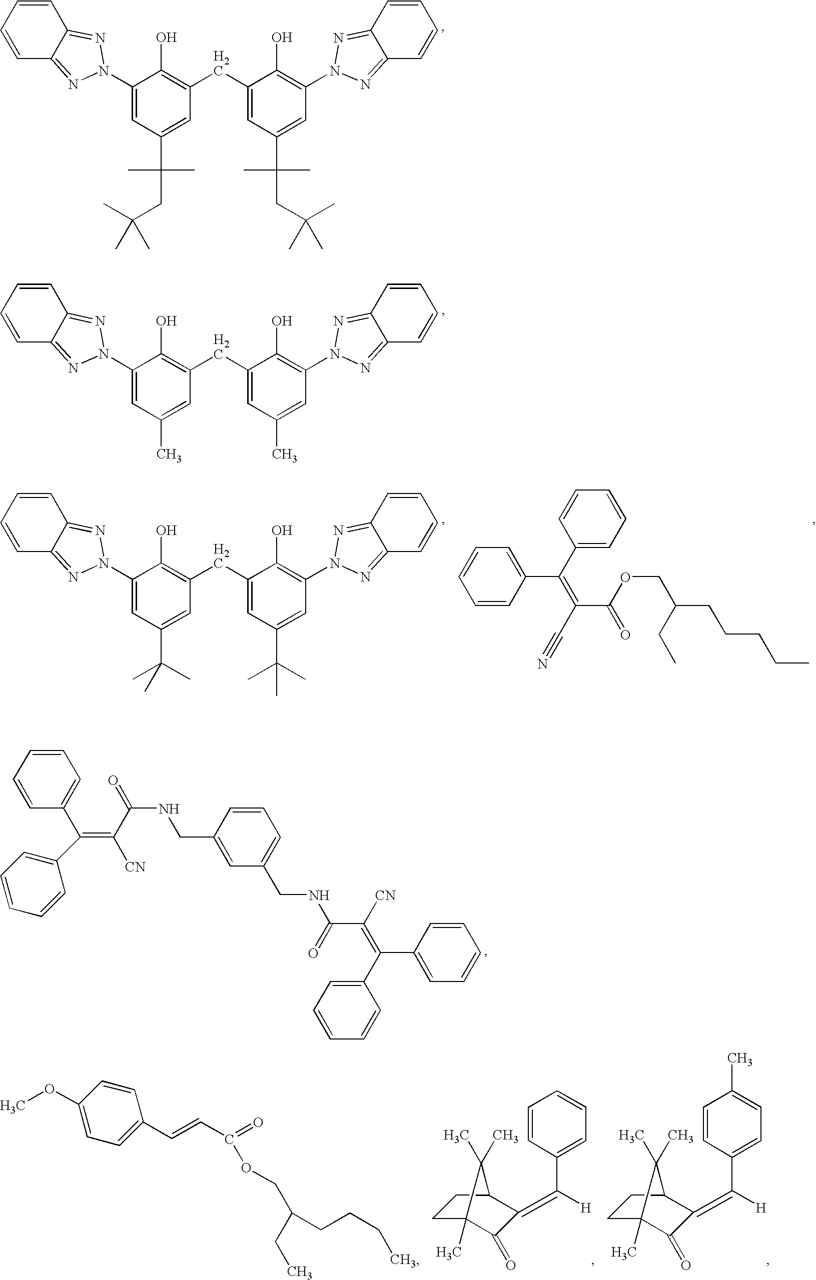 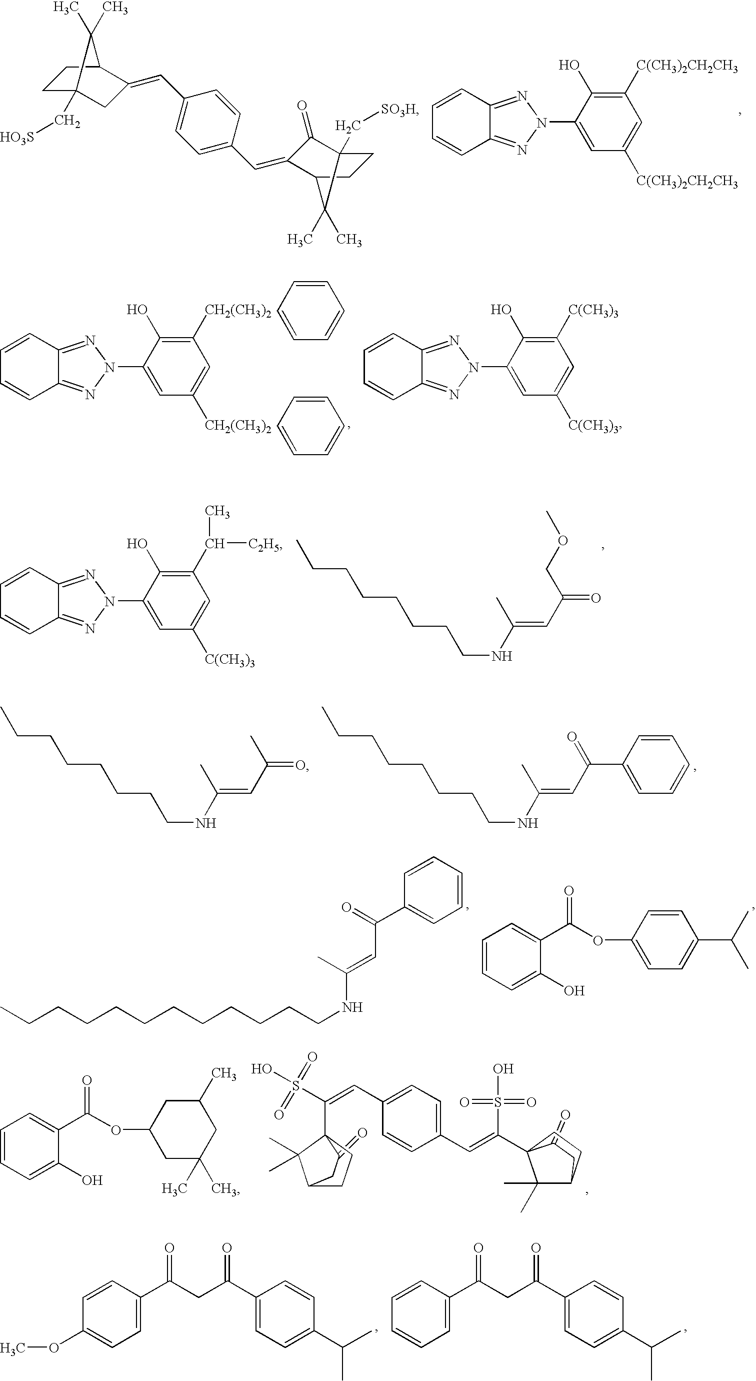 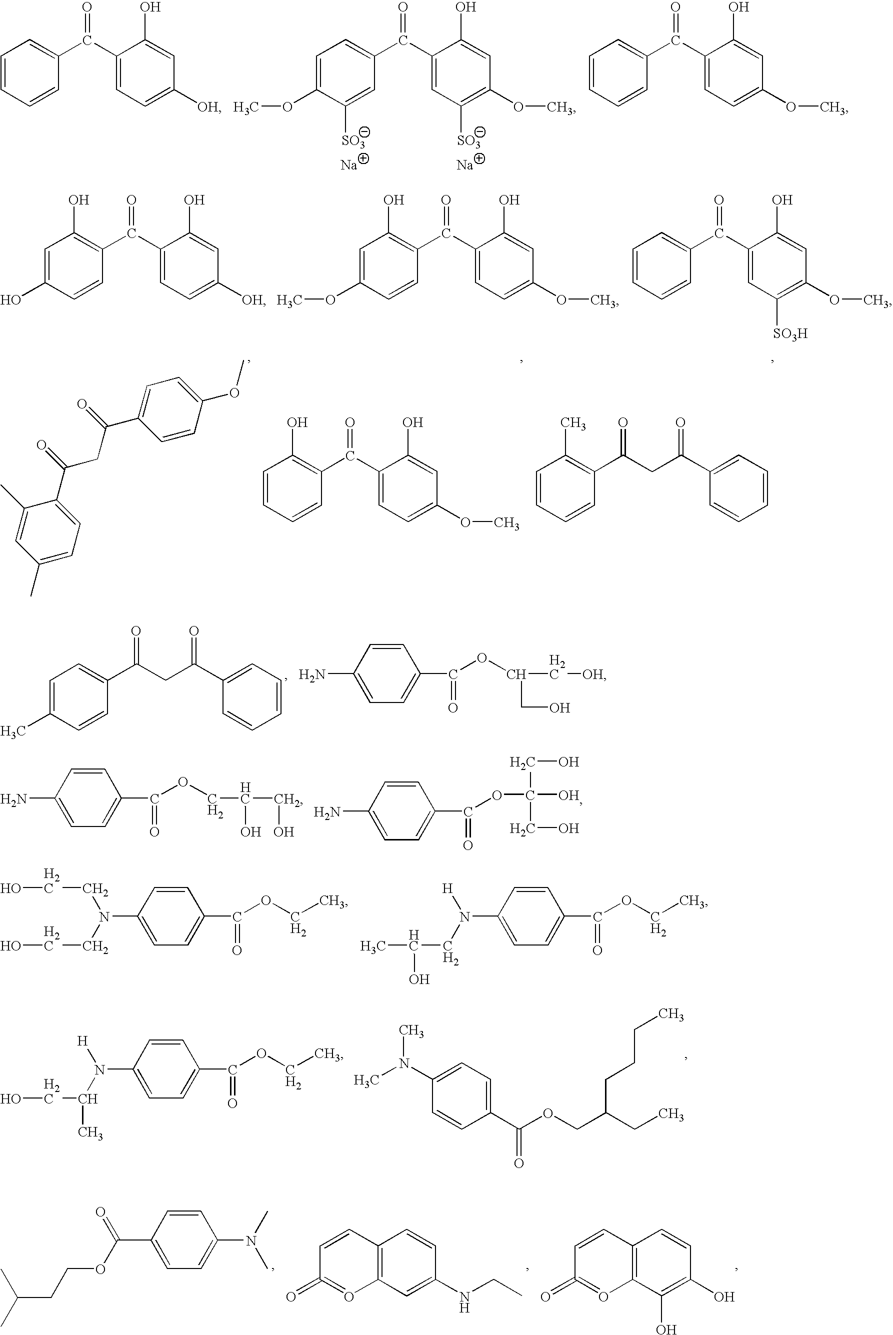 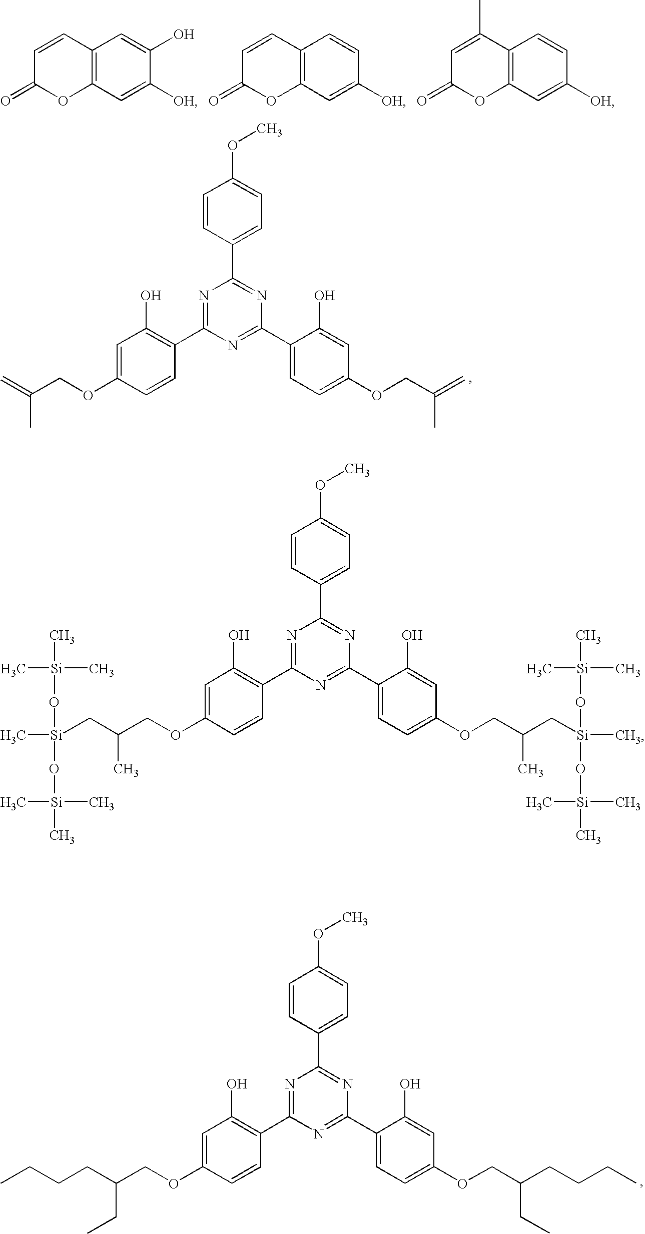 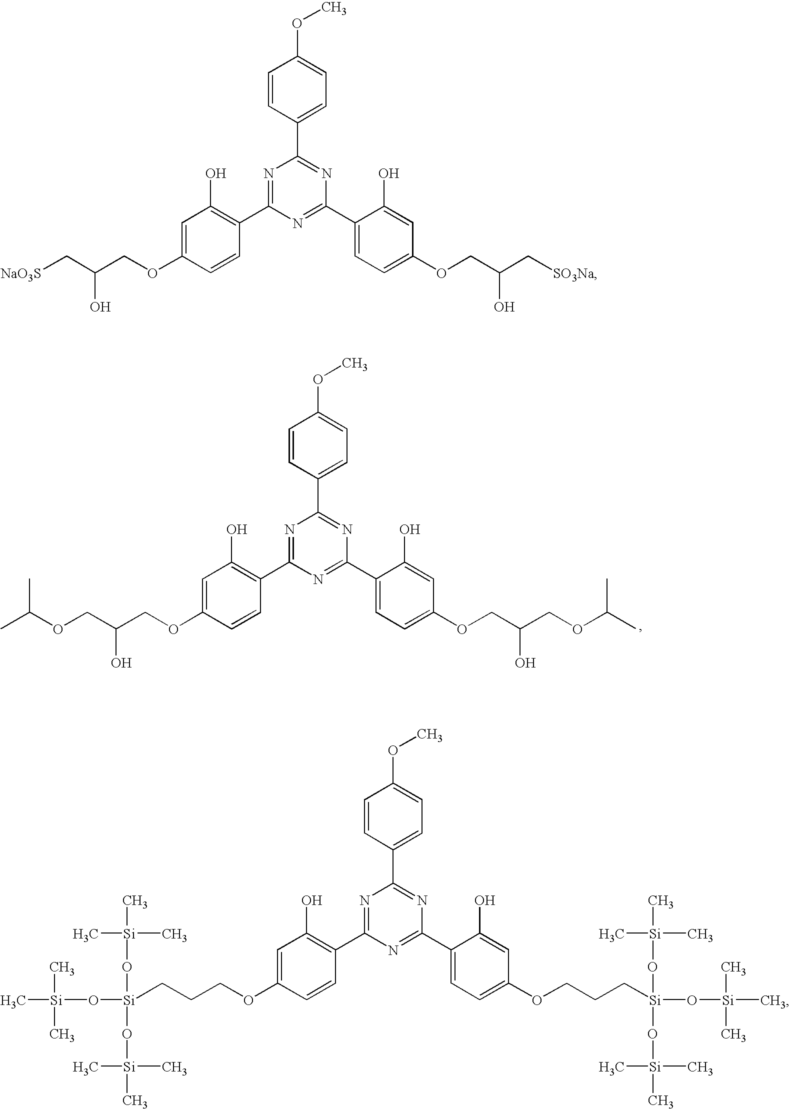 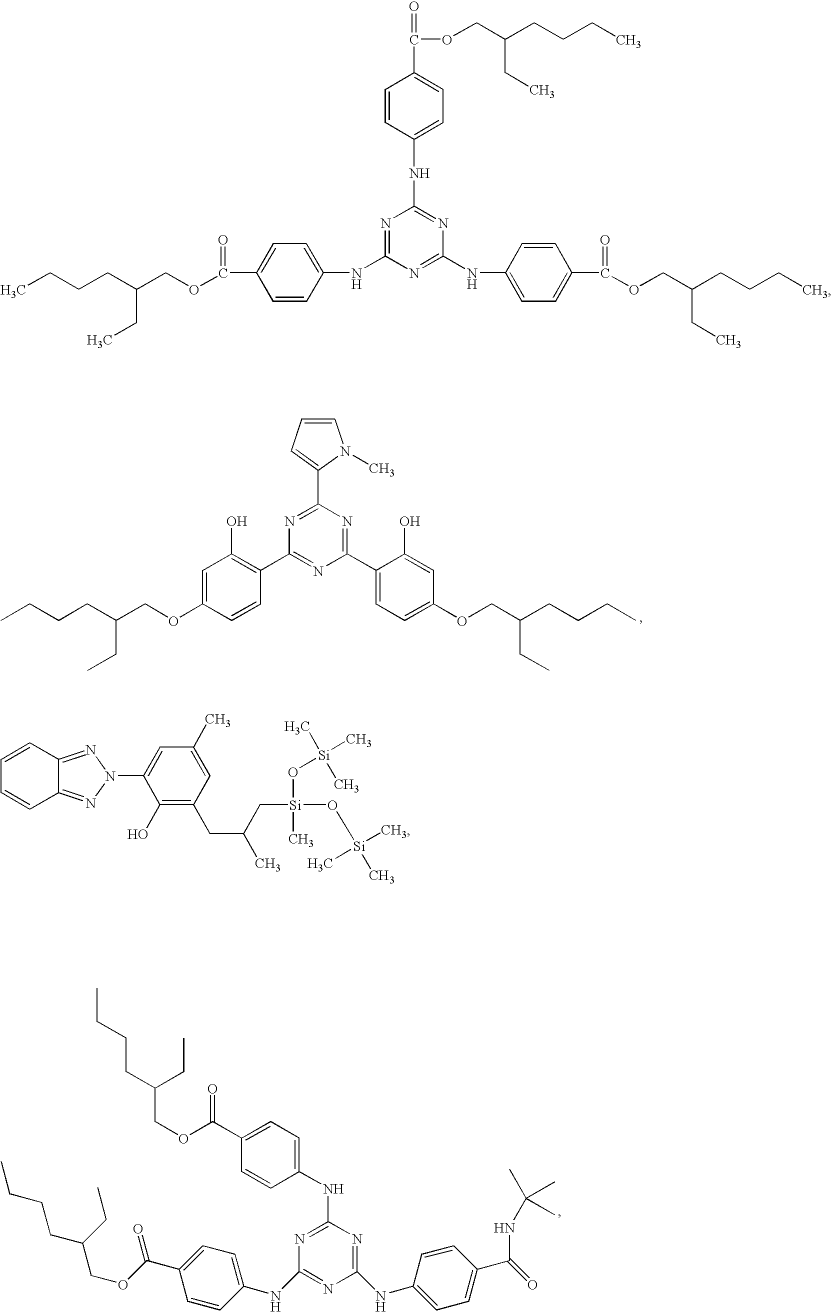 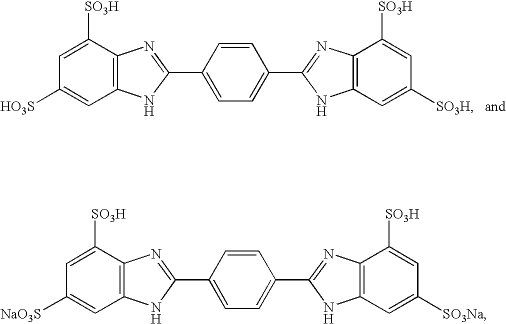
and/or at least one of its stereoisomers, preferably enantiomers or diastereomers, a racemate or a mixture of at least two of its stereoisomers, preferably enantiomers and/or diastereomers in any mixing ratio, or at least one physiologically acceptable salt and/or at least one corresponding solvate thereof.

23. A combination according to at least one of the preceding claims 1 to 22, characterized in that it has an UV-A/UV-B protection ratio in the range from 0.1 to 1, preferably in the range from 0.2 to 1.

24. A combination according to at least one of the preceding claims 1 to 23, characterized in that it absorbs at least 50% of the total UV radiation in a wavelength range from 290 to 400 nm.

25. A combination according to at least one of the preceding claims 1 to 23, characterized in that it absorbs at least 20% of the UV radiation in a wavelength range from 290 to 320 nm and/or at least 20% of the UV radiation in a wavelength range from 320 to 400 nm.

26. A pharmaceutical composition comprising a combination according to at least one of claims 1 to 25 and optionally at least one pharmacologically acceptable auxiliary agent.

27. A cosmetical composition comprising a combination according to at least one of claims 1 to 25, and optionally at least one cosmetical vehicle.

28. A pharmaceutical or cosmetical composition according to claim 26 or 27, characterized in that the amount of the UV-absorbing active substance (A) and/or the UV-absorbing active substance (B) is in each case from 0.01% to 30% per weight, preferably 0.01% to 20% per weight, with respect to the total weight of the composition.

29. A pharmaceutical or cosmetical composition according to any one of claims 26 to 27, characterized in that at least one UV-absorbing active substance (A) or (B) is in micronized form.

30. A pharmaceutical or cosmetical composition according to claim 29, characterized in that the average particle size of the micronized form is in the range of 0.005 to 2 μm.

31. A pharmaceutical or cosmetical composition according to anyone of claims 26 to 30, characterized in that the composition is suitable for a topical application.

32. A pharmaceutical or cosmetical composition according to anyone of claims 26 to 31, characterized in that the composition is formulated in liquid or in semi-solid form.

33. A pharmaceutical or cosmetical composition according to anyone of claims 26 to 32, characterized in that the composition is formulated as liquid, fluid, foam, cream, gel, paste, balsam, spray, ointment, lotion, conditioner, tonic, milk, mousse, emulsion, serum, oil, stick, shampoo, jelly, suspension, dispersion, lacquer, paint, elixir, drop or aerosol.

34. A pharmaceutical or cosmetical composition according to anyone of claims 26 to 33, characterized in that the composition is adapted for an at least once a day application.

35. A cosmetic composition according to anyone of claims 27 to 34, characterized in that the composition is suitable for keeping scalp, skin, hair and/or nails in good conditions.

36. Use of a combination according to anyone of claims 1 to 25 for the preparation of a pharmaceutical composition for the prophylaxis and/or treatment of diseases caused by and/or associated with exposure to ultraviolet radiation, preferably of the lips, the face and/or of the body of mammals, preferably human beings.

37. Use of a combination according to anyone of claims 1 to 25 for the preparation of a pharmaceutical composition for the prophylaxis and/or care and/or treatment against non-desired skin conditions comprising polymorphous light eruption, actinic keratosis, solar urticaria, xeroderma pigmentosum, photoageing and/or chronic actinic dermatitis.

38. Use of a cosmetical combination according to anyone of claims 27 to 35 for the prophylaxis and/or care against non-desired skin conditions comprising polymorphous light eruption, actinic keratosis, solar urticaria, xeroderma pigmentosum, photoageing and/or chronic actinic dermatitis.

39. Use of a cosmetical combination according to anyone of claims 27 to 35 for protecting the skin preferably of the lips, the face and/or of the body of a mammal, preferably a human, against ultraviolet radiation, preferably UV-A and/or UV-B radiation.
